(19) United States
(12) Patent Application Publication
(10) Pub. No.: US 2004/0067903 A1
(43) Pub. Date: Apr. 8, 2004

(54) Nucleic acids encoding a novel regulator of g protein signaling, rgs18, and uses thereof

(76) Inventors: Alison Williams-Gagnon, Wayne, PA (US); David Lee Murray, Westerly, RI (US)

Correspondence Address: Aventis Pharmaceuticals Inc, Patent Department, Route #202 206, PO Box 6800, Bridgewater, NJ, 08807-0800, US

(21) Appl. No.: 10/258,371

(22) PCT Filed: Apr. 26, 2001
(86) PCT No.: PCT/US01/13540

(30) Foreign Application Priority Data
Aug. 2, 2000 (GB) 0018833.4

(51) Int. Cl. C07H 21/04; C07K 14/705; C12P 21/02; C12N 5/06

(52) U.S. Cl. 514/44; 435/69.1; 435/320.1; 435/325; 530/350; 536/23.5

(57) ABSTRACT

The present invention relates to nucleic acids that encode novel Regulator of G protein Signaling RGS18 polypeptides. RGS18 polypeptides are abundantly expressed in platelets and comprise a novel RGS domain (RGS18 domain). The present invention also relates to RGS18 polypeptides. The invention also relates to means for the detection of RGS18 nucleic acids and RGS18 polypeptides. The invention also relates to methods for the detection of activators or inhibitors of RGS18 polypeptides. Finally, the present invention relates to methods of prevention and/or treatment of disorders or conditions associated with platelet activation dysfunction.

### FIELD OF THE INVENTION

The present invention relates to nucleic acids encoding a novel Regulator of G protein Signaling (RGS) protein, RGS18. RGS18 is abundantly expressed in platelets and comprises a novel RGS domain (RGS18 domain). The present invention also relates to nucleic acids encoding a polypeptide comprising the novel RGS18 domain. The present invention also relates to a cDNA encoding the novel full length RGS18 protein. In addition, the present invention relates to the RGS18 protein and polypeptides comprising the novel RGS18 domain. The invention also relates to a recombinant vector comprising a nucleic acid according to the invention. The invention also relates to means for the detection of RGS18 nucleic acids, protein, and RGS18 domain comprising polypeptides. The invention also relates to methods for the detection of activators or inhibitors of RGS18 protein and RGS18 domain comprising polypeptides. Finally, the present invention relates to methods of prevention and/or treatment of disorders or conditions associated with platelet activation dysfunction.

### BACKGROUND OF THE INVENTION

The evolution of multicellular organisms has been dependent on the capacity of cells to communicate with each other and with their immediate environment. Membrane bound receptors have been found to play a crucial role in such communication. They can recognize intercellular messenger molecule such as hormones, neurotransmitters, growth, and development factors as well as sensory messengers, such as odorant, gustative and light. These receptors belong to about five protein families; the most common family is the G protein-coupled receptor family (GPCR). GPCRs are involved in the recognition of messages as diverse as light, odorant, calcium, small molecules including amino acid residues, nucleotides and peptides. Following binding of such messages to GPCRs, signal transduction proceeds via recruitment of G-proteins, activated by binding and hydrolyzing GTP on the intracellular level. Biological actions of GPCR include controlling the activity of intracellular enzymes, ion channels and transport of vesicles via the catalysis of the GDP-GTP exchange on heterotrimeric G proteins (Gα-βγ) (1-4).

A main feature in the structure of GPCR is a central core domain that encompasses seven transmembrane helices (TMI-VI), spaced by three intracellular loops (IPI-III) and three extracellular loops (ECI-III). These three core domains differ in sequence and function among members of the GPCR superfamily, in their N-terminal extracellular domain, C-terminal intracellular domain and their intracellular loops.

Several platelet agonists act via activation of cell surface GPCR initiating intracellular signaling cascades that culminate in platelet aggregation. Signaling through G protein coupled receptors, such as thrombin, thromboxane A₂ and adenosine diphosphate (ADP) is in part responsible for platelet activation events, such as fibrinogen receptor exposure, granule secretion and aggregation (5). Multiple intracellular signaling pathways have been implicated in platelet activation events, although the exact sequence of events and host of intracellular signaling molecules remains undefined.

Regulators of G protein Signaling (RGS) represent a family of proteins that function to dampen signals generated upon stimulation of cell-surface G protein-coupled receptors. First identified in genetic screens of yeast (6, 7) and the nematode Caenorhabditis elegans (8), RGSs were first discovered based on their ability to modulate behavioral responses. Mammalian homologues of these lower eukaryotic RGSs were quickly identified by several methods including yeast two-hybrid (9), homology cloning (10), database searching (8), or subtractive cloning, or expression methods (11-13). The hallmark of this family is a highly homologous 120 amino acid region termed an RGS domain. Currently there are more than 30 mammalian proteins or partial sequences that contain a putative RGS domain (14). Some RGS family members are relatively low molecular weight proteins composed primarily of the RGS domain flanked by short amino and carboxy-terminal regions, and others are quite large with putative functional domains which implicate them in scaffolding reactions [e.g., pleckstrin homology (PH) domain, Db1 domain that is homologous to the db1 proto-oncogene domain, DEP domain that is present in dishevelled, eg1-10, and pleckstrin domain, and G protein γ-like (GGL) subunit domain] (14).

RGS proteins are thought to regulate GPCR signaling by interacting with the alpha subunits of heterotrimeric GTP-binding proteins. Heterotrimeric G proteins act as molecular switches in GPCR-mediated signal transduction controlling the rate and extent of activation of the effector (for a review of heterotrimeric G proteins, see 15). Stimulation of receptors by agonists leads to rapid dissociation of GDP from the α subunit and exchange for GTP. While complexed with GTP, the alpha subunit is held in its active state and results in interaction with downstream effectors. Hydrolysis of GTP returns the alpha subunit to its GDP-bound or inactive state. RGS proteins attenuate signaling through GPCRs by acting as GTPase activating proteins (GAPs) (13, 16, 17). By accelerating GTP hydrolysis, the RGS protein limits the time the G_{α} subunit spends in its active state. Structural studies indicate that RGSs bind to the transition state of the alpha subunit thereby stabilizing it and accelerating GTP hydrolysis (18,19). The transition state of the G_{α} subunit can be mimicked in vitro via treatment of the alpha subunits with aluminum tetrafluoride (AlF₄⁻). So far RGSs have been identified which interact with and activate members of the G_{αi} family (G_{αi1}, G_{αi2}, G_{αi3}, G_{αz}, G_{αo}, G_{αt}), G_{αq/11}, and G_{α12/13} but not G_{αs} (14). In addition to their GAP activity, RGSs may also block signaling by acting as effector antagonists (20, 21). RGS proteins have been identified in a variety of cell types and tissues which profoundly alter many GPCR-stimulated intracellular effectors, including regulation of adenylyl cyclase (22), MAP kinase activity (10, 20), inositol trisphosphate and Ca²⁺ signaling (21-23), K⁺ channel conductances (24) and visual signal transduction (25, 26).

Since several of these signaling cascades are involved in platelet activation, it is likely that one or more members of the RGS superfamily might be present in platelets and be responsible for regulating signaling pathways critical for platelet activation. In platelets, receptors for ADP, thromboxane A₂ and thrombin couple to heterotrimeric GTP-binding proteins that transduce the signals to intracellular effectors, resulting in inhibition of adenylyl cyclase, activation of phospholipase C and mobilization of intracellular calcium (5). All three of these receptors appear to couple to one or more alpha subunits in platelets. For ADP there are at least two receptors on the platelet surface coupled to heterotrimeric G proteins. The putative P2T_{AC} is coupled to G_{αi} (27), and the P2Y1 is coupled to G_{αq/11}, (28). Thrombin receptors appear to couple to G_{αi}, G_{αq/11} and G_{α12/13} family members (29-31). Thromboxane A₂ receptors have been shown to interact with both G_{αq/11}, (32-35), G_{α13} (31, 36) and G_{αi} (33) isoforms. More recently it has been shown that in platelets, concomitant activation of both G_{αi}- and G_{αq}-linked pathways are critical for platelet aggregation by ADP (37, 38). Not surprisingly, G_{αq} has been implicated as an integral regulator of hemostasis in mice, since G_{αq} knock-out mice exhibit profound bleeding tendencies, and die perinatally from hemorrhage (39).

The citation of any reference herein should not be construed as an admission that such reference is available as “Prior Art” to the instant application.

### SUMMARY OF TEE INVENTION

The present invention relates to nucleic acids encoding a novel Regulator of G protein Signaling (RGS) protein, RGS18. Thus, a first subject of the invention is a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The invention also relates to a nucleic acid comprising at least 8 consecutive nucleotides of a nucleic acid according to the invention. Preferably, a nucleic acid according to the invention will comprise 10, 12, 15, 18, 20 to 25, 35, 40, 50, 70, 80, 100, 200, 500, 1000, or 1500 consecutive nucleotides of a nucleic acid according to the invention.

The invention also relates to a nucleic acid having at least 80% nucleotide identity with a nucleic acid according to the invention. The invention also relates to a nucleic acid having at least 85%, preferably 90%, more preferably 95% and still more preferably 98% nucleotide identity with a nucleic acid according to the invention.

The invention also relates to a nucleic acid hybridizing, under high stringency conditions, with a polynucleotide sequence of a nucleic acid of the invention.

The present invention also relates to nucleic acids encoding a polypeptide comprising the novel RGS18 domain. Thus, a second subject of the invention relates to a nucleic acid comprising a polynucleotide sequence of a) either of SEQ ID NOs: 18 or 19, or of a complementary polynucleotide sequence, b) nucleotides 163-870 of SEQ ID NO: 19, or of a complementary polynucleotide sequence, or c) nucleotides 418-768 of SEQ ID NO: 19, or of a complementary polynucleotide sequence.

The invention also relates to nucleic acids, particularly cDNA molecules, which encode the full length human RGS18 protein. The present invention also relates to a cDNA molecule that encodes the novel full length RGS18 protein. Thus, the invention relates to a nucleic acid comprising a polynucleotide sequence of a) either of SEQ ID NOs: 18 or 19, or of a complementary polynucleotide sequence, or b) nucleotides 163-870 of SEQ ID NO: 19, or of a complementary polynucleotide sequence.

According to the invention, a nucleic acid comprising a polynucleotide sequence of either SEQ ID NOs: 18 or 19 encodes a full length RGS18 domain polypeptide of 235 amino acids comprising the amino acid sequence of SEQ ID NO: 20. The present invention also relates to a nucleic acid that encodes a polypeptide comprising an amino acid sequence of amino acids 86-202 of SEQ ID NO: 20. In another preferred embodiment, a nucleic acid according to the invention encodes a polypeptide comprising an amino acid sequence of SEQ ID NO: 20.

The present invention also relates to polypeptides comprising the novel RGS18 domain according to the invention. In a preferred embodiment, a polypeptide according to the invention comprises an amino acid sequence of amino acids 86-202 of SEQ ID NO: 20. In another preferred embodiment, the polypeptide according to the invention comprises an amino acid sequence of SEQ ID NO: 20.

The invention also relates to a polypeptide comprising an amino acid sequence having at least 80% amino acid identity with a polypeptide comprising an amino acid sequence of a) either SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

In a specific embodiment, the invention relates to a polypeptide having at least 85%, preferably 90%, more preferably 95% and still more preferably 98% amino acid identity with a polypeptide according to the invention.

The present invention also provides nucleotide probes and primers that hybridize with a nucleic acid sequence of a nucleic acid according to the invention. The nucleotide probes or primers according to the invention comprise at least 8 consecutive nucleotides of a nucleic acid comprising a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence. Preferably, nucleotide probes or primers according to the invention will have a length of 10, 12, 15, 18, 20 to 25, 35, 40, 50, 70, 80, 100, 200, 500, 1000, or 1500 consecutive nucleotides of a nucleic acid according to the invention.

The present invention also relates to a method of amplifying a nucleic acid according to the invention contained in a sample, wherein said method comprises the steps of:

a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and

b) detecting the amplified nucleic acids.

The present invention also relates to a method of detecting the presence of a nucleic acid according to the invention in a sample, wherein said method comprises the steps of:

1) bringing one or more nucleotide probes according to the invention into contact with the sample to be tested;

2) detecting the complex which may have formed between the probe(s) and the nucleic acid present in the sample.

Another subject of the invention is a box or kit for amplifying all or part of a nucleic acid according to the invention, wherein said box or kit comprises:

1) a pair of nucleotide primers in accordance with the invention, whose hybridization position is located respectively on the 5′ side and 3′ side of the target nucleic acid whose amplification is sought; and optionally,

2) reagents necessary for an amplification reaction.

The invention also relates to a box or kit for detecting the presence of a nucleic acid according to the invention in a sample, said box or kit comprising:

a) one or more nucleotide probes according to the invention; and

b) where appropriate, reagents necessary for a hybridization reaction.

The invention also relates to a recombinant vector comprising a nucleic acid according to the invention.

The present invention also relates to a defective recombinant virus comprising a nucleic acid encoding an RGS18 polypeptide. In a preferred embodiment, the defective recombinant virus comprises a cDNA molecule that encodes an RGS18 polypeptide. In another preferred embodiment of the invention, the defective recombinant virus comprises a gDNA molecule that encodes an RGS18 polypeptide. Preferably, the encoded RGS18 polypeptide comprises amino acids 86-166 of SEQ ID NO: 20. More preferably, the encoded RGS18 polypeptide comprises amino acids 86-202 of SEQ ID NO: 20. Even more preferably, the encoded RGS18 polypeptide comprises an amino acid sequence of SEQ ID NO: 20.

In another preferred embodiment, the invention relates to a defective recombinant virus comprising a nucleic acid encoding an RGS18 protein under the control of a promoter chosen from Rous sarcoma virus long terminal repeat (RSV-LTR) or the cytomegalovirus (CMV) early promoter.

The present invention also relates to a composition comprising a nucleic acid encoding a polypeptide according to the invention, wherein the nucleic acid is placed under the control of appropriate regulatory elements.

The invention also relates to the use of a nucleic acid, polypeptide, or recombinant vector according to the invention for the manufacture of a medicament intended for the treatment and/or prevention of a disorder or condition associated with platelet activation dysfunction.

The invention also relates to the use of a nucleic acid, polypeptide, or recombinant vector according to the invention for the preparation of a pharmaceutical composition intended for the treatment and/or for the prevention of disorders or conditions associated with platelet activation dysfunction.

Thus, the present invention also relates to a pharmaceutical composition comprising a nucleic acid, polypeptide, or recombinant vector according to the invention, combined with one or more physiologically compatible vehicles and/or excipients.

The present invention also relates to the use of cells genetically modified ex vivo with a nucleic acid or recombinant vector according to the invention, or of cells producing a recombinant vector, wherein the cells are implanted in the body, to allow a prolonged and effective expression in vivo of a biologically active RGS18 polypeptide.

Thus, the invention also relates to the use of a recombinant host cell according to the invention, comprising a nucleic acid encoding an RGS18 polypeptide according to the invention for the manufacture of a medicament intended for the prevention of, or more particularly, for the treatment of subjects affected by a disorder or condition associated with platelet activation dysfunction.

The present invention also relates to the use of a recombinant host cell according to the invention, for the preparation of a pharmaceutical composition for the treatment and/or prevention of pathologies linked to a disorder or condition associated with platelet activation dysfunction.

The invention relates to the use of a defective recombinant virus according to the invention for the preparation of a pharmaceutical composition intended for the treatment and/or for the prevention of a disorder or condition associated with platelet activation dysfunction. Thus, the present invention also relates to a pharmaceutical composition comprising a defective recombinant virus according to the invention, combined with one or more physiologically compatible vehicles and/or excipients.

The present invention also relates to the use of cells genetically modified ex vivo with a recombinant defective virus according to the invention, or of cells producing such viruses, implanted in the body, allowing a prolonged and effective expression in vivo of a biologically active RSG18 protein. A specific embodiment of the invention is an isolated mammalian cell infected with one or more defective recombinant viruses according to the invention.

Another subject of the invention relates to an implant comprising isolated mammalian cells infected with one or more defective recombinant viruses according to the invention or cells producing recombinant viruses, and an extracellular matrix. More particularly, in the implants of the invention, the extracellular matrix comprises a gelling compound and optionally, a support allowing the anchorage of the cells.

The invention also relates to an isolated recombinant host cell comprising a nucleic acid of the invention.

According to another aspect, the invention also relates to an isolated recombinant host cell comprising a recombinant vector according to the invention. Therefore, the invention also relates to a recombinant host cell comprising a recombinant vector comprising a nucleic acid of the invention.

The invention also relates to a method for the production of a polypeptide according to the invention, wherein said method comprises the steps of:

a) inserting a nucleic acid encoding said polypeptide into an appropriate vector;

b) culturing, in an appropriate culture medium, a previously transformed host cell or transfecting a host cell with the recombinant vector of step a);

c) recovering the conditioned culture medium or lysing the host cell, for example by sonication or by osmotic shock;

d) separating and purifying said polypeptide from said culture medium or alternatively from the cell lysates obtained in step c); and

e) where appropriate, characterizing the recombinant polypeptide produced.

The present invention also relates to antibodies directed against a polypeptide according to the invention.

Thus, another subject of the invention is a method of detecting the presence of a polypeptide according to the invention in a sample, wherein said method comprises the steps of:

a) bringing the sample to be tested into contact with an antibody directed against a polypeptide according to the invention, and

b) detecting the antigen/antibody complex formed.

The invention also relates to a box or kit for diagnosis or for detecting the presence of a polypeptide in accordance with the invention in a sample, said box comprising:

a) an antibody directed against a polypeptide according to the invention, and

b) a reagent allowing the detection of the antigen/antibody complex formed.

The present invention also relates to a new therapeutic approach for the treatment of pathologies linked to a disorder or condition associated with platelet activation dysfunction, comprising transferring and expressing in vivo a nucleic acid, recombinant vector, or recombinant defective virus according to the invention. Specifically, the present invention provides a new therapeutic approach for the treatment and/or prevention of a disorder or condition associated with platelet activation dysfunction.

According to yet another aspect, the subject of the invention is also a preventive or curative therapeutic method of treating diseases caused by abnormal platelet activation, such a method comprising a step in which there is administered to a patient a therapeutically effective quantity of an RGS18 polypeptide according to the invention in said patient, said polypeptide being, where appropriate, combined with one or more physiologically compatible vehicles and/or excipients.

The invention also relates to methods for the detection of activators or inhibitors of RGS18 protein and RGS18 domain comprising polypeptides.

The invention also provides methods for screening small molecules and compounds that act on the RGS18 protein to identify agonists and antagonists of RGS18 polypeptide that can improve, reduce, or inhibit platelet activation from a therapeutic point of view. These methods are useful to identify small molecules and compounds for therapeutic use in the treatment of diseases due to a deficiency in platelet activation.

Therefore, the invention also relates to the use of a polypeptide according to the invention or a cell expressing a polypeptide according to the invention, for screening active ingredients for the prevention or treatment of a disorder or condition associated with platelet activation dysfunction. The invention also relates to a method of screening a compound or small molecule that functions as an agonist or antagonist of an RGS18 polypeptide.

### BRIEF DESCRIPTION OF THE DRAWINGS

FIG. 1. Cloning and Sequence information of a Full-length cDNA for a novel RGS, RGS18. Panel A, Schematic representation of the full-length cloning of the novel platelet RGS. A schematic of the cDNA for RGS18 is depicted on the bottom, with the boxed region representing the open predicted reading frame and the single lines the 5′ and 3′ untranslated regions. The relative locations of the initial RT-PCR product, the Incyte EST cDNA (clone 435706H1) and the 5′ RACE amplification product are shown above. Panel B, Nucleotide and deduced amino acid sequence of RGS18. The 5′ and 3′ untranslated regions are given in lower case letters, the predicted amino acid sequence in single letter abbreviations in uppercase letters. The initial RT-PCR product from platelet RNA is shown in bold-face type. The 5′ and 3′ ends of the Incyte EST cDNA are depicted by the diamonds “♦.” The oligonucleotide sequence of the primer in the far 3′ untranslated region used for 5′ RACE is underlined. The putative CAAX motif is noted by the double-underline ( [symbol] ) and the cAMP/cGMP-dependent protein kinase consensus site is noted by the triple underline (=).

FIG. 2. Alignment of RGS18 with other RGS family members. The predicted amino acid sequence of RGS18 was aligned with six other RGS protein sequences using the PILEUP program of GCG. Homology between RGS18 and these other RGS protein sequences is depicted by the shading. Amino acids which are conserved between RGS18 and at least two other RGSs are shaded. The solid line above the sequence indicates the conserved RGS domain which was amplified by PCR. The asterisks (*) denote the location of the two amino acids which are conserved in members of Family B. Down arrows (↑), depict amino acids in RGS4 which are predicted to contact G_{α} subunits. The boxed regions indicate the peptide sequences which were synthesized for production of peptide-directed antisera.

FIG. 3. Tissue distribution of RGS18 by Northern Blotting. Panel A, a Northern Blot of 10 mg of total RNA from human platelets, human leukocytes, DAMI, HEL, and MEG-01 cells probed with a 3′ untranslated region probe of RGS18 as described in Experimental Procedures. This blot was exposed to Kodak BioMax MR film for 6 hours at −70° C. Panel B, Hybridization of a Human Multiple Tissue Northern with the same RGS18 probe. This blot was exposed to Kodak BioMax film for 6 days at −70° C. Migration of molecular weight standards on each gel is shown on the left. After removal of the probe, each blot was hybridized with a β-actin probe for normalization, shown below the corresponding blot.

FIG. 4. Western blotting of platelet, leukocyte and megakaryocyte cell line lysates. Panel A, Specificity of anti-RGS-18 antisera. Nitrocellulose strips containing 50 mg of platelet lysate run on 15% SDS-PAGE were incubated with a 1:500 dilution of antisera 3NRGS-12 or a 1:1000 dilution of 5NRGS-13. An immunoreactive band that migrates at ˜30 kDa is detected by both antisera (first lane for each blot). Identical strips were also probed with antisera which had been preincubated with the corresponding immunizing peptide or with an unrelated peptide (in these studies 5NRGS-peptide was used for antisera #12 and 3NRGS-peptide for antisera #13). Migration of the 30 kDa molecular weight standard is shown on the left, migration of RGS18 on the right. Panel B, Detection of RGS18 expression in platelets, leukocytes and the megakaryocytic cell lines. Lysates (50 mg) from human platelets, leukocytes, DAMI HEL and MEG-01 cells were run on 15% SDS-PAGE, transferred to nitrocellulose and blotted with antibodies against RGS18 and RGS10 (Santa Cruz Biotechnology, Santa Cruz, Calif.). The top blot depicts reactivity of each lysate with the anti-RGS18 antisera (5NRGS-#13) and the bottom blot with the anti-RGS10 antisera. As seen above, RGS18 co-migrates with 30 kDa MW marker. RGS10, a much smaller protein, migrates close to the 21 kDa MW marker.

FIG. 5. Determination of the Ga subunit specificity of RGS18. Platelet lysates were treated with GDP or GDP+AlF₄⁻ as indicated and incubated with GST-RGS18 coupled to Sepharose 4B as described in Experimental Procedures. Bound proteins were subjected to 12% SDS-PAGE and transferred to nitrocellulose and detected with antisera against G_{αi1/αi2}, G_{αo/i3}, G_{αq/11}, G_{αz}, G_{α12} or G_{αs}. The lane labeled “Lysate” is 35 mg or 7.7% of the input lysate in each of the reactions run alongside to show reactivity of each antisera with platelet lysate.

### DETAILED DESCRIPTION OF THE INVENTION

General Definitions

The present invention contemplates isolation of a gene encoding an RGS18 polypeptide of the invention, including a full length, or naturally occurring form of RGS18, and any antigenic fragments thereof from any animal, particularly mammalian or avian, and more particularly human, source.

In accordance with the present invention there may be employed conventional molecular biology, microbiology, and recombinant DNA techniques within the skill of the art. Such techniques are explained fully in the literature. See, e.g., references 40-47.

Therefore, if appearing herein, the following terms shall have the definitions set out below.

As used herein, the term “gene” refers to an assembly of nucleotides that encode a polypeptide, and includes cDNA and genomic DNA nucleic acids.

The term “isolated” for the purposes of the present invention designates a biological material (nucleic acid or protein) that has been removed from its original environment (the environment in which it is naturally present).

For example, a polynucleotide present in the natural state in a plant or an animal is not isolated. The same polynucleotide separated from the adjacent nucleic acids in which it is naturally inserted in the genome of the plant or animal is considered as being “isolated”.

Such a polynucleotide may be included in a vector and/or such a polynucleotide may be included in a composition and remains nevertheless in the isolated state because of the fact that the vector or the composition does not constitute its natural environment,

The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

For the purposes of the present description, the expression “nucleotide sequence” may be used to designate either a polynucleotide or a nucleic acid. The expression “nucleotide sequence” covers the genetic material itself and is therefore not restricted to the information relating to its sequence.

The terms “nucleic acid”, “polynucleotide”, “oligonucleotide” or “nucleotide sequence” cover RNA, DNA, gDNA or cDNA sequences or alternatively RNA/DNA hybrid sequences of more than one nucleotide, either in the single-stranded form or in the duplex, double-stranded form.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA. The sequence of nucleotides that encodes a protein is called the sense sequence or coding sequence.

The term “nucleotide” designates both the natural nucleotides (A, T, G, C) as well as the modified nucleotides that comprise at least one modification such as (1) an analog of a purine, (2) an analog of a pyrimidine, or (3) an analogous sugar, examples of such modified nucleotides being described, for example, in the PCT application No. WO 95/04064.

For the purposes of the present invention, a first polynucleotide is considered as being “complementary” to a second polynucleotide when each base of the first nucleotide is paired with the complementary base of the second polynucleotide whose orientation is reversed. The complementary bases are A and T (or A and U), or C and G.

“Heterologous” DNA refers to DNA not naturally located in the cell, or in a chromosomal site of the cell. Preferably, the heterologous DNA includes a gene foreign to the cell.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (48). Such proteins (and their encoding genes) have sequence homology, as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see 48). However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and more preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., 41, 43, and 49.

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program.

The “percentage identity” between two nucleotide or amino acid sequences, for the purposes of the present invention, may be determined by comparing two sequences aligned optimally, through a window for comparison.

The portion of the nucleotide or polypeptide sequence in the window for comparison may thus comprise additions or deletions (for example “gaps”) relative to the reference sequence (which does not comprise these additions or these deletions) so as to obtain an optimum alignment of the two sequences.

The percentage is calculated by determining the number of positions at which an identical nucleic base or an identical amino acid residue is observed for the two sequences (nucleic or peptide) compared, and then by dividing the number of positions at which there is identity between the two bases or amino acid residues by the total number of positions in the window for comparison, and then multiplying the result by 100 in order to obtain the percentage sequence identity.

The optimum sequence alignment for the comparison may be achieved using a computer with the aid of known algorithms contained in the package from the company WISCONSIN GENETICS SOFTWARE PACKAGE, GENETICS COMPUTER GROUP (GCG), 575 Science Doctor, Madison, Wis.

By way of illustration, it will be possible to produce the percentage sequence identity with the aid of the BLAST software (versions BLAST 1.4.9 of March 1996, BLAST 2.0.4 of February 1998 and BLAST 2.0.6 of September 1998), using exclusively the default parameters (50, 51). Blast searches for sequences similar/homologous to a reference “request” sequence, with the aid of the Altschul et al. algorithm. The request sequence and the databases used may be of the peptide or nucleic types, any combination being possible.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A gene encoding an RGS18 polypeptide of the invention, whether genomic DNA or cDNA, can be isolated from any source, particularly from a human cDNA or genomic library. Methods for obtaining genes are well known in the art, as described above (see, e.g. 40).

Accordingly, any animal cell potentially can serve as the nucleic acid source for the molecular cloning of an RSG18 gene. The DNA may be obtained by standard procedures known in the art from cloned DNA (e.g., a DNA “library”), and preferably is obtained from a cDNA library prepared from tissues with high level expression of the protein, by chemical synthesis, by cDNA cloning, or by the cloning of genomic DNA, or fragments thereof, purified from the desired cell (See, for example, 40, 41). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired RGS18 gene may be accomplished in a number of ways. For example, if an amount of a portion of a RSG18 gene or its specific RNA, or a fragment thereof, is available and can be purified and labeled, the generated DNA fragments may be screened by nucleic acid hybridization to the labeled probe (52, 53). For example, a set of oligonucleotides corresponding to the partial amino acid sequence information obtained for the RGS18 protein can be prepared and used as probes for DNA encoding RGS18, as was done in a specific example, infra, or as primers for cDNA or mRNA (e.g., in combination with a poly-T primer for RT-PCR). Preferably, a fragment is selected that is highly unique to an RGS18 nucleic acid or polypeptide of the invention. Those DNA fragments with substantial homology to the probe will hybridize. As noted above, the greater the degree of homology, the more stringent hybridization conditions can be used. In a specific embodiment, stringency hybridization conditions are used to identify a homologous RGS18 gene.

Further selection can be carried out on the basis of the properties of the gene, e.g., if the gene encodes a protein product having the isoelectric, electrophoretic, amino acid composition, or partial amino acid sequence of an RGS18 protein as disclosed herein. Thus, the presence of the gene may be detected by assays based on the physical, chemical, or immunological properties of its expressed product. For example, cDNA clones, or DNA clones which hybrid-select the proper mRNAs, can be selected which produce a protein that, e.g., has similar or identical electrophoretic migration, isoelectric focusing or non-equilibrium pH gel electrophoresis behavior, proteolytic digestion maps, or antigenic properties as known for RGS18.

An RGS18 gene of the invention can also be identified by mRNA selection, i.e., by nucleic acid hybridization followed by in vitro translation. In this procedure, nucleotide fragments are used to isolate complementary mRNAs by hybridization. Such DNA fragments may represent available, purified RGS18 DNA, or may be synthetic oligonucleotides designed from the partial amino acid sequence information. Immunoprecipitation analysis or functional assays (e.g. tyrosine phosphatase activity) of the in vitro translation products of the products of the isolated mRNAs identifies the mRNA and, therefore, the complementary DNA fragments, that contain the desired sequences. In addition, specific mRNAs may be selected by adsorption of polysomes isolated from cells to immobilized antibodies specifically directed against an RGS18 polypeptide of the invention.

A radiolabeled RGS18 cDNA can be synthesized using the selected mRNA (from the adsorbed polysomes) as a template. The radiolabeled mRNA or cDNA may then be used as a probe to identify homologous RGS18 DNA fragments from among other genomic DNA fragments.

“Variant” of a nucleic acid according to the invention will be understood to mean a nucleic acid that differs by one or more bases relative to the reference polynucleotide. A variant nucleic acid may be of natural origin, such as an allelic variant that exists naturally, or it may also be a non-natural variant obtained, for example, by mutagenic techniques.

In general, the differences between the reference (generally, wild-type) nucleic acid and the variant nucleic acid are small such that the nucleotide sequences of the reference nucleic acid and of the variant nucleic acid are very similar and, in many regions, identical. The nucleotide modifications present in a variant nucleic acid may be silent, which means that they do not alter the amino acid sequences encoded by said variant nucleic acid.

However, the changes in nucleotides in a variant nucleic acid may also result in substitutions, additions or deletions in the polypeptide encoded by the variant nucleic acid in relation to the polypeptides encoded by the reference nucleic acid. In addition, nucleotide modifications in the coding regions may produce conservative or non-conservative substitutions in the amino acid sequence of the polypeptide.

Preferably, the variant nucleic acids according to the invention encode polypeptides that substantially conserve the same function or biological activity as the polypeptide of the reference nucleic acid or alternatively the capacity to be recognized by antibodies directed against the polypeptides encoded by the initial reference nucleic acid.

Some variant nucleic acids will thus encode mutated forms of the polypeptides whose systematic study will make it possible to deduce structure-activity relationships of the proteins in question. Knowledge of these variants in relation to the disease studied is essential since it makes it possible to understand the molecular cause of the pathology.

“Fragment” will be understood to mean a nucleotide sequence of reduced length relative to the reference nucleic acid and comprising, over the common portion, a nucleotide sequence identical to the reference nucleic acid. Such a nucleic acid “fragment” according to the invention may be, where appropriate, included in a larger polynucleotide of which it is a constituent. Such fragments comprise, or alternatively consist of, oligonucleotides ranging in length from at least 8, 10, 12, 15, 18, 20 to 25, 30, 40, 50, 70, 80, 100, 200, 500, 1000 or 1500 consecutive nucleotides of a nucleic acid according to the invention.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see 40). The conditions of temperature and ionic strength determine the “stringency” of the hybridization. For preliminary screening for homologous nucleic acids, low stringency hybridization conditions, corresponding to a Tₘ of 550, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see 40, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). Preferably a minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

“High stringency hybridization conditions” for the purposes of the present invention will be understood to mean the following conditions:

1—Membrane Competition and Prehybridization:

Mix: 40 μl salmon sperm DNA (10 mg/ml)+

+40 μl human placental DNA (10 mg/ml)

Denature for 5 minutes at 96° C., then immerse the mixture in ice.

Remove the 2×SSC and pour 4 ml of formamide mix in the hybridization tube containing the membranes.

Add the mixture of the two denatured DNAs.

Incubation at 42° C. for 5 to 6 hours, with rotation.

2—Labeled Probe Competition:

Add to the labeled and purified probe 10 to 50 μl Cot I DNA, depending on the quantity of repeats.

Denature for 7 to 10 minutes at 95° C.

Incubate at 65° C. for 2 to 5 hours.

3—Hybridization:

Remove the prehybridization mix.

Mix 40 μl salmon sperm DNA+40 μl human placental DNA; denature for 5 min at 96° C., then immerse in ice.

Add to the hybridization tube 4 ml of formamide mix, the mixture of the two DNAs and the denatured labeled probe/Cot I DNA.

Incubate 15 to 20 hours at 42° C., with rotation.

4—Washes and Exposure:

One wash at room temperature in 2×SSC, to rinse.

Twice 5 minutes at room temperature 2×SSC and 0.1% SDS at 65° C.

Twice 15 minutes at 65° C. 1×SSC and 0.1% SDS at 65° C.

Envelope the membranes in clear plastic wrap and expose.

The hybridization conditions described above are adapted to hybridization, under high stringency conditions, of a molecule of nucleic acid of varying length from 20 nucleotides to several hundreds of nucleotides. It goes without saying that the hybridization conditions described above may be adjusted as a function of the length of the nucleic acid whose hybridization is sought or of the type of labeling chosen, according to techniques known to one skilled in the art. Suitable hybridization conditions may, for example, be adjusted according to the teaching contained in references 43 or 47.

As used herein, the term “oligonucleotide” refers to a nucleic acid, generally of at least 15 nucleotides, that is hybridizable to a nucleic acid according to the invention. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid encoding an RGS18 polypeptide of the invention. In another embodiment, oligonucleotides (one or both of which may be labeled) can be used as PCR primers, either for cloning full length or a fragment of an RGS18 nucleic acid, or to detect the presence of nucleic acids encoding RGS18. In a further embodiment, an oligonucleotide of the invention can form a triple helix with an RGS18 DNA-molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence that is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

“Regulatory region” means a nucleic acid sequence that regulates the expression of a nucleic acid. A regulatory region may include sequences that are naturally responsible for expressing a particular nucleic acid (a homologous region) or may include sequences of a different origin (responsible for expressing different proteins or even synthetic proteins). In particular, the sequences can be sequences of eukaryotic or viral genes or derived sequences that stimulate or repress transcription of a gene in a specific or non-specific manner and in an inducible or non-inducible manner. Regulatory regions include origins of replication, RNA splice sites, enhancers, transcriptional termination sequences, signal sequences that direct the polypeptide into the secretory pathways of the target cell, and promoters.

A regulatory region from a “heterologous source” is a regulatory region that is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

A “signal sequence” is included at the beginning of the coding sequence of a protein to be expressed on the surface of a cell. This sequence encodes a signal peptide, N-terminal to the mature polypeptide, which directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

A “polypeptide” is a polymeric compound comprised of covalently linked amino acid residues. Amino acids have the following general structure:

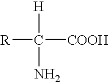

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

A “protein” is a polypeptide that plays a structural or functional role in a living cell.

The polypeptides and proteins of the invention may be glycosylated or unglycosylated.

“Homology” means similarity of sequence reflecting a common evolutionary origin. Polypeptides or proteins are said to have homology, or similarity, if a substantial number of their amino acids are either (1) identical, or (2) have a chemically similar R side chain. Nucleic acids are said to have homology if a substantial number of their nucleotides are identical.

“Isolated polypeptide” or “isolated protein” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

“Fragment” of a polypeptide according to the invention will be understood to mean a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of 10, 15, 20, 30 to 40, 50, 100, 200 or 300 amino acids.

“Variant” of a polypeptide according to the invention will be understood to mean mainly a polypeptide whose amino acid sequence contains one or more substitutions, additions or deletions of at least one amino acid residue, relative to the amino acid sequence of the reference polypeptide, it being understood that the amino acid substitutions may be either conservative or non-conservative.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. Variants also include a related protein having substantially the same biological activity, but obtained from a different species.

The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art.

If such allelic variations, analogues, fragments, derivatives, mutants, and modifications, including alternative mRNA splicing forms and alternative post-translational modification forms result in derivatives of the polypeptide which retain any of the biological properties of the polypeptide, they are intended to be included within the scope of this invention.

A “vector” is a replicon, such as plasmid, virus, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

The present invention also relates to cloning vectors containing genes encoding analogs and derivatives of an RGS18 polypeptide of the invention, that have the same or homologous functional activity as that RGS18 polypeptide, and homologs thereof from other species. The production and use of derivatives and analogs related to RGS18 are within the scope of the present invention. In a specific embodiment, the derivative or analog is functionally active, i.e., capable of exhibiting one or more functional activities associated with a full-length, wild-type RGS18 polypeptide of the invention.

RGS18 derivatives can be made by altering encoding nucleic acid sequences by substitutions, additions or deletions that provide for functionally equivalent molecules. Preferably, derivatives are made that have enhanced or increased functional activity relative to native RGS18.

Due to the degeneracy of nucleotide coding sequences, other DNA sequences which encode substantially the same amino acid sequence as an RGS18 gene may be used in the practice of the present invention. These include but are not limited to allelic genes, homologous genes from other species, and nucleotide sequences comprising all or portions of RGS18 genes that are altered by the substitution of different codons that encode the same amino acid residue within the sequence, thus producing a silent change. Likewise, the RGS18 derivatives of the invention include, but are not limited to, those containing, as a primary amino acid sequence, all or part of the amino acid sequence of an RGS18 protein including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free CONH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces b-turns in the protein's structure.

The genes encoding RGS18 derivatives and analogs of the invention can be produced by various methods known in the art. The manipulations which result in their production can occur at the gene or protein level. For example, the cloned RGS18 gene sequence can be modified by any of numerous strategies known in the art (40). The sequence can be cleaved at appropriate sites with restriction endonuclease(s), followed by further enzymatic modification if desired, isolated, and ligated in vitro. In the production of the gene encoding a derivative or analog of RGS18, care should be taken to ensure that the modified gene remains within the same translational reading frame as the RGS18 gene, uninterrupted by translational stop signals, in the gene region where the desired activity is encoded.

Additionally, the RGS18-encoding nucleic acids can be mutated in vitro or in vivo, to create and/or destroy translation, initiation, and/or termination sequences, or to create variations in coding regions and/or form new restriction endonuclease sites or destroy preexisting ones, to facilitate further in vitro modification. Preferably, such mutations enhance the functional activity of the mutated RGS18 gene product. Any technique for mutagenesis known in the art can be used, including but not limited to, in vitro site-directed mutagenesis (54-58) use of TAB® linkers (Pharmacia), etc. PCR techniques are preferred for site directed mutagenesis (see 59).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, Escherichia coli, bacteriophages such as lambda derivatives, or plasmids such as pBR322 derivatives or pUC plasmid derivatives, e.g. pGEX vectors, pmal-c, pFLAG, etc. The insertion into a cloning vector can, for example, be accomplished by ligating the DNA fragment into a cloning vector that has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., Escherichia coli, and facile purification for subsequent insertion into an appropriate expression cell line, if such is desired. For example, a shuttle vector, which is a vector that can replicate in more than one type of organism, can be prepared for replication in both Escherichia coli and Saccharomyces cerevisiae by linking sequences from an Escherichia coli plasmid with sequences from the yeast 2 m plasmid.

In an alternative method, the desired gene may be identified and isolated after insertion into a suitable cloning vector in a “shot gun” approach. Enrichment for the desired gene, for example, by size fractionation, can be done before insertion into the cloning vector.

The nucleotide sequence coding for an RGS18 polypeptide or antigenic fragment, derivative or analog thereof, or a functionally active derivative, including a chimeric protein, thereof, can be inserted into an appropriate expression vector, i.e., a vector which contains the necessary elements for the transcription and translation of the inserted protein-coding sequence. Such elements are termed herein a “promoter.” Thus, the nucleic acid encoding an RGS18 polypeptide of the invention is operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by a native gene encoding RGS18 and/or its flanking regions.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant RGS18 protein of the invention, or functional fragment, derivative, chimeric construct, or analog thereof, may be expressed chromosomally, after integration of the coding sequence by recombination. In this regard, any of a number of amplification systems may be used to achieve high levels of stable gene expression (40).

The cell into which the recombinant vector comprising the nucleic acid encoding an RGS18 polypeptide according to the invention is cultured in an appropriate cell culture medium under conditions that provide for expression of the RGS18 polypeptide by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of an RGS18 polypeptide may be controlled by any promoter/enhancer element known in the art, but these regulatory elements must be functional in the host selected for expression. Promoters which may be used to control RGS18 gene expression include, but are not limited to, the SV40 early promoter region (60), the promoter contained in the 3′ long terminal repeat of Rous sarcoma virus (61), the herpes thymidine kinase promoter (62), the regulatory sequences of the metallothionein gene (63); prokaryotic expression vectors such as the b-lactamase promoter (64), or the tac promoter (65); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (66-68); insulin gene control region which is active in pancreatic beta cells (69), immunoglobulin gene control region which is active in lymphoid cells (70-72), mouse mammary tumor virus control region which is active in testicular, breast, lymphoid and mast cells (73), albumin gene control region which is active in liver (74), alpha-fetoprotein gene control region which is active in liver (75, 76), alpha 1-antitrypsin gene control region which is active in the liver (77) beta-globin gene control region which is active in myeloid cells (78, 79), myelin basic protein gene control region which is active in oligodendrocyte cells in the brain (80), myosin light chain-2 gene control region which is active in skeletal muscle (81), and gonadotropic releasing hormone gene control region which is active in the hypothalamus (82).

Expression vectors comprising a nucleic acid encoding an RGS18 polypeptide of the invention can be identified by four general approaches: (a) polymerase chain reaction (PCR) amplification of the desired plasmid DNA or specific mRNA, (b) nucleic acid hybridization, (c) presence or absence of selection marker gene functions, (d) analyses with appropriate restriction endonucleases, and. (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding an RGS18 polypeptide is inserted within the “selection marker” gene sequence of the vector, recombinants comprising the RGS18 nucleic acid insert can be identified by the absence of the “selection marker” gene function. In the fourth approach, recombinant expression vectors are identified by digestion with appropriate restriction enzymes. In the fifth approach, recombinant expression vectors can be identified by assaying for the activity, biochemical, or immunological characteristics of the gene product expressed by the recombinant, provided that the expressed protein assumes a functionally active conformation.

A wide variety of host/expression vector combinations may be employed in expressing the nucleic acids of this invention. Useful expression vectors, for example, may consist of segments of chromosomal, non-chromosomal and synthetic DNA sequences. Suitable vectors include derivatives of SV40 and known bacterial plasmids, e.g., Escherichia coli plasmids col E1, pCR1, pBR322, pMal-C2, pET, pGEX (83), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2 m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BainHl cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedrin initiation codon; Invitrogen (195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR (84). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (84) for use according to the invention include but are not limited to pSC11 (SmaI cloning site, TK- and b-gal selection), pMJ601 (SalI, SmaI, AflI, NarI, BspMII, BamHI, ApaI, NheI, SacII, KpnI, and HindIII cloning site; TK- and b-gal selection), and pTKgptF1S (EcoRI, PstI, SalI, AccI, HindIII, SbaI, BamHI, and Hpa cloning site, TK or XPRT selection).

Yeast expression systems can also be used according to the invention to express an RGS18 polypeptide. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Different host cells have characteristic and specific mechanisms for the translational and post-translational processing and modification (e.g., glycosylation, cleavage [e.g., of signal sequence]) of proteins. Appropriate cell lines or host systems can be chosen to ensure the desired modification and processing of the foreign protein expressed. For example, expression in a bacterial system can be used to produce a non-glycosylated core protein product. However, the RGS18 protein expressed in bacteria may not be properly folded. Expression in yeast can produce a glycosylated product. Expression in eukaryotic cells can increase the likelihood of “native” glycosylation and folding of a heterologous protein. Moreover, expression in mammalian cells can provide a tool for reconstituting, or constituting, RGS18 activity. Furthermore, different vector/host expression systems may affect processing reactions, such as proteolytic cleavages, to a different extent.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., 85-87).

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

A recombinant marker protein expressed as an integral membrane protein can be isolated and purified by standard methods. Generally, the integral membrane protein can be obtained by lysing the membrane with detergents, such as but not limited to, sodium dodecyl sulfate (SDS), Triton X-100 polyoxyethylene ester, Ipagel/nonidet P40 (NP40) (octylphenoxy)-polyethoxyethanol, digoxin, sodium deoxycholate, and the like, including mixtures thereof. Solubilization can be enhanced by sonication of the suspension. Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Alternatively, a nucleic acid or vector according to the invention can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (88-90). The use of cationic lipids may promote encapsulation of negatively charged nucleic acids, and also promote fusion with negatively charged cell membranes (91). Particularly useful lipid compounds and compositions for transfer of nucleic acids are described in International Patent Publications WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting [89]. Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid (see U.S. Pat. Nos. 5,693,622, 5,589,466 and 5,580,859). Naked DNA vectors for gene therapy can be introduced into the desired host cells by methods known in the art, e.g., transfection, electroporation, microinjection, transduction, cell fusion, DEAE-dextran, calcium phosphate precipitation, use of a gene gun, or use of a DNA vector transporter (see, 85-87, 92). Receptor-mediated DNA delivery approaches can also be used (93, 94).

“Pharmaceutically acceptable vehicle or excipient” includes diluents and fillers which are pharmaceutically acceptable for method of administration, are sterile, and may be aqueous or oleaginous suspensions formulated using suitable dispersing or wetting agents and suspending agents. The particular pharmaceutically acceptable carrier and the ratio of active compound to carrier are determined by the solubility and chemical properties of the composition, the particular mode of administration, and standard pharmaceutical practice.

Any nucleic acid, polypeptide, vector, or host cell of the invention will preferably be introduced in vivo in a pharmaceutically acceptable vehicle or excipient. The phrase “pharmaceutically acceptable” refer, to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulator agency of the Federal or a state government or listed in the U.S. Pharmacopeia or other generally recognized pharmacopeia for use in animals, and more particularly in humans. The term “excipient” refers to a diluent, adjuvant, excipient, or vehicle with which the compound is administered. Such pharmaceutica carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as excipients, particularly for injectable solutions. Suitable pharmaceutical excipients are described in “Remington's Pharmaceutical Sciences” by E. W. Martin.

Naturally, the invention contemplates delivery of a vector that will express a therapeutically effective amount of an RGS18 polypeptide for gene therapy applications. The phrase “therapeutically effective amount” is used herein to mean an amount sufficient to reduce by at least about 15 percent, preferably by at least 50 percent, more preferably by at least 90 percent, and still more preferably prevent, a clinically significant deficit in the activity, function and response of the host. Alternatively, a therapeutically effective amount is sufficient to cause an improvement in a clinically significant condition in the host.

Nucleic Acids Encoding RGS18 Polypeptides

In an effort to better understand modulation of GPCR-mediated signaling in platelets, Applicants sought to identify Regulators of G protein signaling proteins (RGSs) that are present in human platelets and several megakaryocytic cell lines. Using degenerate oligonucleotides based on conserved regions of the highly homologous RGS domain, RT-PCR was performed using human platelet RNA, as well as RNA from several megakaryocytic cell lines. In addition to confirming the presence of several known RGS transcripts, a novel RGS domain containing transcript was found in platelet RNA. Northern blot analysis of multiple human tissues indicates that this novel transcript is most abundantly expressed in platelets compared to other tissues examined. This RGS transcript is abundantly expressed in platelets, with significantly lower expression in other tissues, primarily those of the hematopoetic system. This transcript is modestly expressed in three megakaryocyte cell lines and tissues of hematopoetic origin such as leukocytes, bone marrow and spleen with low level expression detected in other tissues as well. Full-length cloning of this novel RGS, which has been termed RGS18, demonstrates that this transcript encodes a 235 amino acid protein. RGS18 is most closely related to RGS5 (46% identity) and has ˜30˜40% identity to other RGS proteins. Peptide-directed antisera against RGS18 detect the expression of ˜30 kDa protein in platelet, leukocyte and megakaryocyte cell line lysates. In vitro RGS18 binds to endogenous G_{αi2}, G_{αi3} and G_{αq} but not G_{αz}, G_{αs} or G_{α12} from GDP+AlF₄⁻-treated platelet lysates. Since platelet aggregation requires activation of a receptor coupled to G_{αq} and/or one or more forms of G_{αi}, RGS18 may be responsible in part for regulation of pathways important to platelet activation.

The present invention relates to nucleic acids encoding a novel Regulator of G protein Signaling (RGS) protein, RGS18. Thus, a first subject of the invention is a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The invention also relates to a nucleic acid comprising at least 8 consecutive nucleotides of a polynucleotide sequence of a) nucleotides 1-169 of SEQ ID NO: 1, or of a complementary polynucleotide sequence, b) nucleotides 1-658 of SEQ ID NO: 19, or of a complementary polynucleotide sequence, c) nucleotides 163-658 of SEQ ID NO: 19, or of a complementary polynucleotide sequence, or d) nucleotides 418-658 of SEQ ID NO: 19, or of a complementary polynucleotide sequence.

Preferably, a nucleic acid according to the invention comprises at least 10, 12, 15, 18, 20 to 25, 35, 40, 50, 70, 80, 100, 200, or 500 consecutive nucleotides of a polynucleotide sequence of a) nucleotides 1-169 of SEQ ID NO: 11, or of a complementary polynucleotide sequence, b) nucleotides 1-658 of SEQ ID NO: 19, or of a complementary polynucleotide sequence, c) nucleotides 163-658 of SEQ ID NO: 19, or of a complementary polynucleotide sequence, or d) nucleotides 418-658 of SEQ ID NO: 19, or of a complementary polynucleotide sequence.

The invention also relates to a nucleic acid having at least 80% nucleotide identity with a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The invention also relates to a nucleic acid having at least 85%, preferably 90%, more preferably 95% and still more preferably 98% nucleotide identity with a nucleic acid comprising a polynucleotide sequence of a) any one of SEQ ID NOs: 11, 18, or 19, or of a complementary polynucleotide sequence, b) nucleotides 1-169 of SEQ ID NO: 1, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The invention also relates to a nucleic acid hybridizing, under high stringency conditions, with a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The present invention also relates to nucleic acids encoding a polypeptide comprising the novel RGS18 domain. Thus, a second subject of the invention relates to a nucleic acid comprising a polynucleotide sequence of a) either of SEQ ID NOs: 18 or 19, or of a complementary polynucleotide sequence, b) nucleotides 163-870 of SEQ ID NO: 19, or of a complementary polynucleotide sequence, or c) nucleotides 418-768 of SEQ ID NO: 19, or of a complementary polynucleotide sequence.

The invention also relates to nucleic acids, particularly cDNA molecules, which encode the full length human RGS18 protein. The present invention also relates to a cDNA molecule that encodes the novel full length RGS18 protein. Thus, the invention relates to a nucleic acid comprising a polynucleotide sequence of a) either of SEQ ID NOs: 18 or 19, or of a complementary polynucleotide sequence, or b) nucleotides 163-870 of SEQ ID NO: 19, or of a complementary polynucleotide sequence.

The invention also relates to a nucleic acid comprising a polynucleotide sequence as depicted in a) either one of SEQ ID NOs: 18 or 19, or a complementary polynucleotide sequence, or b) nucleotides 163-870 of SEQ ID NO: 19, or a complementary polynucleotide sequence.

According to the invention, a nucleic acid comprising a polynucleotide sequence of either SEQ ID NOs: 18 or 19 encodes a full length RGS18 domain polypeptide of 235 amino acids comprising the amino acid sequence of SEQ ID NO: 20.

The present invention also relates to a nucleic acid that encodes a polypeptide comprising the novel RGS18 domain. In a preferred embodiment, the nucleic acid encodes a polypeptide comprising an amino acid sequence of amino acids 86-202 of SEQ ID NO: 20. In another preferred embodiment, the nucleic acid encodes a polypeptide comprising an amino acid sequence of SEQ ID NO: 20.

RGS18 Polypeptides

The present invention also relates to polypeptides comprising the novel RGS18 domain according to the invention.

Thus, the present invention relates to a nucleic acid that encodes a polypeptide comprising an amino acid sequence of amino acids 86-202 of SEQ ID NO: 20.

In addition, the present invention also relates to a polypeptide comprising the novel RGS18 domain. In a preferred embodiment, a polypeptide according to the invention comprises an amino acid sequence of amino acids 86-202 of SEQ ID NO: 20. In another preferred embodiment, the polypeptide according to the invention comprises an amino acid sequence of SEQ ID NO: 20.

The invention also relates to a polypeptide comprising an amino acid sequence as depicted in SEQ ID NO: 20.

The invention also relates to a polypeptide comprising an amino acid sequence comprising amino acids 86-202 of SEQ ID NO: 20.

The invention also relates to a polypeptide comprising an amino acid sequence having at least 80% amino acid identity with a polypeptide comprising an amino acid sequence of a) either SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

The invention also relates to a polypeptide having at least 85%, preferably 90%, more preferably 95% and still more preferably 98% amino acid identity with a polypeptide comprising an amino acid sequence of a) either SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

Preferably, a polypeptide according to the invention will have a length of 15, 18 or 20 to 25, 35, 40, 50, 70, 80, 100 or 200 consecutive amino acids of a polypeptide according to the invention, in particular a polypeptide comprising an amino acid sequence of a) amino acids 1-58 of SEQ ID NO: 12, b) amino acids 1-166 of SEQ ID NO: 20, or c) amino acids 86-166 of SEQ ID NO: 20.

Alternatively, a polypeptide according to the invention will comprise a fragment having a length of 15, 18, 20, 25, 35, 40, 50, 100 or 200 consecutive amino acids of a polypeptide according to the invention, more particularly of a polypeptide comprising an amino acid sequence of a) amino acids 1-58 of SEQ ID NO: 12, b) amino acids 1-166 of SEQ ID NO: 20, or c) amino acids 86-166 of SEQ ID NO: 20.

Nucleotide Probes and Primers

Nucleotide probes and primers hybridizing with a nucleic acid (genomic DNA, messenger RNA, cDNA) according to the invention also form part of the invention.

The definition of a nucleotide probe or primer according to the invention therefore covers oligonucleotides which hybridize, under the high stringency hybridization conditions defined above, with a polynucleotide sequence of a nucleic acid according to the invention, or a complementary polynucleotide sequence.

According to the invention, nucleic acid fragments derived from a polynucleotide according to the invention are useful for the detection of the presence of at least one copy of a nucleotide sequence of an RGS18 nucleic acid or of a fragment or of a variant (containing a mutation or a polymorphism) thereof in a sample.

According to the invention, nucleic acid fragments derived from a nucleic acid comprising a polynucleotide sequence of any one of SEQ ID NOs: 11, 18, and 19, or of a complementary polynucleotide sequence, are useful for the detection of the presence of at least one copy of a nucleotide sequence of the RGS18 gene or of a fragment or of a variant (containing a mutation or a polymorphism) thereof in a sample. Thus, nucleotide probes and primers hybridizing with a nucleic acid sequence of a nucleic acid that encodes an RGS18 domain (genomic DNA, messenger RNA, cDNA), also form part of the invention.

The nucleotide probes or primers according to the invention comprise at least 8 consecutive nucleotides of a nucleic acid comprising a polynucleotide sequence of nucleotides a) 1-169 of SEQ ID NO: 11, or of a complementary polynucleotide sequence, b) 1-658 of SEQ BD NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence.

Preferably, nucleotide probes or primers according to the invention will have a length of 10, 12, 15, 18, 20 to 25, 35, 40, 50, 70, 80, 100, 200, 500, 1000, or 1500 consecutive nucleotides of a nucleic acid according to the invention, in particular of a nucleic acid comprising a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence.

Alternatively, a nucleotide probe or primer according to the invention will consist of and/or comprise a fragment having a length of 10, 12, 15, 18, 20, 25, 35, 40, 50, 100, 200, 500, 1000, or 1500 consecutive nucleotides of a nucleic acid according to the invention, more particularly of a nucleic acid comprising a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence.

The preferred probes and primers according to the invention comprise all or part of a polynucleotide sequence comprising a) any one of SEQ ID NOs: 9, 10, 14, 15, 16, 17, 30, 31, 32, 33, 34, 35, or 36, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-658 of SEQ ID NO: 19, or of a complementary polynucleotide sequence, or e) nucleotides 418-658 of SEQ ID NO: 19, or of a complementary polynucleotide sequence.

The nucleotide primers according to the invention may be used to amplify any one of the nucleic acids according to the invention, and more particularly a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

Alternatively, the nucleotide primers according to the invention may be used to amplify a nucleic acid fragment or variant of a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The definition of a nucleotide probe or primer according to the invention therefore covers oligonucleotides that hybridize, under the high stringency hybridization conditions defined above, with a nucleic acid according to the invention, or a complementary polynucleotide sequence.

According to a preferred embodiment, a nucleotide primer according to the invention comprises a nucleotide sequence of any one of SEQ ID NOs: 9, 10, 14, 15, 16, 17, 30, 31, 32, 33, 34, 35, or 36, or of a complementary nucleic acid sequence.

A nucleotide primer or probe according to the invention may be prepared by any suitable method well known to persons skilled in the art, including by cloning and action of restriction enzymes or by direct chemical synthesis according to techniques such as the phosphodiester method by Narang et al. (95) or by Brown et al. (96), the diethylphosphoramidite method by Beaucage et al. (97) or the technique on a solid support described in EU patent No. EP 0,707,592.

Each of the nucleic acids according to the invention, including the oligonucleotide probes and primers described above, may be labeled, if desired, by incorporating a marker which can be detected by spectroscopic, photochemical, biochemical, immunochemical or chemical means. For example, such markers may consist of radioactive isotopes (³²P, ³³P, ³H, ³⁵S), fluorescent molecules (5-bromodeoxyuridine, fluorescein, acetylaminofluorene, digoxigenin) or ligands such as biotin. The labeling of the probes is preferably carried out by incorporating labeled molecules into the polynucleotides by primer extension, or alternatively by addition to the 5′ or 3′ ends. Examples of nonradioactive labeling of nucleic acid fragments are described in particular in French patent No. 78 109 75 or in the articles by Urdea et al. (98) or Sanchez-Pescador et al. (99).

Preferably, the nucleotide probes and primers according to the invention may have structural characteristics of the type to allow amplification of the signal, such as the probes described by Urdea et al. (100) or alternatively in European patent No. EP-0,225,807 (CHIRON).

The oligonucleotide probes according to the invention may be used in particular in Southern-type hybridizations with the genomic DNA or alternatively in hybridizations with the corresponding messenger RNA when the expression of the corresponding transcript is sought in a sample.

The probes and primers according to the invention may also be used for the detection of products of PCR amplification or alternatively for the detection of mismatches.

Nucleotide probes or primers according to the invention may be immobilized on a solid support. Such solid supports are well known to persons skilled in the art and comprise surfaces of wells of microtiter plates, polystyrene beds, magnetic beds, nitrocellulose bands or microparticles such as latex particles.

Methods for DETECTING Nucleic Acids Encoding RGS18 Polypeptides and RGS18 Polypeptides

The invention also relates to means for the detection of RGS18 nucleic acids, protein, and RGS18 domain comprising polypeptides.

A preferred embodiment of the present invention relates to a method of amplifying a nucleic acid according to the invention, and more particularly a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence; or a nucleic acid fragment or variant thereof contained in a sample, wherein said method comprising the steps of:

a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and

b) detecting the amplified nucleic acids.

The present invention also relates to a method of detecting the presence of a nucleic acid in a sample, wherein the nucleic acid comprises a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence; or a nucleic acid fragment or variant thereof, said method comprising the steps of:

1) bringing one or more nucleotide probes according to the invention into contact with the sample to be tested;

2) detecting the complex which may have formed between the probe(s) and the nucleic acid present in the sample.

According to a specific embodiment of the method of detection according to the invention, the oligonucleotide probes and primers are immobilized on a support.

According to another aspect, the oligonucleotide probes and primers comprise a detectable marker.

The invention relates, in addition, to a box or kit for detecting the presence of a nucleic acid according to the invention in a sample, said box or kit comprising:

a) one or more nucleotide probe(s) or primer(s) as described above;

b) where appropriate, the reagents necessary for the hybridization reaction.

According to a first aspect, the detection box or kit is characterized in that the probe(s) or primer(s) are immobilized on a support.

According to a second aspect, the detection box or kit is characterized in that the oligonucleotide probes comprise a detectable marker.

According to a specific embodiment of the detection kit described above, such a kit will comprise a plurality of oligonucleotide probes and/or primers in accordance with the invention which may be used to detect a target nucleic acid of interest or alternatively to detect mutations in the coding regions or the non-coding regions of the nucleic acids according to the invention.

Another subject of the invention is a box or kit for amplifying all or part of a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence, wherein said box or kit comprises:

1) a pair of nucleotide primers in accordance with the invention, whose hybridization position is located respectively on the 5′ side and 3′ side of the target nucleic acid whose amplification is sought; and optionally,

2) reagents necessary for an amplification reaction.

Such an amplification box or kit will preferably comprise at least one pair of nucleotide primers as described above.

The invention also relates to a box or kit for detecting the presence of a nucleic acid according to the invention in a sample, said box or kit comprising:

a) one or more nucleotide probes according to the invention;

b) where appropriate, reagents necessary for a hybridization reaction.

According to a first aspect, the detection box or kit is characterized in that the nucleotide probe(s) and primer(s) are immobilized on a support.

According to a second aspect, the detection box or kit is characterized in that the nucleotide probe(s) and primer(s) comprise a detectable marker.

According to a specific embodiment of the detection kit described above, such a kit will comprise a plurality of oligonucleotide probes and/or primers in accordance with the invention that may be used to detect target nucleic acids of interest.

According to preferred embodiment of the invention, the target nucleic acid comprises a polynucleotide sequence of a) any one of SEQ ID NOs: 0.11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

Alternatively, the target nucleic acid is a nucleic acid fragment or variant of a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

According to a preferred embodiment, two primers according to the invention comprise all or part of SEQ ID NOs: 9 and 10, making it possible to amplify the region of nucleotides 163-870 of SEQ ID NO: 19, or a nucleic acid having a complementary polynucleotide sequence.

According to a preferred embodiment, two primers according to the invention comprise all or part of SEQ ID NOs: 14 and 15, making it possible to amplify the region of nucleotides 510-839 of SEQ ID NO: 19, or a nucleic acid having a complementary polynucleotide sequence.

According to a preferred embodiment, two primers according to the invention comprise all or part of SEQ ID NOs: 14 and 16, making it possible to amplify the region of nucleotides 510-885 of SEQ ID NO: 19, or a nucleic acid having a complementary polynucleotide sequence.

According to a preferred embodiment, two primers according to the invention comprise all or part of SEQ ID NOs: 14 and 17, making it possible to amplify the region of nucleotides 510-923 of SEQ ID NO: 19, or a nucleic acid having a complementary polynucleotide sequence.

According to another preferred embodiment, a primer according to the invention comprises, generally, all or part of any one of SEQ ID NOs: 9, 10, 14, 15, 16, 17, 30, 31, 32, 33, 34, 35, or 36, or a complementary sequence.

Thus, the probes according to the invention, immobilized on a support, may be ordered into matrices such as “DNA chips”. Such ordered matrices have in particular been described in U.S. Pat. No. 5,143,854, in published PCT applications WO 90/15070 and WO 92/10092.

Support matrices on which oligonucleotide probes have been immobilized at a high density are for example described in U.S. Pat. No. 5,412,087 and in published PCT application WO 95/11995.

The nucleotide primers according to the invention may be used to amplify any one of the nucleic acids according to the invention, or a complementary polynucleotide sequence. Alternatively, the nucleotide primers according to the invention may be used to amplify a nucleic acid fragment or variant of a nucleic acid according to the invention, or a complementary polynucleotide sequence.

Another subject of the invention relates to a method of amplifying a nucleic acid according to the invention, or a complementary polynucleotide sequence, contained in a sample, said method comprising the steps of:

a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and

b) detecting the amplified nucleic acids.

To carry out the amplification method as defined above, use will be preferably made of any of the nucleotide primers described above.

The subject of the invention is, in addition, a box or kit for amplifying all or part of a nucleic acid according to the invention, or a complementary polynucleotide sequence, said box or kit comprising:

a) a pair of nucleotide primers in accordance with the invention, whose hybridization position is located respectively on the 5′ side and 3′ side of the target nucleic acid whose amplification is sought; and optionally,

b) reagents necessary for the amplification reaction.

Such an amplification box or kit will preferably comprise at least one pair of nucleotide primers as described above.

The invention also relates to a box or kit for detecting the presence of a nucleic acid according to the invention in a sample, said box or kit comprising:

a) one or more nucleotide probes according to the invention;

b) where appropriate, reagents necessary for a hybridization reaction.

According to a first aspect, the detection box or kit is characterized in that the nucleotide probe(s) and primer(s) are immobilized on a support.

According to a second aspect, the detection box or kit is characterized in that the nucleotide probe(s) and primer(s) comprise a detectable marker.

According to a specific embodiment of the detection kit described above, such a kit will comprise a plurality of oligonucleotide probes and/or primers in accordance with the invention which may be used to detect target nucleic acids of interest or alternatively to detect mutations in the coding regions or the non-coding regions of the nucleic acids according to the invention.

According to preferred embodiment of the invention, the target nucleic acid comprises a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

Alternatively, the target nucleic acid is a nucleic acid fragment or variant of a nucleic acid comprising a polynucleotide sequence of a) any one of SEQ ID NOs: 11, 18, or 19, or of a complementary polynucleotide sequence, b) nucleotides 1-169 of SEQ ID NO: 1, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

According to a preferred embodiment, a primer according to the invention comprises, generally, all or part of any one of SEQ ID NOs: 9, 10, 14, 15, 16, 17, 30, 31, 32, 33, 34, 35, or 36, or a complementary sequence.

Recombinant Vectors

The invention also relates to a recombinant vector comprising a nucleic acid according to the invention. “Vector” for the purposes of the present invention will be understood to mean a circular or linear DNA or RNA molecule that is either in single-stranded or double-stranded form.

Preferably, such a recombinant vector will comprise a nucleic acid selected from the group consisting of

a) a nucleic acid comprising a polynucleotide sequence of 1) any one of SEQ ID NOs: 11, 18, or 19, or of a complementary polynucleotide sequence, 2) nucleotides 1-169 of SEQ ID NO: 11, or of a complementary polynucleotide sequence, 3) nucleotides 1-658 of SEQ ID NO: 19, or of a complementary polynucleotide sequence, 4) nucleotides 163-870 of SEQ ID NO: 19, or of a complementary polynucleotide sequence, 5) nucleotides 163-658 of SEQ ID NO: 19, or of a complementary polynucleotide-sequence, 6) nucleotides 418-768 of SEQ ID NO: 19, or of a complementary polynucleotide sequence, or 7) nucleotides 418-658 of SEQ ID NO: 19, or of a complementary polynucleotide sequence,

b) a nucleic acid comprising a polynucleotide sequence as depicted in either one of SEQ ID NOs: 18 or 19, or of a complementary polynucleotide sequence,

c) a nucleic acid having at least eight consecutive nucleotides of a nucleic acid comprising a polynucleotide sequence of 1) nucleotides 1-169 of SEQ ID NO: 11, or of a complementary polynucleotide sequence, 2) nucleotides 1-658 of SEQ ID NO: 19, or of a complementary polynucleotide sequence, 3) nucleotides 163-658 of SEQ ID NO: 19, or of a complementary polynucleotide sequence, or 4) nucleotides 418-658 of SEQ ID NO: 19, or of a complementary polynucleotide sequence,

e) a nucleic acid having at least 80% nucleotide identity with a nucleic acid comprising a polynucleotide sequence of 1) any one of SEQ ID NOs: 11, 18, or 19, or of a complementary polynucleotide sequence, 2) nucleotides 1-169 of SEQ ID NO: 11, or of a complementary polynucleotide sequence, 3) nucleotides 1-658 of SEQ ID NO: 19, or of a complementary polynucleotide sequence, 4) nucleotides 163-870 of SEQ ID NO: 19, or of a complementary polynucleotide sequence, 5) nucleotides 163-658 of SEQ ID NO: 19, or of a complementary polynucleotide sequence, 6) nucleotides 418-768 of SEQ ID NO: 19, or of a complementary polynucleotide sequence, or 7) nucleotides 418-658 of SEQ ID NO: 19, or of a complementary polynucleotide sequence,

f) a nucleic acid having 85%, 90%, 95%, or 98% nucleotide identity with a nucleic acid comprising a polynucleotide sequence of 1) any one of SEQ ID NOs: 11, 18, or 19, or of a complementary polynucleotide sequence, 2) nucleotides 1-169 of SEQ ID NO: 11, or of a complementary polynucleotide sequence, 3) nucleotides 1-658 of SEQ ID NO: 19, or of a complementary polynucleotide sequence, 4) nucleotides 163-870 of SEQ ID NO: 19, or of a complementary polynucleotide sequence, 5) nucleotides 163-658 of SEQ ID NO: 19, or of a complementary polynucleotide sequence, 6) nucleotides 418-768 of SEQ ID NO: 19, or of a complementary polynucleotide sequence, or 7) nucleotides 418-658 of SEQ ID NO: 19, or of a complementary polynucleotide sequence,

g) a nucleic acid hybridizing, under high stringency hybridization conditions, with a nucleic acid comprising a polynucleotide sequence of 1) any one of SEQ ID NOs: 11, 18, or 19, or of a complementary polynucleotide sequence, 2) nucleotides 1-169 of SEQ ID NO: 11, or of a complementary polynucleotide sequence, 3) nucleotides 1-658 of SEQ ID NO: 19, or of a complementary polynucleotide sequence, 4) nucleotides 163-870 of SEQ ID NO: 19, or of a complementary polynucleotide sequence, 5) nucleotides 163-658 of SEQ ID NO: 19, or of a complementary polynucleotide sequence, 6) nucleotides 418-768 of SEQ ID NO: 19, or of a complementary polynucleotide sequence, or 7) nucleotides 418-658 of SEQ ID NO: 19, or of a complementary polynucleotide sequence,

h) a nucleic acid encoding a polypeptide comprising an amino acid sequence of SEQ ID NO: 20, and

i) a nucleic acid encoding a polypeptide comprising 1) amino acids 1-58 of SEQ ID NO: 12, 2) amino acids 1-166 of SEQ ID NO: 20, 3) amino acids 86-202 of SEQ ID NO: 20, or 4) amino acids 86-166 of SEQ ID NO: 20.

According to a first embodiment, a recombinant vector according to the invention is used to amplify a nucleic acid inserted therein, following transformation or transfection of a desired cellular host.

According to a second embodiment, a recombinant vector according to the invention corresponds to an expression vector comprising, in addition to a nucleic acid in accordance with the invention, a regulatory signal or nucleotide sequence that directs or controls transcription and/or translation of the nucleic acid and its encoded mRNA.

According to a preferred embodiment, a recombinant vector according to the invention will comprise in particular the following components:

(1) an element or signal for regulating the expression of the nucleic acid to be inserted, such as a promoter and/or enhancer sequence;

(2) a nucleotide coding region comprised within the nucleic acid in accordance with the invention to be inserted into such a vector, said coding region being placed in phase with the regulatory element or signal described in (1); and (3) an appropriate nucleic acid for initiation and termination of transcription of the nucleotide coding region of the nucleic acid described in (2).

In addition, the recombinant vectors according to the invention may include one or more origins for replication in the cellular hosts in which their amplification or their expression is sought, markers or selectable markers.

By way of example, the bacterial promoters may be the LacI or LacZ promoters, the T3 or T7 bacteriophage RNA polymerase promoters, the lambda phage PR or PL promoters.

The promoters for eukaryotic cells will comprise the herpes simplex virus (HSV) virus thymidine kinase promoter or alternatively the mouse metallothionein-L promoter.

Generally, for the choice of a suitable promoter, persons skilled in the art can preferably refer to the book by Sambrook et al. (40) cited above or to the techniques described by Fuller et al. (101).

When the expression of the genomic sequence of the RGS18 gene will be sought, use will preferably be made of the vectors capable of containing large insertion sequences. In a particular embodiment, bacteriophage vectors such as the P1 bacteriophage vectors such as the vector p158 or the vector p158/neo8 described by Sternberg (102, 103) will be preferably used.

The preferred bacterial vectors according to the invention are for example the vectors pBR322 (ATCC37017) or alternatively vectors such as pAA223-3 (Pharmacia, Uppsala, Sweden), and pGEM1 (Promega Biotech, Madison, Wis., UNITED STATES).

There may also be cited other commercially available vectors such as the vectors pQE70, pQE60, pQE9 (Qiagen), psiX174, pBluescript SA, pNH8A, pNH16A, pNH18A, pNH46A, pWLNEO, pSV2CAT, pOG44, pXT1, pSG (Stratagene).

They may also be vectors of the baculovirus type such as the vector pVL1392/1393 (Pharmingen) used to transfect cells of the Sf9 line (ATCC No. CRL 1711) derived from Spodoptera frugiperda.

The present invention also relates to a defective recombinant virus comprising a nucleic acid encoding an RGS18 polypeptide. In a preferred embodiment, the defective recombinant virus comprises a cDNA molecule that encodes an RGS18 polypeptide. In another preferred embodiment of the invention, the defective recombinant virus comprises a gDNA molecule that encodes an RGS18 polypeptide. Preferably, the encoded RGS18 polypeptide comprises amino acids 86-166 of SEQ ID NO: 20. More preferably, the encoded RGS18 polypeptide comprises amino acids 86-202 of SEQ ID NO: 20. Even more preferably, the encoded RGS18 polypeptide comprises an amino acid sequence of SEQ ID NO: 20.

In another preferred embodiment, the invention relates to a defective recombinant virus comprising a nucleic acid encoding an RGS18 protein under the control of a promoter chosen from Rous sarcoma virus-long terminal repeat (RSV-LTR) or the cytomegalovirus (CMV) early promoter.

They may also be adenoviral vectors such as the human adenovirus of type 2 or 5.

A recombinant vector according to the invention may also be a retroviral vector or an adeno-associated vector (AAV). Such adeno-associated vectors are for example described by references 104-106.

To allow the expression of a polynucleotide according to the invention, the latter must be introduced into a host cell. The introduction of a polynucleotide according to the invention into a host cell may be carried out in vitro, according to the techniques well known to persons skilled in the art for transforming or transfecting cells, either in primer culture, or in the form of cell lines. It is also possible to carry out the introduction of a polynucleotide according to the invention in vivo or ex vivo, for the prevention or treatment of diseases linked to a deficiency in the reverse transport of cholesterol.

To introduce a polynucleotide or vector of the invention into a host cell, a person skilled in the art can preferably refer to various techniques, such as the calcium phosphate precipitation technique (107, 108), DEAE Dextran (109), electroporation (110, 111), direct microinjection (112), liposomes charged with DNA (113, 114).

Once the polynucleotide has been introduced into the host cell, it may be stably integrated into the genome of the cell. The intregration may be achieved at a precise site of the genome, by homologous recombination, or it may be randomly integrated. In some embodiments, the polynucleotide may be stably maintained in the host cell in the form of an episome fragment, the episome comprising sequences allowing the retention and the replication of the latter, either independently, or in a synchronized manner with the cell cycle.

According to a specific embodiment, a method of introducing a polynucleotide according to the invention into a host cell, in particular a host cell obtained from a mammal, in vivo, comprises a step during which a preparation comprising a pharmaceutically compatible vector and a “naked” polynucleotide according to the invention, placed under the control of appropriate regulatory sequences, is introduced by local injection at the level of the chosen tissue, for example a smooth muscle tissue, the “naked” polynucleotide being absorbed by the cells of this tissue.

Compositions for use in vitro and in vivo comprising “naked” polynucleotides are for example described in PCT Application No. WO 95/11307 (Institut Pasteur, Inserm, University of Ottawa) as well as in the articles by Tacson et al. (115) and Huygen et al. (58).

According to a specific embodiment of the invention, a composition is provided for the in vivo production of the RSG18 protein. This composition comprises a polynucleotide encoding the RGS18 polypeptide placed under the control of appropriate regulatory sequences, in solution in a physiologically acceptable vehicle or excipient.

Consequently, the invention also relates to a pharmaceutical composition intended for the prevention of or treatment of a patient or subject affected by a disorder or condition associated with platelet activation dysfunction, comprising a nucleic acid encoding the RGS18 protein, in combination with one or more physiologically compatible excipients.

Preferably, such a composition will comprise a nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, org) nucleotides 418-658 of SEQ ID NO: 19, or of a complementary polynucleotide sequence, wherein the nucleic acid is placed under the control of an appropriate regulatory element or signal.

The subject of the invention is, in addition, a pharmaceutical composition intended for the prevention of or treatment of a patient or a subject affected by a disorder or condition associated with platelet activation dysfunction, comprising a recombinant vector according to the invention, in combination with one or more physiologically compatible excipients.

The quantity of vector that is injected into the host organism chosen varies according to the site of the injection. As a guide, there may be injected between about 0.1 and about 100 μg of polynucleotide encoding a RGS18 polypeptide into the body of an animal, preferably into a patient likely to develop a disorder or condition associated with platelet activation dysfunction or a patient who has already developed the disorder or condition.

The invention also relates to the use of a nucleic acid according to the invention, encoding the RGS18 protein, for the manufacture of a medicament intended for the prevention of a disorder or condition associated with platelet activation dysfunction in various forms or more particularly for the treatment of subjects affected by a disorder or condition associated with platelet activation dysfunction.

The invention also relates to the use of a recombinant vector according to the invention, comprising a nucleic acid encoding the RGS18 protein, for the manufacture of a medicament intended for the prevention of, or more particularly for the treatment of subjects affected by a disorder or condition associated with platelet activation dysfunction.

The subject of the invention is therefore also a recombinant vector comprising a nucleic acid according to the invention that encodes an RGS18 protein or polypeptide involved in platelet activation.

The invention also relates to the use of such a recombinant vector for the preparation of a pharmaceutical composition intended for the treatment and/or for the prevention of a disorder or condition associated with platelet activation dysfunction.

The present invention also relates to the use of cells genetically modified ex vivo with such a recombinant vector according to the invention, or of cells producing a recombinant vector, wherein the cells are implanted in the body, to allow a prolonged and effective expression in vivo of a biologically active RGS18 polypeptide.

The invention also relates to the use of a nucleic acid according to the invention encoding an RGS18 protein for the manufacture of a medicament intended for the prevention or treatment of subjects affected by a disorder or condition associated with platelet activation dysfunction.

The invention also relates to the use of a recombinant vector according to the invention comprising a nucleic acid encoding an RGS18 polypeptide according to the invention for the manufacture of a medicament intended for the prevention of, or more particularly, for the treatment of subjects affected by a disorder or condition associated with platelet activation dysfunction.

The invention also relates to the use of a recombinant host cell according to the invention, comprising a nucleic acid encoding an RGS18 polypeptide according to the invention for the manufacture of a medicament intended for the prevention of, or more particularly, for the treatment of subjects affected by a disorder or condition associated with platelet activation dysfunction.

The present invention also relates to the use of a recombinant vector according to the invention, preferably a defective recombinant virus, for the preparation of a pharmaceutical composition for the treatment and/or prevention of pathologies linked to platelet activation dysfunction.

The invention relates to the use of such a recombinant vector or defective recombinant virus for the preparation of a pharmaceutical composition intended for the treatment and/or for the prevention of a disorder or condition associated with platelet activation dysfunction. Thus, the present invention also relates to a pharmaceutical composition comprising one or more recombinant vectors or defective recombinant viruses according to the invention.

The present invention also relates to a new therapeutic approach for the prevention and/or treatment of pathologies linked to platelet activation dysfunction. It provides an advantageous solution to the disadvantages of the prior art, by demonstrating the possibility of treating the pathologies linked to platelet activation dysfunction by gene therapy, by the transfer and expression in vivo of a nucleic acid encoding an RGS18 polypeptide involved in platelet activation. The invention thus offers a simple means allowing a specific and effective treatment of related pathologies such as, for example, arterial thrombosis, myocardial infarction, coronary artery disease, stroke, cerebrovascular disease, unstable angina, deep vein thrombosis, systemic thromboembolism, as well as its use in invasive cardiac procedures for anti-coagulant purposes.

Gene therapy consists in correcting a deficiency or an abnormality (mutation, aberrant expression and the like) and in bringing about the expression of a protein of therapeutic interest by introducing genetic information into the affected cell or organ. This genetic information may be introduced either ex vivo into a cell extracted from the organ, the modified cell then being reintroduced into the body, or directly in vivo into the appropriate tissue. In this second case, various techniques exist, among which various transfection techniques involving complexes of DNA and DEAE-dextran (116), of DNA and nuclear proteins (117), of DNA and lipids (87), the use of liposomes (118), and the like. More recently, the use of viruses as vectors for the transfer of genes has appeared as a promising alternative to these physical transfection techniques. In this regard, various viruses have been tested for their capacity to infect certain cell populations. In particular, the retroviruses (RSV, HMS, MMS, and the like), the HSV virus, the adeno-associated viruses and the adenoviruses.

The present invention therefore also relates to a new therapeutic approach for the treatment of a disorder or condition associated with platelet activation dysfunction, comprising in transferring and in expressing in vivo genes encoding RGS18. Specifically, the present invention provides a new therapeutic approach for the treatment and/or prevention of a disorder or condition associated with platelet activation dysfunction, such as arterial thrombosis, myocardial infarction, coronary artery disease, stroke, cerebrovascular disease, unstable angina, deep vein thrombosis, systemic thromboembolism, as well as its use in invasive cardiac procedures for anti-coagulant purposes. In a particularly preferred manner, the applicant has now found that it is possible to construct recombinant vectors comprising a nucleic acid encoding an RGS18 polypeptide involved in the platelet activation, to administer these recombinant vectors in vivo, and that this administration allows a stable and effective expression of a biologically active RGS18 polypeptide in vivo, with no cytopathological effect.

The present invention also results from the demonstration that adenoviruses constitute particularly efficient vectors for the transfer and the expression of the RGS18 nucleic acids of the invention. In particular, the present invention shows that the use of recombinant adenoviruses as vectors makes it possible to obtain sufficiently high levels of expression of this gene to produce the desired therapeutic effect. Other viral vectors such as retroviruses or adeno-associated viruses (AAV) allowing a stable expression of the gene are also claimed.

The present invention thus offers a new approach for the treatment and prevention of a disorder or condition associated with platelet activation dysfunction.

The subject of the invention is therefore also a defective recombinant virus comprising a nucleic acid according to the invention that encodes an RGS18 protein or polypeptide involved in platelet activation.

The invention also relates to the use of such a defective recombinant virus for the preparation of a pharmaceutical composition intended for the treatment and/or for the prevention of a disorder or condition associated with platelet activation dysfunction, such as arterial thrombosis, myocardial infarction, coronary artery disease, stroke, cerebrovascular disease, unstable angina, deep vein thrombosis, systemic thromboembolism, as well as its use in invasive cardiac procedures for anti-coagulant purposes.

The present invention also relates to the use of cells genetically modified ex vivo with such a defective recombinant virus according to the invention, or of cells producing a defective recombinant virus, wherein the cells are implanted in the body, to allow a prolonged and effective expression in vivo of a biologically active RGS18 polypeptide.

The present invention shows that it is possible to incorporate a nucleic acid sequence encoding RGS18 into a viral vector, and that these vectors make it possible to effectively express a biologically active, mature form. More particularly, the invention shows that the in vivo expression of RGS18 may be obtained by direct administration of an adenovirus or by implantation of a producing cell or of a cell genetically modified by an adenovirus or by a retrovirus incorporating such a nucleic acid.

The present invention is particularly advantageous because it makes it possible to induce a controlled expression, and with no harmful effect, of RGS18 in organs which are not normally involved in the expression of this protein. In particular, a significant release of the RGS18 protein is obtained by implantation of cells producing vectors of the invention, or infected ex vivo with vectors of the invention.

The nucleic sequence used in the context of the present invention may be a cDNA, a genomic DNA (gDNA), an RNA (in the case of retroviruses) or a hybrid construct consisting, for example, of a cDNA into which one or more introns (gDNA) would be inserted. It may also involve synthetic or semisynthetic sequences. In a particularly advantageous manner, a cDNA or a gDNA is used. In particular, the use of a gDNA allows a better expression in human cells. To allow their incorporation into a viral vector according to the invention, these sequences are preferably modified, for example by site-directed mutagenesis, in particular for the insertion of appropriate restriction sites.

In the context of the present invention, the use of a nucleic acid encoding a human RGS18 protein is preferred. Moreover, it is also possible to use a construct encoding a derivative of these RGS18 proteins. A derivative of these RGS18 proteins comprises, for example, any sequence obtained by mutation, deletion and/or addition relative to the native sequence, and encoding a product retaining biological activity. These modifications may be made by techniques known to a person skilled in the art (see general molecular biological techniques below). The biological activity of the derivatives thus obtained can then be easily determined, as indicated in particular in the examples. The derivatives for the purposes of the invention may also be obtained by hybridization from nucleic acid libraries, using as probe the native sequence or a fragment thereof.

These derivatives are in particular molecules having a higher affinity for their binding sites, molecules exhibiting greater resistance to proteases, molecules having a higher therapeutic efficacy or fewer side effects, or optionally new biological properties. The derivatives also include the modified DNA sequences allowing improved expression in vivo.

Thus, the present invention relates to a defective recombinant virus comprising a nucleic acid that encodes an RGS18 polypeptide. In a first embodiment, the present invention relates to a defective recombinant virus comprising a cDNA that encodes an RGS18 polypeptide. In another preferred embodiment of the invention, a defective recombinant virus comprises a genomic DNA (gDNA) that encodes an RGS18 polypeptide. Preferably, the encoded RGS18 polypeptide comprises amino acids a) 1-58 of SEQ ID NO: 12, b) 1-166 of SEQ ID NO: 20, c) 86-202 of SEQ ID NO: 20, or d) 86-166 of SEQ ID NO: 20. More preferably, the encoded RGS18 polypeptide an amino acid sequence of SEQ ID NO: 20.

The vectors of the invention may be prepared from various types of viruses. Preferably, vectors derived from adenoviruses, adeno-associated viruses (AAV), herpesviruses (HSV) or retroviruses are used. It is preferable to use an adenovirus, for direct administration or for the ex vivo modification of cells intended to be implanted, or a retrovirus, for the implantation of producing cells.

The viruses according to the invention are defective; that is to say that they are incapable of autonomously replicating in the target cell. Generally, the genome of the defective viruses used in the context of the present invention therefore lacks at least the sequences necessary for the replication of said virus in the infected cell. These regions may be either eliminated (completely or partially), or made nonfunctional, or substituted with other sequences and in particular with the nucleic acid sequence encoding the RGS18 polypeptide. Preferably, the defective virus retains, nevertheless, the sequences of its genome that are necessary for the encapsidation of the viral particles.

As regards more particularly adenoviruses, various serotypes, whose structure and properties vary somewhat, have been characterized. Among these serotypes, human adenoviruses of type 2 or 5 (Ad 2 or Ad 5) or adenoviruses of animal origin (see Application WO 94/26914) are preferably used in the context of the present invention. Among the adenoviruses of animal origin that can be used in the context of the present invention, there may be mentioned adenoviruses of canine, bovine, murine (example: Mav1, 119), ovine, porcine, avian or simian (example: SAV) origin. Preferably, the adenovirus of animal origin is a canine adenovirus, more preferably a CAV2 adenovirus [Manhattan or A26/61 strain (ATCC VR-800) for example]. Preferably, adenoviruses of human or canine or mixed origin are used in the context of the invention. Preferably, the defective adenoviruses of the invention comprise the ITRs, a sequence allowing the encapsidation and the sequence encoding the RGS18 polypeptide. Preferably, in the genome of the adenoviruses of the invention, the E1 region at least is made nonfunctional. Still more preferably, in the genome of the adenoviruses of the invention, the E1 gene and at least one of the E2, E4 and L1-L5 genes are nonfunctional. The viral gene considered may be made nonfunctional by any technique known to a person skilled in the art, and in particular by total suppression, by substitution, by partial deletion or by addition of one or more bases in the gene(s) considered. Such modifications may be obtained in vitro (on the isolated DNA) or in situ, for example, by means of genetic engineering techniques, or by treatment by means of mutagenic agents. Other regions may also be modified, and in particular the E3 (WO95/02697), E2 (WO94/28938), E4 (WO94/28152, WO94/12649, WO95/02697) and L5 (WO95/02697) region. According to a preferred embodiment, the adenovirus according to the invention comprises a deletion in the E1 and E4 regions and the sequence encoding RGS18 is inserted at the level of the inactivated E1 region. According to another preferred embodiment, it comprises a deletion in the E1 region at the level of which the E4 region and the sequence encoding RGS18 (French Patent Application FR94 13355) are inserted.

The defective recombinant adenoviruses according to the invention may be prepared by any technique known to persons skilled in the art (EP 185 573; and 120, 121). In particular, they may be prepared by homologous recombination between an adenovirus and a plasmid carrying, inter alia, the nucleic acid encoding the RGS18 protein. The homologous recombination occurs after co-transfection of said adenoviruses and plasmid into an appropriate cell line. The cell line used must preferably (i) be transformable by said elements, and (ii), contain the sequences capable of complementing the part of the defective adenovirus genome, preferably in integrated form in order to avoid the risks of recombination. By way of example of a line, there may be mentioned the human embryonic kidney line 293 (122), which contains in particular, integrated into its genome, the left part of the genome of an Ad5 adenovirus (12%) or lines capable of complementing the E1 and E4 functions as described in particular in Applications No. WO 94/26914 and WO95/02697.

Next, the adenoviruses that have multiplied are recovered and purified according to conventional molecular biological techniques, as illustrated in the examples.

As regards the adeno-associated viruses (AAV), they are DNA viruses of a relatively small size, which integrate into the genome of the cells that they infect, in a stable and site-specific manner. They are capable of infecting a broad spectrum of cells, without inducing any effect on cellular growth, morphology or differentiation. Moreover, they do not appear to be involved in pathologies in humans. The genome of AAVs has been cloned, sequenced and characterized. It comprises about 4700 bases, and contains at each end an inverted repeat region (ITR) of about 145 bases, serving as replication origin for the virus. The remainder of the genome is divided into 2 essential regions carrying the encapsidation functions: the left hand part of the genome, which contains the rep gene, involved in the viral replication and the expression of the viral genes; the right hand part of the genome, which contains the cap gene encoding the virus capsid proteins.

The use of vectors derived from AAVs for the transfer of genes in vitro and in vivo has been described in the literature (see in particular WO 91/18088; WO 93/09239; U.S. Pat. No. 4,797,368, U.S. Pat. No. 5,139,941, EP 488 528). These applications describe various constructs derived from AAVs, in which the rep and/or cap genes are deleted and replaced by a gene of interest, and their use for transferring in vitro (on cells in culture) or in vivo (directly into an organism) said gene of interest. However, none of these documents either describes or suggests the use of a recombinant AAV for the transfer and expression in vivo or ex vivo of an RGS18 protein, or the advantages of such a transfer. The defective recombinant AAVs according to the invention may be prepared by co-transfection, into, a cell line infected with a human helper virus (for example an adenovirus), of a plasmid containing the sequence encoding the RGS18 protein bordered by two AAV inverted repeat regions (ITR), and of a plasmid carrying the AAV encapsidation genes (rep and cap genes). The recombinant AAVs produced are then purified by conventional techniques.

As regards the herpesviruses and the retroviruses, the construction of recombinant vectors has been widely described in the literature: see in particular WO 94/21807, WO 92/05263, EP 453242, EP 178220, and 123-125, and the like.

In particular, the retroviruses are integrating viruses, infecting dividing cells. The genome of the retroviruses essentially comprises two long terminal repeats (LTRs), an encapsidation sequence and three coding regions (gag, pol and env). In the recombinant vectors derived from retroviruses, the gag, pol and env genes are generally deleted, completely or partially, and replaced with a heterologous nucleic acid sequence of interest. These vectors may be produced from various types of retroviruses such as in particular MoMuLV (“murine moloney leukemia virus”; also called MoMLV), MSV (“murine moloney sarcoma virus”), HaSV (“harvey sarcoma virus”); SNV (“spleen necrosis virus”); RSV (“rous sarcoma virus”) or Friend's virus.

To construct recombinant retroviruses containing a sequence encoding the RGS18 protein according to the invention, a plasmid containing in particular the LTRs, the encapsidation sequence and said coding sequence is generally constructed, and then used to transfect a so-called encapsidation cell line, capable of providing in trans the retroviral functions deficient in the plasmid. Generally, the encapsidation lines are therefore capable of expressing the gag, pol and env genes. Such encapsidation lines have been described in the prior art, and in particular the PA317 line (U.S. Pat. No. 4,861,719), the PsiCRIP line (WO 90/02806) and the GP+envAm-12 line (WO 89/07150). Moreover, the recombinant retroviruses may contain modifications at the level of the LTRs in order to suppress the transcriptional activity, as well as extended encapsidation sequences, containing a portion of the gag gene (126). The recombinant retroviruses produced are then purified by conventional techniques.

To carry out the present invention, it is preferable to use a defective recombinant adenovirus. The particularly advantageous properties of adenoviruses are preferred for the in vivo expression of a protein having a cholesterol transport activity. The adenoviral vectors according to the invention are particularly preferred for a direct administration in vivo of a purified suspension, or for the ex vivo transformation of cells, in particular autologous cells, in view of their implantation. Furthermore, the adenoviral vectors according to the invention exhibit, in addition, considerable advantages, such as in particular their very high infection efficiency, which makes it possible to carry out infections using small volumes of viral suspension.

According to another particularly preferred embodiment of the invention, a line producing retroviral vectors containing the sequence encoding the RGS18 protein is used for implantation in vivo. The lines that can be used to this end are in particular the PA317 (U.S. Pat. No. 4,861,719), PsiCrip (WO 90/02806) and GP+envAm-12 (U.S. Pat. No. 5,278,056) cells modified so as to allow the production of a retrovirus containing a nucleic sequence encoding an RGS18 protein according to the invention.

Preferably, in the vectors of the invention, the nucleic acid encoding the RGS18 protein is placed under the control of signals allowing its expression in the infected cells. These may be expression signals that are homologous or heterologous, that is to say signals different from those that are naturally responsible for the expression of the RGS18 protein. They may also be in particular sequences responsible for the expression of other proteins, or synthetic sequences. In particular, they may be sequences of eukaryotic or viral genes or derived sequences, stimulating or repressing the transcription of a gene in a specific manner or otherwise and in an inducible manner or otherwise. By way of example, they may be promoter sequences derived from the genome of the cell which it is desired to infect, or from the genome of a virus, and in particular the promoters of the E1A or major late promoter (MLP) genes of adenoviruses, the cytomegalovirus (CMV) promoter, the RSV-LTR and the like. Among the eukaryotic promoters, there may also be mentioned the ubiquitous promoters (HRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), tissue-specific promoters (pyruvate kinase, villin, promoter of the fatty acid binding intestinal protein, promoter of the smooth muscle cell α-actin, promoters specific for the liver; Apo AI, Apo AII, human albumin and the like) or promoters corresponding to a stimulus (steroid hormone receptor, retinoic acid receptor and the like). In addition, these expression sequences may be modified by addition of enhancer or regulatory sequences and the like. Moreover, when the inserted gene does not contain expression sequences, it may be inserted into the genome of the defective virus downstream of such a sequence.

In a specific embodiment, the invention relates to a defective recombinant virus comprising a nucleic acid encoding an RGS18 protein operatively linked or placed under the control of a promoter chosen from RSV-LTR or the CMV early promoter.

As indicated above, the present invention also relates to any use of a virus as described above for the preparation of a pharmaceutical composition for the treatment and/or prevention of a disorder or condition associated with platelet activation dysfunction.

The present invention also relates to a pharmaceutical composition comprising one or more defective recombinant viruses as described above. These pharmaceutical compositions may be formulated for administration by the topical, oral, parenteral, intranasal, intravenous, intramuscular, subcutaneous, intraocular or transdermal route and the like. Preferably, the pharmaceutical compositions of the invention comprise a pharmaceutically acceptable vehicle or physiologically compatible excipient for an injectable formulation, in particular for an intravenous injection, such as for example into the patient's portal vein. These may relate in particular to isotonic sterile solutions or dry, in particular, freeze-dried, compositions, which upon addition depending on the case of sterilized water or physiological saline, allow the preparation of injectable solutions. Direct injection into the patient's portal vein is preferred because it makes it possible to target the infection at the level of the liver and thus to concentrate the therapeutic effect at the level of this organ.

The doses of defective recombinant virus used for the injection may be adjusted as a function of various parameters, and in particular as a function of the viral vector, of the mode of administration used, of the relevant pathology or of the desired duration of treatment. In general, the recombinant adenoviruses according to the invention are formulated and administered in the form of doses of between 10⁴ and 10¹⁴ pfu/ml, and preferably 10⁶ to 10¹⁰ pfu/ml. The term “pfu” (plaque forming unit) corresponds to the infectivity of a virus solution, and is determined by infecting an appropriate cell culture and measuring, generally after 48 hours, the number of plaques that result from infected cell lysis. The techniques for determining the pfu titer of a viral solution are well documented in the literature.

As regards retroviruses, the compositions according to the invention may directly contain the producing cells, with a view to their implantation.

In this regard, another subject of the invention relates to any isolated mammalian cell infected with one or more defective recombinant viruses according to the invention. More particularly, the invention relates to any population of human cells infected with such viruses. These may be in particular cells of blood origin (totipotent stem cells or precursors), fibroblasts, myoblasts, hepatocytes, keratinocytes, smooth muscle and endothelial cells, glial cells and the like.

The cells according to the invention may be derived from primary cultures. These may be collected by any technique known to persons skilled in the art and then cultured under conditions allowing their proliferation. As regards more particularly fibroblasts, these may be easily obtained from biopsies, for example according to the technique described by Ham (1980). These cells may be used directly for infection with the viruses, or stored, for example by freezing, for the establishment of autologous libraries, in view of a subsequent use. The cells according to the invention may be secondary cultures, obtained for example from pre-established libraries (see for example EP 228458, EP 289034, EP 400047, EP 456640).

The cells in culture are then infected with a recombinant virus according to the invention, in order to confer on them the capacity to produce a biologically active RGS18 protein. The infection is carried out in vitro according to techniques known to persons skilled in the art. In particular, depending on the type of cells used and the desired number of copies of virus per cell, persons skilled in the art can adjust the multiplicity of infection and optionally the number of infectious cycles produced. It is clearly understood that these steps must be carried out under appropriate conditions of sterility when the cells are intended for administration in vivo. The doses of recombinant virus used for the infection of the cells may be adjusted by persons skilled in the art according to the desired aim. The conditions described above for the administration in vivo may be applied to the infection in vitro. For the infection with a retrovirus, it is also possible to co-culture a cell to be infected with a cell producing the recombinant retrovirus according to the invention. This makes it possible to eliminate purification of the retrovirus.

Another subject of the invention relates to an implant comprising isolated mammalian cells infected with one or more defective recombinant viruses according to the invention or cells producing recombinant viruses, and an extracellular matrix. Preferably, the implants according to the invention comprise 10⁵ to 10¹⁰ cells. More preferably, they comprise 10⁶ to 10⁸ cells.

More particularly, in the implants of the invention, the extracellular matrix comprises a gelling compound and optionally a support allowing the anchorage of the cells.

For the preparation of the implants according to the invention, various types of gelling agents may be used. The gelling agents are used for the inclusion of the cells in a matrix having the constitution of a gel, and for promoting the anchorage of the cells on the support, where appropriate. Various cell adhesion agents can therefore be used as gelling agents, such as in particular collagen, gelatin, glycosaminoglycans, fibronectin, lectins and the like. Preferably, collagen is used in the context of the present invention. This may be collagen of human, bovine or murine origin. More preferably, type I collagen is used.

As indicated above, the compositions according to the invention preferably comprise a support allowing the anchorage of the cells. The term anchorage designates any form of biological and/or chemical and/or physical interaction causing the adhesion and/or the attachment of the cells to the support. Moreover, the cells may either cover the support used, or penetrate inside this support, or both. It is preferable to use in the context of the invention a solid, nontoxic and/or biocompatible support. In particular, it is possible to use polytetrafluoroethylene (PTFE) fibers or a support of biological origin.

The present invention thus offers a very effective means for the treatment or prevention of a disorder or condition associated with platelet activation dysfunction.

In addition, this treatment may be applied to both humans and any animals such as ovines, bovines, domestic animals (dogs, cats and the like), horses, fish and the like.

Recombinant Host Cells

The present invention also relates to the use of cells genetically modified ex vivo with a virus according to the invention, or of cells producing such viruses, implanted in the body, allowing a prolonged and effective expression in vivo of a biologically active RSG18 protein.

The present invention shows that it is possible to incorporate a nucleic acid encoding an RGS18 polypeptide according to the invention into a viral vector, and that these vectors make it possible to effectively express a biologically active, mature polypeptide. More particularly, the invention shows that the in vivo expression of RSG18 may be obtained by direct administration of an adenovirus or by implantation of a producing cell or of a cell genetically modified by an adenovirus or by a retrovirus incorporating such a nucleic acid.

In this regard, another subject of the invention relates to any isolated mammalian cell infected with one or more defective recombinant viruses according to the invention. More particularly, the invention relates to any population of human cells infected with these viruses. These may be in particular cells of blood origin (totipotent stem cells or precursors), fibroblasts, myoblasts, hepatocytes, keratinocytes, smooth muscle and endothelial cells, glial cells and the like.

Another subject of the invention relates to an implant comprising isolated mammalian cells infected with one or more defective recombinant viruses according to the invention or cells producing recombinant viruses, and an extracellular matrix. Preferably, the implants according to the invention comprise 10⁵ to 10¹⁰ cells. More preferably, they comprise 10⁶ to 10⁸ cells.

More particularly, in the implants of the invention, the extracellular matrix comprises a gelling compound and optionally, a support allowing the anchorage of the cells.

The invention also relates to an isolated recombinant host cell comprising a nucleic acid of the invention, and more particularly, a nucleic acid comprising a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The invention also relates to an isolated recombinant host cell comprising a nucleic acid of the invention, and more particularly a nucleic acid comprising a nucleotide sequence as depicted in either SEQ ID NOs: 18 or 19, or of a complementary polynucleotide sequence.

The invention also relates to an isolated recombinant host cell comprising a nucleic acid encoding a polypeptide comprising an amino acid sequence of SEQ ID NO: 20.

The invention also relates to a recombinant host cell comprising a nucleic acid encoding a polypeptide comprising a) amino acids 1-58 of SEQ ID NO: 12, b) amino acids 1-166 of SEQ ID NO: 20, c) amino acids 86-202 of SEQ ID NO: 20, or d) amino acids 86-166 of SEQ ID NO: 20.

According to another aspect, the invention also relates to an isolated recombinant host cell comprising a recombinant vector according to the invention. Therefore, the invention also relates to a recombinant host cell comprising a recombinant vector comprising any of the nucleic acids of the invention.

Specifically, the invention relates to an isolated recombinant host cell comprising a recombinant vector comprising a nucleic acid comprising a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

The invention also relates to an isolated recombinant host cell comprising a recombinant vector comprising a nucleic acid comprising a polynucleotide sequence as depicted in either one of SEQ ID NOs: 18 or 19, or of a complementary polynucleotide sequence.

The invention also relates to an isolated recombinant host cell comprising a recombinant vector comprising a nucleic acid encoding a polypeptide comprising an amino acid sequence of SEQ ID NO:20.

The invention also relates to an isolated recombinant host cell comprising a recombinant vector comprising a nucleic acid encoding a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20.

The preferred host cells according to the invention are for example the following:

a) prokaryotic host cells: strains of Escherichia coli (strain DH5-ax), of Bacillus subtilis, of Salmonella typhimurium, or strains of genera such as Pseudomonas, Streptomyces and Staphylococus;

b) eukaryotic host cells: HeLa cells (ATCC No. CCL2), Cv 1 cells (ATCC No. CCL70), COS cells (ATCC No. CRL 1650), Sf-9 cells (ATCC No. CRL 1711), CHO cells (ATCC No. CCL-61) or 3T3 cells (ATCC No. CRL-6361).

Methods for Producing RGS18 Polypeptides

The invention also relates to a method for the production of a polypeptide comprising an amino acid sequence of either one of SEQ ID NOs: 12 or 20, or of a polypeptide or a variant thereof, wherein the polypeptide or variant comprises amino acids a) 1-58 of SEQ ID NO: 12, b) 1-166 of SEQ ID NO: 20, c) 86-202 of SEQ ID NO: 20, or d) 86-166 of SEQ ID NO: 20, wherein said method comprising the steps of:

a) inserting a nucleic acid encoding said polypeptide into an appropriate vector;

b) culturing, in an appropriate culture medium, a previously transformed host cell or transfecting a host cell with the recombinant vector of step a);

c) recovering the conditioned culture medium or lysing the host cell, for example by sonication or by osmotic shock;

d) separating and purifying said polypeptide from said culture medium or alternatively from the cell lysates obtained in step c); and

e) where appropriate, characterizing the recombinant polypeptide produced.

A specific embodiment of the invention relates to a method for producing a polypeptide comprising an amino acid sequence of amino acids 86-202 of SEQ ID NO: 20.

A polypeptide termed “homologous” to a polypeptide having an amino acid sequence comprising a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20, also forms part of the invention. Such a homologous polypeptide comprises an amino acid sequence possessing one or more substitutions of an amino acid by an equivalent amino acid, relative to a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20, respectively.

The RGS18 polypeptides according to the invention, in particular comprise:

1) a polypeptide comprising an amino acid sequence of either one of SEQ ID NOs: 12 or 20,

2) a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20,

3) a polypeptide fragment or variant of a polypeptide comprising an amino acid sequence of either one of SEQ ID NOs: 12 or 20, wherein the polypeptide fragment or variant comprises amino acids a) 1-58 of SEQ ID NO: 12, b) 1-166 of SEQ ID NO: 20, c) 86-202 of SEQ ID NO: 20, or d) 86-166 of SEQ ID NO: 20, or

4) a polypeptide termed “homologous” to a polypeptide comprising a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

The polypeptides according to the invention may be characterized by binding to an immunoaffinity chromatography column on which the antibodies directed against this polypeptide or against a fragment or a variant thereof have been previously immobilized.

According to another aspect, a recombinant polypeptide according to the invention may be purified by passing it over an appropriate series of chromatography columns, according to methods known to persons skilled in the art and described for example in F. Ausubel et al. (47).

A polypeptide according to the invention may also be prepared by conventional chemical synthesis techniques either in homogeneous solution or in solid phase. By way of illustration, a polypeptide according to the invention may be prepared by the technique either in homogeneous solution described by Houben Weyl (127) or the solid phase synthesis technique described by Merrifield (128, 129).

An “equivalent amino acid” according to the present invention will be understood to mean for example replacement of a residue in the L form by a residue in the D form or the replacement of a glutamic acid (E) by a pyro-glutamic acid according to techniques well known to persons skilled in the art. By way of illustration, the synthesis of peptide containing at least one residue in the D form is described by Koch (130). According to another aspect, two amino acids belonging to the same class, that is to say two uncharged polar, nonpolar, basic or acidic amino acids, are also considered as equivalent amino acids.

Polypeptides comprising at least one nonpeptide bond such as a retro-inverse bond (NHCO), a carba bond (CH₂CH₂) or a ketomethylene bond (CO—CH₂) also form part of the invention.

Preferably, the polypeptides according to the invention comprising one or more additions, deletions, substitutions of at least one amino acid will retain their capacity to be recognized by antibodies directed against the nonmodified polypeptides.

Antibodies

The RGS18 polypeptides according to the invention may be used for the preparation of an antibody, in particular for detecting the production of a normal or altered form of an RGS18 polypeptide in a patient.

Thus, the present invention also relates to antibodies directed against an RGS18 polypeptide. In a specific embodiment, an antibody according to the invention is directed against

1) a polypeptide comprising an amino acid sequence of any one of SEQ ID NOs: 7, 8, 12 or 20,

2) a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20,

3) a polypeptide fragment or variant of a polypeptide comprising an amino acid sequence of either one of SEQ ID NOs: 12 or 20, wherein the polypeptide fragment or variant comprises amino acids a) 1-58 of SEQ ID NO: 12, b) 1-166 of SEQ ID NO: 20, c) 86-202 of SEQ ID NO: 20, or d) 86-166 of SEQ ID NO: 20, or

4) a polypeptide termed “homologous” to a polypeptide comprising a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

The present invention relates to an antibody directed against an RGS18 polypeptide, as produced in the trioma technique or the hybridoma technique described by Kozbor et al. (131).

An antibody directed against a polypeptide termed “homologous” to a polypeptide according to the invention also forms part of the invention. Such an antibody is directed against a homologous polypeptide comprising an amino acid sequence that has one or more substitutions of an amino acid by an equivalent amino acid, relative to a polypeptide according to the invention, wherein the polypeptide according to the invention comprises a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

“Antibody” for the purposes of the present invention will be understood to mean in particular polyclonal or monoclonal antibodies or fragments (for example the F(ab)′₂ and Fab fragments) or any polypeptide comprising a domain of the initial antibody recognizing the target polypeptide or polypeptide fragment according to the invention.

Monoclonal antibodies may be prepared from hybridomas according to the technique described by Kohler and Milstein (132).

According to the invention, a polypeptide produced recombinantly or by chemical synthesis, and fragments or other derivatives or analogs thereof, including fusion proteins, may be used as an immunogen to generate antibodies that recognize a polypeptide according to the invention. Such antibodies include but are not limited to polyclonal, monoclonal, chimeric, single chain, Fab fragments, and an Fab expression library. The anti-RGS18 antibodies of the invention may be cross reactive, e.g., they may recognize an RGS18 polypeptide from different species. Polyclonal antibodies have greater likelihood of cross reactivity. Alternatively, an antibody of the invention may be specific for a single form of RGS18. Preferably, such an antibody is specific for human RGS18.

Various procedures known in the art may be used for the production of polyclonal antibodies to an RGS18 polypeptide or derivative or analog thereof. For the production of antibody, various host animals can be immunized by injection with an RGS18 polypeptide, or a derivative (e.g., fragment or fusion protein) thereof, including but not limited to rabbits, mice, rats, sheep, goats, etc. In one embodiment, the RGS18 polypeptide or fragment thereof can be conjugated to an immunogenic carrier, e.g., bovine serum albumin 03SA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the RGS18 polypeptide, or fragment, analog, or derivative thereof, any technique that provides for the production of antibody molecules by continuous cell lines in culture may be used. These include but are not limited to the hybridoma technique originally developed by Kohler and Milstein (132), as well as the trioma technique, the human B-cell hybridoma technique (133); Cote et al. (134), and the EBV-hybridoma technique to produce human monoclonal antibodies (135). In an additional embodiment of the invention, monoclonal antibodies can be produced in germ-free animals [International Patent Publication No. WO 89/12690, published Dec. 28, 1989]. In fact, according to the invention, techniques developed for the production of “chimeric antibodies” (136-138) by splicing the genes from a mouse antibody molecule specific for an RGS18 polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies [U.S. Pat. Nos. 5,476,786 and 5,132,405 to Huston; U.S. Pat. No. 4,946,778] can be adapted to produce RGS18 polypeptide-specific single chain antibodies. An additional embodiment of the invention utilizes the techniques described for the construction of Fab expression libraries (139) to allow rapid and easy identification of monoclonal Fab fragments with the desired specificity for an RGS18 polypeptide, or its derivatives, or analogs.

Antibody fragments that contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies that recognize a specific epitope of an RGS18 polypeptide, one may assay generated hybridomas for a product that binds to an RGS18 polypeptide fragment containing such epitope. For selection of an antibody specific to an RGS18 polypeptide from a particular species of animal, one can select on the basis of positive binding with an RGS18 polypeptide expressed by or isolated from cells of that species of animal.

The foregoing antibodies can be used in methods known in the art relating to the localization and activity of an RGS18 polypeptide, e.g., for Western blotting, RGS18 polypeptide in situ, measuring levels thereof in appropriate physiological samples, etc. using any of the detection techniques mentioned above or known in the art.

In a specific embodiment, antibodies that agonize or antagonize the activity of an RGS18 polypeptide can be generated. Such antibodies can be tested using the assays described infra for identifying ligands.

The present invention relates to an antibody directed against a polypeptide comprising an amino acid sequence of

1) any one of SEQ ID NOs: 7, 8, 12, or 20,

2) a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20,

3) a polypeptide fragment or variant of a polypeptide comprising an amino acid sequence of either one of SEQ ID NOs: 12 or 20, wherein the polypeptide fragment or variant comprises amino acids a) 1-58 of SEQ ID NO: 12, b) 1-166 of SEQ ID NO: 20, c) 86-202 of SEQ ID NO: 20, or d) 86-166 of SEQ ID NO: 20, or

4) a polypeptide termed “homologous” to a polypeptide comprising an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20,

as produced in the trioma technique or the hybridoma technique described by Kozbor et al. (131) also forms part of the invention.

The invention also relates to single-chain Fv antibody fragments (ScFv) as described in U.S. Pat. No. 4,946,778 or by Martineau et al. (1998).

The antibodies according to the invention also comprise antibody fragments obtained with the aid of phage libraries (140) or humanized antibodies (142, 143).

The antibody preparations according to the invention are useful in immunological detection tests intended for the identification of the presence and/or of the quantity of antigens present in a sample.

An antibody according to the invention may comprise, in addition, a detectable marker that is isotopic or nonisotopic, for example fluorescent, or may be coupled to a molecule such as biotin, according to techniques well known to persons skilled in the art.

Thus, another subject of the invention is a method of detecting the presence of a polypeptide according to the invention in a sample, said method comprising the steps of:

a) bringing the sample to be tested into contact with an antibody directed against

1) a polypeptide comprising an amino acid sequence of any one of SEQ ID NOs: 7, 8, 12, or 20,

2) a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20,

3) a polypeptide fragment or variant of a polypeptide comprising an amino acid sequence of either one of SEQ ID NOs: 12 or 20, wherein the polypeptide fragment or variant comprises amino acids a) 1-58 of SEQ ID NO: 12, b) 1-166 of SEQ ID NO: 20, c) 86-202 of SEQ ID NO: 20, or d) 86-166 of SEQ ID NO: 20, or

4) a polypeptide termed “homologous” to a polypeptide comprising an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20, and

b) detecting the antigen/antibody complex formed.

The invention also relates to a box or kit for diagnosis or for detecting the presence of a polypeptide in accordance with the invention in a sample, said box comprising:

a) an antibody directed against

1) a polypeptide comprising an amino acid sequence of any one of SEQ ID NOs: 7, 8, 12 or 20,

2) a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20,

3) a polypeptide fragment or variant of a polypeptide comprising an amino acid sequence of either one of SEQ ID NOs: 12 or 20, wherein the polypeptide fragment or variant comprises amino acids a) 1-58 of SEQ ID NO: 12, b) 1-166 of SEQ ID NO: 20, c) 86-202 of SEQ ID NO: 20, or d) 86-166 of SEQ ID NO: 20, or

4) a polypeptide termed “homologous” to a polypeptide comprising a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20, and

b) a reagent allowing the detection of the antigen/antibody complex formed.

Pharmaceutical Compositions and Therapeutic Methods of Treatment

The invention also relates to a pharmaceutical composition comprising a nucleic acid according to the invention.

The invention also provides pharmaceutical compositions comprising a nucleic acid encoding an RGS18 polypeptide according to the invention and pharmaceutical compositions comprising an RGS18 polypeptide according to the invention intended for the treatment of a disorder or condition associated with platelet activation dysfunction.

The present invention also relates to a new therapeutic approach for the treatment of a disorder or condition associated with platelet activation dysfunction, comprising transferring and expressing in vivo nucleic acids encoding an RGS18 protein according to the invention. Specifically, the present invention provides a new therapeutic approach for the treatment and/or prevention of a disorder or condition associated with platelet activation dysfunction.

Thus, the present invention offers a new approach for the treatment and prevention of a disorder or condition associated with platelet activation dysfunction. Specifically, the present invention provides methods to restore or promote improved platelet activation within a patient or subject.

The subject of the invention is, in addition, a pharmaceutical composition intended for the prevention or treatment of a disorder or condition associated with platelet activation dysfunction, characterized in that the composition comprises a therapeutically effective quantity of the normal RGS18 polypeptide, in particular a polypeptide comprising an amino acid sequence of a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20. In a preferred embodiment, the RGS18 polypeptide comprises an amino acid sequence of SEQ ID NO: 20.

The invention also relates to the use of the RSG18 polypeptide having an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20 for the manufacture of a medicament intended for the prevention of a disorder or condition associated with platelet activation dysfunction.

The invention relates to a pharmaceutical composition for the prevention or treatment of subjects affected by a disorder or condition associated with platelet activation dysfunction, comprising a therapeutically effective quantity of the polypeptide having an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

According to yet another aspect, the subject of the invention is also a preventive or curative therapeutic method of treating a disorder or condition associated with platelet activation dysfunction, wherein such a method comprises a step in which there is administered to a patient a therapeutically effective quantity of the RGS18 polypeptide in said patient, said polypeptide being, where appropriate, combined with one or more physiologically compatible vehicles and/or excipients.

Preferably, a pharmaceutical composition comprising a polypeptide according to the invention will be administered to the patient. Thus, the invention also relates to pharmaceutical compositions intended for the prevention or treatment of a disorder or condition associated with platelet activation dysfunction, characterized in that they comprise a therapeutically effective quantity of a polynucleotide capable of giving rise to the production of an effective quantity of a normal RGS18 polypeptide, in particular of a polypeptide having an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

The subject of the invention is, in addition, pharmaceutical compositions intended for the prevention or treatment of a disorder or condition associated with platelet activation dysfunction, characterized in that they comprise a therapeutically effective quantity of a normal RGS18 polypeptide, in particular of a polypeptide having an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

Such pharmaceutical compositions will be preferably suitable for the administration, for example by the parenteral route, of a quantity of the RGS18 polypeptide ranging from 1 μg/kg/day to 10 mg/kg/day, preferably at least 0.01 mg/kg/day and more preferably between 0.01 and 1 mg/kg/day.

The invention also provides pharmaceutical compositions comprising a nucleic acid encoding an RGS18 polypeptide according to the invention and pharmaceutical compositions comprising an RGS18 polypeptide according to the invention intended for the treatment or prevention of a disorder or condition associated with platelet activation dysfunction, such as arterial thrombosis, myocardial infarction, coronary artery disease, stroke, cerebrovascular disease, unstable angina, deep vein thrombosis, systemic thromboembolism, as well as its use in invasive cardiac procedures for anti-coagulant purposes.

The present invention also relates to a new therapeutic approach for the treatment of a disorder or condition associated with platelet activation dysfunction, comprising transferring and expressing in vivo nucleic acids encoding an RGS18 protein according to the invention. Specifically, the present invention provides a new therapeutic approach for the treatment and/or prevention of a disorder or condition associated with platelet activation dysfunction, such as arterial thrombosis, myocardial infarction, coronary artery disease, stroke, cerebrovascular disease, unstable angina, deep vein thrombosis, systemic thromboembolism, as well as its use in invasive cardiac procedures for anti-coagulant purposes.

Thus, the present invention offers a new approach for the treatment and prevention of a disorder or condition associated linked to the abnormalities of platelet activation. Specifically, the present invention provides methods to increase, reduce, or inhibit platelet activation within a patient or subject.

Consequently, the invention also relates to a pharmaceutical composition intended for the prevention of or treatment of subjects affected by a disorder or condition associated with platelet activation dysfunction, comprising a nucleic acid encoding the RGS18 protein, in combination with one or more physiologically compatible vehicle and/or excipient.

According to a specific embodiment of the invention, a composition is provided for the in vivo production of the RGS18 protein. This composition comprises a nucleic acid encoding the RGS18 polypeptide placed under the control of appropriate regulatory sequences, in solution in a physiologically acceptable vehicle and/or excipient.

Therefore, the present invention also relates to a composition comprising a nucleic acid encoding a polypeptide comprising an amino acid sequence of either one of SEQ ID NOs: 12 or 20, wherein the nucleic acid is placed under the control of appropriate regulatory elements.

The present invention also relates to a composition comprising a nucleic acid encoding a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20, wherein the nucleic acid is placed under the control of appropriate regulatory elements.

Preferably, such a composition will comprise a nucleic acid comprising a polynucleotide sequence of SEQ ID NO: 18 or SEQ ID NO: 19, placed under the control of appropriate regulatory elements. More preferably, such a composition will comprise a nucleic acid comprising a polynucleotide sequence of nucleotides 163-870 of SEQ ID NO: 19, wherein the nucleic acid is placed under the control of appropriate regulatory elements.

According to another aspect, the subject of the invention is also a preventive or curative therapeutic method of treating a disorder or condition associated with platelet activation dysfunction, wherein such method comprises a step in which there is administered to a patient a nucleic acid encoding an RGS18 polypeptide according to the invention in said patient, said nucleic acid being, where appropriate, combined with one or more physiologically compatible vehicles or excipients.

The invention also relates to a pharmaceutical composition intended for the prevention of or treatment of subjects affected by a disorder or condition associated with platelet activation dysfunction, comprising a recombinant vector according to the invention, in combination with one or more physiologically compatible vehicles or excipients.

According to a specific embodiment, a method of introducing a nucleic acid according to the invention into a host cell, in particular a host cell obtained from a mammal, in vivo, comprises a step during which a preparation comprising a pharmaceutically compatible vector and a “naked” nucleic acid according to the invention, placed under the control of appropriate regulatory sequences, is introduced by local injection at the level of the chosen tissue, for example a smooth muscle tissue, the “naked” nucleic acid being absorbed by the cells of this tissue.

The invention also relates to the use of a nucleic acid according to the invention, encoding the RGS18 protein, for the manufacture of a medicament intended for the prevention or treatment of, or more particularly for the treatment of subjects affected by a disorder or condition associated with platelet activation dysfunction.

The invention also relates to the use of a recombinant vector according to the invention, comprising a nucleic acid encoding the RGS18 protein, for the manufacture of a medicament intended for the prevention of, or more particularly for the treatment of subjects affected by a disorder or condition associated with platelet activation dysfunction.

As indicated above, the present invention also relates to the use of a defective recombinant virus according to the invention for the preparation of a pharmaceutical composition for the treatment and/or prevention of a disorder or condition associated with platelet activation dysfunction.

The invention relates to the use of such a defective recombinant virus for the preparation of a pharmaceutical composition intended for the treatment and/or for the prevention of a disorder or condition associated with platelet activation dysfunction. Thus, the present invention also relates to a pharmaceutical composition comprising one or more defective recombinant viruses according to the invention.

The present invention also relates to the use of cells genetically modified ex vivo with a virus according to the invention, or of producing cells such as viruses, implanted in the body, allowing a prolonged and effective expression in vivo of a biologically active RGS18 protein.

The present invention shows that it is possible to incorporate a nucleic acid encoding an RGS18 polypeptide into a viral vector, and that these vectors make it possible to effectively express a biologically active, mature form. More particularly, the invention shows that the in vivo expression of RGS18 may be obtained by direct administration of an adenovirus or by implantation of a producing cell or of a cell genetically modified by an adenovirus or by a retrovirus incorporating such a nucleic acid.

Preferably, the pharmaceutical compositions of the invention comprise a pharmaceutically acceptable vehicle or physiologically compatible excipient for an injectable formulation, in particular for an intravenous injection, such as for example into the patient's portal vein. These may relate in particular to isotonic sterile solutions or dry, in particular, freeze-dried, compositions, which upon addition depending on the case of sterilized water or physiological saline, allow the preparation of injectable solutions. Direct injection into the patient's portal vein is preferred because it makes it possible to target the infection at the level of the liver and thus to concentrate the therapeutic effect at the level of this organ.

A “pharmaceutically acceptable vehicle or excipient” includes diluents and fillers that are pharmaceutically acceptable for method of administration, are sterile, and may be aqueous or oleaginous suspensions formulated using suitable dispersing or wetting agents and suspending agents. The particular pharmaceutically acceptable carrier and the ratio of active compound to carrier are determined by the solubility and chemical properties of the composition, the particular mode of administration, and standard pharmaceutical practice.

Any nucleic acid, polypeptide, vector, or host cell of the invention will preferably be introduced in vivo in a pharmaceutically acceptable vehicle or excipient. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulator) agency of the Federal or a state government or listed in the U.S. Pharmacopeia or other generally recognized pharmacopeia for use in animals, and more particularly in humans. The term “excipient” refers to a diluent; adjuvant, excipient, or vehicle with which the compound is administered. Such pharmaceutica carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as excipients, particularly for injectable solutions. Suitable pharmaceutical excipients are described in “Remington's Pharmaceutical Sciences” by E. W. Martin.

The pharmaceutical compositions according to the invention may be equally well administered by the oral, rectal, vaginal, parenteral, intravenous, subcutaneous or intradermal route.

The invention also relates to the use of the RGS18 polypeptide having an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20 for the manufacture of a medicament intended for the prevention of, or more particularly for the treatment of a patient or subject affected by a disorder or condition associated with platelet activation dysfunction.

The invention finally relates to a pharmaceutical composition for the prevention or treatment of a patient or subject affected by a disorder or condition associated with platelet activation dysfunction, comprising a therapeutically effective quantity of a polypeptide having an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20, combined with one or more physiologically compatible vehicles and/or excipients.

According to another aspect, the subject of the invention is also a preventive or curative therapeutic method of treating a disorder or condition associated with platelet activation dysfunction, wherein such method comprises a step in which there is administered to a patient or subject a nucleic acid encoding an RGS18 polypeptide in said patient, said nucleic acid being, where appropriate, combined with one or more physiologically compatible vehicles and/or excipients.

According to yet another aspect, the subject of the invention is also a preventive or curative therapeutic method of treating a disorder or condition associated with platelet activation dysfunction, wherein such method comprises a step in which there is administered to a patient or subject a therapeutically effective quantity of an RGS18 polypeptide according to the invention in said patient or subject, said polypeptide being, where appropriate, combined with one or more physiologically compatible vehicles and/or excipients.

The invention relates to a pharmaceutical composition for the prevention or treatment of a patient or subject affected by a dysfunction in platelet activation, comprising a therapeutically effective quantity of a polypeptide having an amino acid sequence of either on of SEQ ID NOs: 12 or 20, or a polypeptide comprising amino acids a) 1-58 of SEQ ID NO: 12, b) 1-166 of SEQ ID NO: 20, c) 86-202 of SEQ ID NO: 20, or d) 86-166 of SEQ ID NO: 20, combined with one or more physiologically compatible vehicles and/or excipients.

According to a specific embodiment, a method of introducing a nucleic acid according to the invention into a host cell, in particular a host cell obtained from a mammal, in vivo, comprises a step during which a preparation comprising a pharmaceutically compatible vector and a “naked” nucleic acid according to the invention, placed under the control of appropriate regulatory sequences, is introduced by local injection at the level of the chosen tissue, for example a smooth muscle tissue, the “naked” nucleic acid being absorbed by the cells of this tissue.

According to yet another aspect, the subject of the invention is also a preventive or curative therapeutic method of treating a disorder or condition associated with platelet activation dysfunction, wherein such method comprises a step in which there is administered to a patient a therapeutically effective quantity of an RGS18 polypeptide according to the invention in said patient, said polypeptide being, where appropriate, combined with one or more physiologically compatible vehicles and/or excipients.

Preferably, a pharmaceutical composition comprising an RGS18 polypeptide according to the invention will be administered to the patient. Thus, the invention also relates to pharmaceutical compositions intended for the prevention or treatment of a disorder or condition associated with platelet activation dysfunction, characterized in that they comprise a therapeutically effective quantity of a nucleic acid encoding an RGS18 polypeptide, in particular an RGS18 polypeptide having an amino acid sequence of either one of SEQ ID NOs: 12 or 20. In a specific embodiment, the RGS18 polypeptide comprises amino acids a) 1-58 of SEQ ID NO: 12, b) 1-166 of SEQ ID NO: 20, c) 86-202 of SEQ ID NO: 20, or d) 86-166 of SEQ ID NO: 20.

The subject of the invention is, in addition, pharmaceutical compositions intended for the prevention or treatment of a disorder or condition associated with platelet activation dysfunction, characterized in that they comprise a therapeutically effective quantity of an RGS18 polypeptide, in particular of a polypeptide comprising an amino acid sequence of either one of SEQ ID NOs: 12 or 20. In a specific embodiment, the RGS18 polypeptide comprises amino acids a) 1-58 of SEQ ID NO: 12, b) 1-166 of SEQ ID NO: 20, c) 86-202 of SEQ ID NO: 20, or d) 86-166 of SEQ ID NO: 20.

In another embodiment, the nucleic acids, polypeptides, recombinant vectors, and compositions according to the invention can be delivered in a vesicle, in particular a liposome (see 144-146).

In yet another embodiment, the nucleic acids, polypeptides, recombinant vectors, recombinant cells, and compositions according to the invention can be delivered in a controlled release system. For example, the polypeptide may be administered using intravenous infusion, an implantable osmotic pump, a transdermal patch, liposomes, or other modes of administration. In one embodiment, a pump may be used (see 144 and 147-149). In another embodiment, polymeric materials can be used (150-155). In yet another embodiment, a controlled release system can be placed in proximity of the target tissue or organ, i.e., the cardiovascular system, thus requiring only a fraction of the systemic dose (see 156). Other controlled release systems that may be employed are discussed in the review by Langer (144).

In a further aspect, recombinant cells that have been transformed with a nucleic acid according to the invention and that express high levels of an RGS18 polypeptide according to the invention can be transplanted in a subject in need of RGS18 polypeptide. Preferably autologous cells transformed with an RGS18 encoding nucleic acid according to the invention are transplanted to avoid rejection; alternatively, technology is available to shield non-autologous cells that produce soluble factors within a polymer matrix that prevents immune recognition and rejection.

Thus, the RGS18 polypeptide can be delivered by intravenous, intraarterial, intraperitoneal, intramuscular, or subcutaneous routes of administration. Alternatively, the RGS18 polypeptide, properly formulated, can be administered by nasal or oral administration. A constant supply of RGS18 can be ensured by providing a therapeutically effective dose (i.e., a dose effective to induce metabolic changes in a subject) at the necessary intervals, e.g., daily, every 12 hours, etc. These parameters will depend on the severity of the disease condition being treated, other actions, such as diet modification, that are implemented, the weight, age, and sex of the subject, and other criteria, which can be readily determined according to standard good medical practice by those of skill in the art.

A subject in whom administration of the nucleic acids, polypeptides, recombinant vectors, recombinant host cells, and compositions according to the invention is performed is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Preferably, a pharmaceutical composition comprising a nucleic acid, a recombinant vector, a polypeptide, or a recombinant host cell, as defined above, will be administered to the patient or subject.

Methods of Screening an Agonist or Antagonist Compound for the RGS18 Polypeptide

The invention also relates to methods for the detection of activators or inhibitors of RGS18 protein and RGS18 domain comprising polypeptides.

The invention also provides methods for screening small molecules and compounds that act on the RGS18 protein to identify agonists and antagonists of RGS18 polypeptide that can increase, reduce, or inhibit platelet activation from a therapeutic point of view. These methods are useful to identify small molecules and compounds for therapeutic use in the treatment of a disorder or condition associated with platelet activation dysfunction.

According to another aspect, the invention also relates to various methods of screening compounds or small molecules for therapeutic use that are useful in the treatment of a disorder or condition associated with platelet activation dysfunction, such as arterial thrombosis, myocardial infarction, coronary artery disease, stroke, cerebrovascular disease, unstable angina, deep vein thrombosis, systemic thromboembolism, as well as its use in invasive cardiac procedures for anti-coagulant purposes.

Therefore, the invention also relates to the use of an RGS18 polypeptide or a cell expressing an RGS18 polypeptide according to the invention, for screening active ingredients for the prevention or treatment of a disorder or condition associated with platelet activation dysfunction. The catalytic sites and oligopeptide or immunogenic fragments of an RGS18 polypeptide can serve for screening product libraries by a whole range of existing techniques. The polypeptide fragment used in this type of screening may be free in solution, bound to a solid support, at the cell surface or in the cell. The formation of the binding complexes between the RGS18 polypeptide fragments and the tested agent can then be measured.

Another product screening technique that may be used in high-flux screenings giving access to products having affinity for the protein of interest is described in application WO84/03564. In this method, applied to an RGS18 protein, various products are synthesized on a solid surface. These products react with the RGS18 protein or fragments thereof and the complex is washed. The products binding the RGS18 protein are then detected by methods known to persons skilled in the art. Non-neutralizing antibodies can also be used to capture a peptide and immobilize it on a support.

Another possibility is to perform a product screening method using an RGS18 neutralizing antibody competition, an RGS18 protein and a product potentially binding the RGS18 protein. In this manner, the antibodies may be used to detect the presence of a peptide having a common antigenic unit with an RGS18 polypeptide or protein.

Accordingly, this invention relates to the use of any method of screening products, i.e., compounds, small molecules, and the like, this being in all synthetic or cellular types, that is to say of mammals, insects, bacteria, or yeasts expressing constitutively or having incorporated a human RGS18 encoding nucleic acid.

The present invention also relates to the use of such a system for screening molecules that modulate the activity of the RGS18 protein. Thus, the invention relates to methods of screening and identifying a modulator, agonist, or antagonist of an RGS18 polypeptide in a sample.

The present invention relates to methods of identifying a modulator, agonist, or antagonist of an RGS18 polypeptide in a sample comprising

a) incubating a labeled GTP-loaded G protein polypeptide with an RGS18 polypeptide with the sample;

b) measuring the rate or extent of GTP hydrolysis; and

c) comparing the Tate or extent of GTP hydrolysis determined in step b) with a rate or extent of GTP hydrolysis measured with a reconstituted labeled GTP-loaded G protein polypeptide/RGS18 poylpeptide mixture that has not been previously incubated in the presence of the sample.

In a specific embodiment, the labeled GTP-loaded G protein polypeptide of step a) is loaded with γ-³²P-GTP and the rate or extent of GTP hydrolysis of step b) is measured by determining the amount of free ³²Pᵢ released.

In another specific embodiment, the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.

In particular, the invention relates to a method of identifying a modulator, agonist, or antagonist of an RGS18 polypeptide in a sample, wherein the method comprises

a) loading a purified G protein polypeptide with γ-³²P-GTP;

b) incubating the γ-³²P-GTP-loaded G protein polypeptide of step a) with a purified RGS18 poylpeptide and a candidate modulator, agonist, or antagonist compound for the RSG18 polypeptide;

c) measuring the rate or extent of GTP hydrolysis by determining the amount of free ³²Pᵢ released; and

d) comparing the rate or extent of GTP hydrolysis determined in step c) with a rate or extent of GTP hydrolysis measured with a reconstituted γ-³²P-GTP-loaded G protein polypeptide/purified RGS18 poylpeptide mixture that has not been previously incubated in the presence of the candidate modulator, agonist, or antagonist compound for the RGS18 polypeptide.

Reconstitution of purified RGS18 polypeptide with purified G protein polypeptides within the methods of the invention may be performed according to any technique, in particular, according to the technique described by Berman et al. (157).

In a first specific embodiment, the RGSI 8 polypeptide comprises either one of SEQ ID NOs: 12 or 20.

In a second specific embodiment, the RSG18 polypeptide comprises amino acids a) 1-58 of SEQ ID NO: 12, b) 1-166 of SEQ ID NO: 20, c) 86-202 of SEQ ID NO: 20, or d) 86-166 of SEQ ID NO: 20.

An RGS18 modulator compound identified by the methods of the invention may reduce or increase the rate or extent of GTP hydrolysis by an RGS18 polypeptide according to the invention.

The present invention relates to methods of identifying a modulator, agonist, or antagonist of an RGS18 polypeptide in a sample comprising

a) incubating a cell membrane fraction expressing an RGS18 polypeptide with a labeled GTP and the sample;

b) measuring the rate or extent of GTP hydrolysis; and

c) comparing the rate or extent of GTP hydrolysis determined in step b) with a rate or extent of GTP hydrolysis measured with a cell membrane fraction expressing an RGS18 polypeptide that has not been previously incubated in the presence of the sample.

In a specific embodiment, the cell membrane fraction is obtained from a cell that, either naturally or after transfecting the cell with an RGS18 encoding nucleic acid, expresses an RGS18 polypeptide, and isolating the cell's membrane.

In another specific embodiment, the labeled GTP of step a) is labeled with γ-³²P and the rate or extent of GTP hydrolysis of step b) is measured by determining the amount of free ³²Pᵢ released.

In another specific embodiment, the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.

The present invention also relates to a method of identifying a modulator, agonist, or antagonist of an RGS18 polypeptide in a sample, wherein the method comprises

a) obtaining a cell, for example a cell line, that, either naturally or after transfecting the cell with an RGS18 encoding nucleic acid, expresses an RGS18 polypeptide, and isolating the cell's membrane;

b) incubating the cell membrane of step a) with γ-³²P-GTP and a candidate modulator, agonist, or antagonist compound for the RSG18 polypeptide;

c) measuring the rate or extent of GTP hydrolysis by determining the amount of free ³²Pᵢ released; and

d) comparing the rate or extent of GTP hydrolysis determined in step c) with a rate or extent of GTP hydrolysis measured with a cell membrane that has not been previously incubated in the presence of the candidate modulator, agonist, or antagonist compound for the RGS18 polypeptide.

The cell membrane fractions within the methods of the invention may be prepared according to any technique, in particular, according to the technique described by Denecke et al. (158).

In a first specific embodiment, the RGS18 polypeptide comprises either one of SEQ ID NOs: 12 or 20.

In a second specific embodiment, the RSG18 polypeptide comprises amino acids a) 1-58 of SEQ ID NO: 12, b) 1-166 of SEQ ID NO: 20, c) 86-202 of SEQ ID NO: 20, or d) 86-166 of SEQ ID NO: 20.

An RGS18 modulator compound identified by the methods of the invention may reduce (inhibitor) or increase (activator) the rate or extent of GTP hydrolysis by an RGS18 polypeptide according to the invention.

The present invention also relates to a method of identifying a modulator, agonist, or antagonist of an RGS18 polypeptide, wherein the method comprises determining the effects of the modulator, agonist, or antagonist on downstream effects or effector molecules of the RGS18 polypeptide.

Therefore, the present invention relates to methods of identifying a modulator, agonist, or antagonist of an RGS18 polypeptide in a sample comprising

a) incubating a cell expressing an RGS18 polypeptide with a labeled adenine and the sample;

b) measuring the amount of labeled cyclic AMP (cAMP) produced; and

c) comparing the amount of labeled cAMP measured in step b) with an amount of labeled cAMP measured with a cell expressing an RGS18 polypeptide that has not been previously incubated in the presence of the sample.

In a specific embodiment, the cell expressing the RGS18 polypeptide is transfected with an RGS18 encoding nucleic acid.

In another specific embodiment, the labeled adenine of step a) is ³H-adenine.

In another specific embodiment, the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.

In particular, the present invention relates to a method of identifying a modulator, agonist, or antagonist of an RGS18 polypeptide, wherein the method comprises

a) obtaining a cell, for example a cell line, that, either naturally or after transfecting the cell with an RGS18 encoding nucleic acid, expresses an RGS18 polypeptide,

b) incubating the cell of step a) with ³H-adenine and a candidate modulator compound for the RSG18 polypeptide;

c) measuring the amount of radioactively labeled cyclic AMP (cAMP) produced; and

d) comparing the amount of radiactively labeled cAMP measured in step c) with an amount of radiactively labeled cAMP measured with a cell that has not been previously incubated in the presence of the candidate modulator, agonist, or antagonist compound for the RGS18 polypeptide.

The amount of radiactively labeled cAMP may be determined according to any technique, in particular, according to the technique described by Huang et al. (159).

In a first specific embodiment, the RGS18 polypeptide comprises either one of SEQ ID NOs: 12 or 20.

In a second specific embodiment, the RSG18 polypeptide comprises amino acids a) 1-58 of SEQ ID NO: 12, b) 1-166 of SEQ ID NO: 20, c) 86-202 of SEQ ID NO: 20, or d) 86-166 of SEQ ID NO: 20.

An RGS18 modulator compound identified by the methods of the invention may reduce or increase the amount of cAMP produced by an RGS18 polypeptide according to the invention.

The present invention also relates to methods of identifying a modulator, agonist, or antagonist of an RGS18 polypeptide in a sample comprising

a) incubating a cell expressing an RGS18 polypeptide with a labeled inositol and the sample;

b) measuring the amount of labeled inositol triphosphate produced; and

c) comparing the amount of labeled inositol triphosphate measured in step b) with an amount of labeled inositol triphosphate measured with a cell expressing an RGS18 polypeptide that has not been previously incubated in the presence of the sample.

In a specific embodiment, the cell expressing the RGS18 polypeptide is transfected with an RGS18 encoding nucleic acid.

In another specific embodiment, the labeled inositol of step a) is ³H-inositol.

In another specific embodiment, the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.

In particular, the present invention also relates to a method of identifying a modulator, agonist, or antagonist of an RGS18 polypeptide in a sample, wherein the method comprises

a) obtaining a cell, for example a cell line, that, either naturally or after transfecting the cell with an RGS18 encoding nucleic acid, expresses an RGS18 polypeptide,

b) incubating the cell of step a) with ³H-inositol and a candidate modulator compound for the RSG18 polypeptide;

c) measuring the amount of radioactively labeled inositol triphosphate produced; and

d) comparing the amount of radiactively labeled inositol triphosphate measured in step c) with an amount of radiactively labeled inositol triphosphate measured with a cell that has not been previously incubated in the presence of the candidate modulator, agonist, or antagonist compound for the RGS18 polypeptide.

The amount of radiactively labeled inositol triphosphate may be determined according to any technique, in particular, according to the technique described by Huang et al. (159).

In a first specific embodiment, the RGS18 polypeptide comprises either one of SEQ ID NOs: 12 or 20.

In a second specific embodiment, the RSG18 polypeptide comprises amino acids a) 1-58 of SEQ ID NO: 12, b) 1-166 of SEQ ID NO: 20, c) 86-202 of SEQ ID NO: 20, or d) 86-166 of SEQ ID NO: 20.

An RGS18 modulator compound identified by the methods of the invention may reduce or increase the amount of inositol triphosphate produced by an RGS18 polypeptide according to the invention.

According to a first aspect of the above screening methods, the cells used are cells naturally expressing an RGS18 polypeptide. They may be human platelets in primary culture, purified from a population of human blood mononuclear cells. They may also be human megakaryocytic cell lines, such as HEL cells, Meg-01 cells, and Dami cells.

According to a second aspect, the cells used in the screening methods described above may be cells not naturally expressing, or alternatively expressing at a low level, an RGS18 polypeptide, said cells being transfected with a recombinant vector according to the invention capable of directing the expression of a nucleic acid encoding an RGS18 polypeptide.

According to a third aspect of the above screening methods, the RGS18 polypeptide may be a recombinant RGS18 polypeptide.

The present invention may be better understood by reference to the following non-limiting Examples, which are provided as exemplary of the invention.

### EXAMPLES

In an effort to better understand modulation of GPCR-mediated signaling in platelets, Applicants sought to identify Regulators of G protein signaling proteins (RGSs) that are present in human platelets and several megakaryocytic cell lines. Using degenerate oligonucleotides based on conserved regions of the highly homologous RGS domain, RT-PCR was performed using human platelet RNA, as well as RNA from several megakaryocytic cell lines. In addition to confirming the presence of several known RGS transcripts, a novel RGS domain containing transcript was found in platelet RNA. Northern blot analysis of multiple human tissues indicates that this novel transcript is most abundantly expressed in platelets compared to other tissues examined. This RGS transcript is abundantly expressed in platelets, with significantly lower expression in other tissues, primarily those of the hematopoetic system. This transcript is modestly expressed in three megakaryocyte cell lines and tissues of hematopoetic origin such as leukocytes, bone marrow and spleen with low level expression detected in other tissues as well. Full-length cloning of this novel RGS, which has been termed RGS18, demonstrates that this transcript encodes a 235 amino acid protein. RGS18 is most closely related to RGS5 (46% identity) and has ˜30-40% identity to other RGS proteins. Peptide-directed antisera against RGS18 detect the expression of ˜30 kDa protein in platelet, leukocyte and megakaryocyte cell line lysates. In vitro RGS18 binds to endogenous G_{αi2}, G_{αi3} and G_{αq} but not G_{αz}, G_{αs} or G_{α12} from GDP+AlF₄⁻-treated platelet lysates. Since platelet aggregation requires activation of a receptor coupled to G_{αq} and/or one or more forms of G_{αi}, RGS18 may be responsible in part for regulation of pathways important to platelet activation.

Reagents were obtained from Sigma (St. Louis, Mo.) unless otherwise noted. Reagents for GST fusion protein expression and purification including pGEX-5X-1 and glutathione-Sepharose 4B were obtained from Amersham/Pharmacia (Piscataway, N.J.). Oligonucleotides and peptides were produced by the Core Biotechnologies Department at Rhone-Poulenc Rorer. Double-stranded cDNA from human leukocytes and human bone marrow was obtained from Clontech (Palo Alto, Calif.). Marathon cDNA from human peripheral blood leukocytes and bone marrow, and the Advantage PCR kit for 5′ Race were from Clontech (Palo Alto, Calif.). All reagents for cell culture were obtained from Gibco/BRL (Rockville, Md.). Reagents for Western blotting including goat anti-rabbit IgG-coupled to HRP were purchased from BioRad (Hercules, Calif.). SuperSignal Pico Reagent was purchased from Pierce (Rockford, Ill.). Complete EDTA-free protease inhibitor cocktail was from Roche Biochemicals (Indianapolis, Ind.). Cell lines were obtained from the ATCC (Manassas, Va.).

### Example 1

### RT-PCR Amplification and Cloning of Nucleic Acids Encoding an RGS Domain

Preparation of Platelets, Leukocytes and Cell Lines: HEL cells and Meg-01 cells were maintained in RPMI supplemented with 10% fetal bovine serum, 0.3 mg/ml L-glutamine and 100 U/ml penicillin G/100 mg/ml streptomycin sulfate. Dami cells were grown in Iscove's Modified Dulbecco's Medium supplemented with 10% heat-inactivated horse serum, 0.3 mg/ml L-glutamine and 100 U/ml penicillin G/100 mg/ml streptomycin sulfate. Human platelets were obtained from Interstate Blood Bank (Memphis, Term.) in concentrate form and were used on the day after collection (Day 2). When necessary for functional studies fresh platelets were drawn from consenting donors using 0.38% citrate as the anti-coagulant. For both, platelets were spun at 120×g for 15 minutes to pellet the red cells and retrieve the platelet rich plasma (PRP). Prostaglandin E₁ (0.5 mg/ml) was added to the PRP to prevent activation, and the platelets were pelleted at 800×g for 15 minutes. The platelet pellet was washed once in Tyrodes buffer (137 mM NaCl, 2.7 mM KCl, 1 mM MgCl₂-6H₂O, 5.5 mM glucose, 11.9 mM NaHCO₃, 0.36 MM NaH₂PO₄H₂O, 10 mM HEPES pH, 7.35) plus 0.35% human serum albumin (HSA; this was reduced to 0.1% HSA for protein lysates to reduce albumin protein carry over) plus 0.25 mg/ml prostaglandin E₁. Platelets were pelleted at 800×g and resuspended in the appropriate buffer (Trizol for RNA, Lysis buffer for protein lysates). White blood cells were obtained fresh from consenting donors and isolated during platelet preparation. Briefly, after removal of the PRP for platelet isolation, the buffy coat, which is the white layer of cells that forms an interface between the platelet rich plasma and the pelleted red blood cells, was removed and brought up to 15 mls with platelet-poor plasma and diluted 1:1 with PBS. Fifteen mls of this was carefully layered on top of 15 ml of Hipaque. This was spun at 400×g for 30 minutes at room temperature. A band containing the human leukocytes was isolated, diluted 1:1 in PBS and spun at 400×g for 10 minutes to isolate the cells. The cell pellet was then resuspended in the appropriate lysis buffer (Trizol or protein lysis buffer).

RT-PCR cloning Strategy: Four degenerate oligonucleotides were synthesized which have been used previously for isolating RGS transcripts and are designed in regions of high homology flanking the RGS domain of several members of the RGS superfamily (8). These four oligonucleotides given in IUB code are RGS1: GRIGARAAYHTIGARTTYTGG (SEQ ID No: 1), RGS2: GRIGARAAYHTIMGITTYTGG (SEQ ID No: 2), RGS3: GRTAIGARTYITTYTYCAT (SEQ ID No: 3), and RGS4: GRTARCTRTYITYTYCAT (SEQ ID No: 4). Total RNA was prepared from human platelets, HEL cells, DAMI cells and MEG-01 cells using Trizol Reagent (Gibco/BRL, Rockville, Md.) according to the manufacturer's instructions. Human platelets obtained from Day 2 platelet concentrate (Interstate Blood Bank, Memphis, Tenn.) were prepared as above, pelleted and lysed in 5 ml Trizol reagent. Platelet counts varied in the cell pellet but typical values ranged between 0.9-5×10¹⁰ platelets and the yield of total RNA was between 25-100 mg. Single strand cDNA was prepared by reverse transcription of total RNA using Superscript II (Gibco/BRL, Rockville, Md.), 2-5 mg of total RNA and 160 ng of random hexamer (Roche Biochemicals, Indianapolis, Ind.) following the manufacturer's instructions. PCR was performed using ⅛ of the cDNA reaction, typically 5 μl in a 50 μl reaction volume containing 300 ng of each of the oligonucleotides RGS1, RGS2, RGS3 and RGS4, 400 mM dNTPs, 1×PCR buffer with 1.5 mM MgCl₂ and 1 μl of Taq polymerase (Gibco/BRL, Rockville, Md.). In some studies, 1 μl of human bone marrow human peripheral blood leukocyte cDNA was used for amplification. Amplification was performed in a PCRSprint Thermocycler (Hybaid, Franklin, Mass.) with the following cycling conditions, 94° C. for 2 minutes for 1 cycle, 94° C. for 30 seconds, 42° C. for 1 minute, 72° C. for 2 minutes for 35 cycles followed by an extension at 72° C. for 7 minutes. In order to increase the yield of colonies, a second round of amplification was performed using 1-5 μl of the initial PCR reaction product. Reaction products (˜230 bp) were separated by agarose gel electrophoresis and purified using the Qiaquick gel extraction kit (Qiagen, Chatsworth, Calif.). The resulting purified PCR reactions were subcloned into pCR2.1 using the TA cloning kit (Invitrogen, Carlsbad, Calif.) and transformed to INVαF-cells. Single colonies were replicated to fresh plates and numbered for reference and grown for DNA purification. Automated plasmid purification and sequencing was performed on all the colonies (50 colonies when two rounds of amplification were performed, 25 for one) using universal primers complementary to the cloning vector sequence (M13 Forward Primer, SEQ ID NO: 37 and M13 Reverse Primer, SEQ ID NO: 38). Sequence analysis was performed using an automated sequencing system (ABI). The identity of each PCR product was determined by comparing sequence data to that of known RGSs using Seqman and Megalign programs of the Lasergene software and BESTFIT, GAP and PILEUP of the GCG program.

Results of RT-PCR of RGSs from Human Platelets and Megakaryocytic Cell Lines.

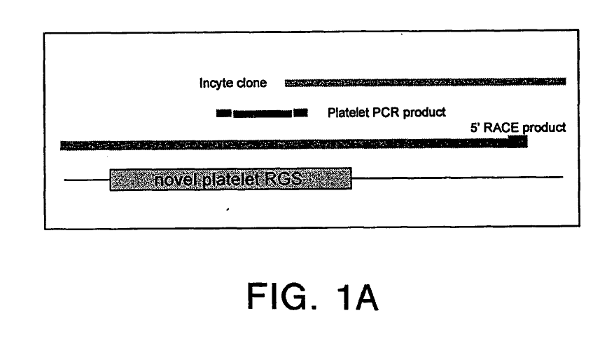

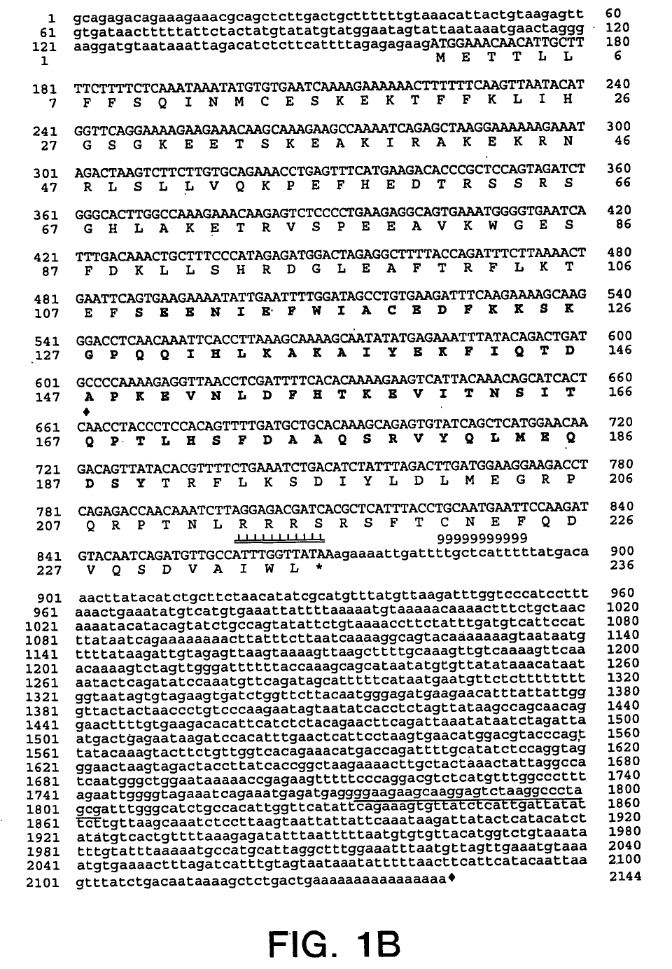

A Reverse Transcription-Polymerase Chain Reaction (RT-PCR) strategy was employed to identify RGS family members expressed in human platelets and several megokaryocytic cell lines. Degenerate primers were designed and synthesized based on regions that are highly homologous in several of the previously identified RGS proteins (8). These primers were used to amplify total RNA from human platelets (Plt), and three megakaryocytic cell lines, DAMI, HEL, and MEG-01 cells. Amplification of RNA from all four tissues resulted in a band of ˜240 base pairs, which was purified and subcloned into pCR2.1 and transformed to competent Escherichia coli. Fifty colonies from each PCR reaction were picked, grown and plasmid was isolated from each and sequenced. Analysis of the resulting sequence data by comparison to known RGS proteins was performed and the results are summarized in Table 1. The most predominate amplification product in all three megakaryocytic cell lines is RGS16, an RGS first identified in mouse retina (160) but also present in lymphocytes and many other tissues (161, 162). In contrast to the cell lines however, the most predominate PCR product (SEQ ID NO: 11) from platelet RNA encodes a novel partial RGS domain (SEQ ID NO: 12 encoded by nucleotides 1-241 of SEQ ID NO: 11), which is not present in the public domain databases. A single colony out of the 50 examined in DAMI cells also encoded this novel RGS domain. This novel partial RGS domain comprises amino acids 1 to 81 of SEQ ID NO: 12 and is shown in FIG. 1, slightly smaller due to position of degenerate oligonucleotide probes. This novel RGS domain displays 3046% homology to the known RGSs, with the highest degree of homology to RGS5. Additionally, platelets contain transcript for RGS10, and MEG-01 cells contain RGS4. Not surprisingly all tissues tested contain the ubiquitously expressed RGS2.

TABLE 1
Identification of RGS isoforms by degenerate RT-PCR of
total RNA from human platelets and three megakaryocytic cell lines.

| | Plt | DAMI | HEL | MEG |
|---|---|---|---|---|
| | RNA | RNA | RNA | RNA |
| RGS2 | 3 | 1 | 1 | 3 |
| RGS10 | 1 | | | |
| RGS16 | 4 | 33 | 37 | 20 |
| RGS4 | 3 | | | |
| novel RGS18 | 17 | 1 | | |
| vector/? | 12/2 | 4/4 | 7/2 | 14/0 |
| ND | 11 | 7 | 3 | 10 |
| Degenerate oligonucleotide primers designed against conserved regions of the RGS domain were used to amplify RNA from human platelets, DAMI, HEL and MEG-01 cells as described above. The amplification products were blunt-ligated to pCR2.1 and transformed into competent Escherichia coli. Fifty colonies were picked and plasmid DNA was isolated and subjected to sequence analysis. The resultant sequence data from each colony was compared to the coding regions of known RGSs and | | | | |
| # tabulated. In some cases the plasmid religated to itself resulting in no insert, these are noted as “vector”. Plasmids which contained inserts but had no apparent homology to a known gene are labeled “?”, and probably are artifacts of the PCR reaction. Sequencing reactions that resulted in poor quality data (numerous unknown nucleotides) are noted as “ND” for not determined. | | | | |

### Example 2

### Full-Length Cloning of RGS18 cDNA

The novel PCR product was compared to the LIFESEQ proprietary Incyte database of ESTs. Only one match was found. This EST was from a human thyroid library and the 5′ most end of the EST was virtually identical to the last 72 base pairs of our clone. This EST clone was purchased from Incyte, grown, and analyzed to determine whether these two transcripts were indeed related. Complete sequence analysis using M13 Forward and M13 Reverse Primers (SEQ ID NOs: 37 and 38, respectively) confirmed the presence of the 72 bp overlap and provided us with sequence information for the carboxy-terminal portion of the protein and a 1274 bp 3′ untranslated region.

Full-length sequence information was obtained using 5′ RACE. Marathon cDNA from human bone marrow and peripheral blood leukocyte (Clontech, Palo Alto, Calif.) was used for the 5′ RACE strategy. 5′ Race was carried out with the antisense primer [5′-cgctagggccttagactccttgcttcttcc-3′, SEQ ID NO: 5] from the 3′ untranslated region, using Marathon Ready cDNAs coupled with the Advantage cDNA polymerase (Clontech, Palo Alto, Calif.) following the manufacturer's instructions for 5′ RACE. The reaction products from the RACE procedure were subcloned into pCR2.1 and transformed to competent INVαF⁻ Escherichia coli and 20 colonies were analyzed by restriction analysis and sequencing using nRGS11-nRGS19 sequencing primers (SEQ ID NOs: 30-36, respectively) and M13 Forward and M13 Reverse Primers (SEQ ID NOs: 37 and 38, respectively). Table 2 describes the nRGS11-nRGS19 sequencing primers used to determine the nucleic acid sequence of the 5′ RACE clones and the location of these primers within SEQ ID NO: 19, a cDNA encoding the full length RGS18 polypeptide.

TABLE 2
5′ RACE RGS18-Specific Sequencing Primers

| | | SEQ ID | LOCATION IN |
|---|---|---|---|
| PRIMER | SEQUENCE (5′→3′) | NO: | SEQ ID NO: 19 |
| nRGS11 | CTACTATGTATATGTATGGAATAG | 30 | 77-100 |
| nRGS12 | GAATTTTGGATAGCCTGTGAAG | 31 | 502-523 |
| nRGS13 | CGATCACGCTCATTTACCTGCAAT | 32 | 805-828 |
| nRGS14 | CTGAAATATGTCATGTGAAATTAT | 33 | 964-987 |
| nRGS17 | CATAAACATGCGATATGTTAG | 34 | 918-938 |
| nRGS18 | TGGGGCATCAGTCTGTATAAA | 35 | 586-606 |
| nRGS19 | CCTGAACCATGTATTAACTTG | 36 | 228-248 |

Results of the Full-length Cloning of the Novel Platelet RGS.

In order to identify the full-length sequence of this partial clone, electronic searches of EST databases were performed. No identical hits were found in the public domain EST databases. The novel PCR product was also compared to the LIFESEQ proprietary Incyte database of ESTs. Only one hit was found, an EST (SEQ ID NO: 6) from a human thyroid library. Nucleotides 1-59 of SEQ ID NO: 6 display complete identity to nucleotides 170-228 of SEQ ID NO: 1, and nucleotides 60-72 of SEQ ID NO: 6 display partial identity to nucleotides 229-241 of SEQ ID NO: 1. Since the library was primed with Oligo(dT), it is likely that this EST comprises the carboxy-terminal region and 3′ untranslated domain of the novel RGS18 transcript. This clone was purchased from Incyte and analyzed by restriction analysis and sequencing. The Incyte clone contains a cDNA insert of 1486 bp (SEQ ID NO: 6) which has identity to the novel PCR product (nucleotides 170-228 of SEQ ID NO: 11) at the 5′ end (nucleotides 1-59 of SEQ ID NO: 6) and a stretch of poly(A)⁺ residues at the 3′ end (nucleotides 1471-1486 of SEQ ID NO: 6). When translated, this EST comprises a partial open reading frame (nucleotides 3-209 of SEQ ID NO: 6) encoding a 69 amino acid polypeptide (SEQ ID NO: 13). The partial open reading frame of this EST is contiguous with that of the novel PCR product (SEQ ID NO:11), wherein amino acids 1-23 of SEQ ID NO: 13 are identical to amino acids 59-81 of SEQ ID NO: 12, except for amino acid 20 of SEQ ID NO: 13 (amino acid 78 of SEQ ID NO: 12). Based upon homology to other known RGS domain-containing proteins that extends beyond the 72 bp overlap of the EST and PCR product nucleic acids lends support that the EST represents the 3′ end of the novel RGS18 PCR product. To further confirm that these two cDNAs are in fact from the same transcript, RT-PCR analysis of platelet RNA was performed with a sense primer (5′-ATAGCCTGTGAAGATTTCAAG-3′; SEQ ID NO: 14) designed against near 5′ sequence information from our platelet PCR product and three antisense primers (5′-TGGCAACATCTGATTGTACAT-3′, SEQ ID NO: 15; 5′-AAGTTTGTCATAAAAATGAGC-3′, SEQ ID NO: 16; and 5′-TTAACATAAACATGCGATATG-3′, SEQ ID NO: 17) chosen at three different sites within the Incyte EST beyond the region of overlap with the initial PCR clone. PCR products of the expected size were obtained from each of these reactions confirming that the novel PCR clone and the Incyte EST cDNA are in fact part of a contiguous transcript in platelet RNA (data not shown).

Using sequence information from the 3′ untranslated region of the Incyte clone (SEQ ID NO: 6), 5′ RACE was performed to isolate the entire coding region of the novel RGS18. Since platelets do not contain abundant levels of high quality RNA, cDNAs from human bone marrow and peripheral blood leukocytes were used specifically designed for 5′ RACE. Preliminary Northern blot data demonstrated that the novel RGS18 transcript is present at low levels in both these tissues. A primer (SEQ ID NO: 5) was designed within the far 3′ untranslated region, the location of this primer is depicted by the underlined sequence information in FIG. 1, panel B. 5′ RACE was performed using the Advantage PCR kit according to the manufacturer's instructions with this 3′ untranslated region primer (SEQ ID NO: 5) and both bone marrow and peripheral blood leukocyte cDNA. The 5′ RACE PCR reaction products were subcloned into pCR2.1 and analyzed by restriction mapping. The longest inserts (˜2 kB) were chosen for sequence analysis. Sequence analysis as described above determined that these 5′ RACE PCR products comprised 1840 nucleotides (SEQ ID NO: 18). The 5′ RACE PCR products comprised the entire predicted open reading frame (nucleotides 163-870 of SEQ ID NO: 18) that encodes the novel full length RGS18 polypeptide (SEQ ID NO: 20) and a short stretch of the 5′ untranslated region (nucleotides 1-162 of SEQ ID NO: 18). Sequence data were compiled from the 5′ RACE clone (SEQ ID NO: 18), the Incyte EST cDNA (SEQ ID NO: 6) and the initial PCR clone (SEQ ID NO: 11) and assembled, and a schematic of the relative positions of each of these overlapping clones is shown in FIG. 1, panel A. FIG. 1, Panel B shows the nucleotide sequence of this compiled nucleic acid (SEQ ID NO: 19) and the predicted amino acid sequence of the novel full length RGS18 polypeptide it encodes (SEQ ID NO: 20). Nucleotides 163-870 of SEQ ID NO: 19 represent the full length open reading frame encoding the full length RGS18 polypeptide (SEQ ID NO: 20).

The RGS18 cDNA (SEQ ID NO: 19) is 2144 base pairs in length and encodes a protein of 235 amino acids (SEQ ID NO: 20). The theoretical pI of this protein is 7.73 with a molecular weight of 27, 582.22 daltons. Scanning for protein sequence motifs, using the Prosite database, confirms that this protein contains a RGS domain (residues 86 through 202 of SEQ ID No: 20), as well as putative consensus sites for phosphorylation by several protein kinases. Residues 213 to 216 of SEQ ID No: 20 form a consensus site for phosphorylation by cAMP and cGMP-dependent protein kinase (shown underlined in FIG. 1, Panel B). Four potential sites for protein kinase C phosphorylation and five for casein kinase II (CK II) phosphorylation are present (SEQ ID NO: 20 residues 28-30, 33-35, 63-65 and 92-94 for PKC and SEQ ID NO: 20 residues 28-31, 33-36, 76-79, 92-95 and 220-223 for CK II). Amino acids 221-224 of SEQ ID NO: 20 in the carboxy-terminus of the protein encode a putative CAAX motif that may act as a site of modification by fatty acylation and serve to regulate the activity of RGS18.

### Example 3

### Tissue Distribution of RGS18 Nucleic Acids and Polypeptides

Total RNA Isolation and Northern Blot Analysis: Preparation of total RNA from human platelets, white blood cells, HEL cells, Dami cells and Meg-01 cells was carried out as described above. A multiple human tissue Northern Blot was purchased from Clontech (Palo Alto, Calif.). For Northern blots of human platelets, human leukocytes and the megakaryocytic cells lines, 10 mg of total RNA was run on a 1.5% agarose/5.8% formaldehyde gel in 1×MOPS buffer (20 mM 3-N-Morpholinopropanesulfonic acid, pH 7.0, 5 mM sodium acetate, 1 mM EDTA). RNA was transferred in 20×SSC (0.3 mM NaCl, 0.3 mM sodium citrate) to nylon membrane using the Turboblotter System (Schleicher and Schull, Keene, N.H.) and UV crosslinked to the membrane using a Stratalinker. (Stratagene, La Jolla, Calif.). A multiple tissue Northern blot with 1 mg of poly(A⁺) RNA from each of 12 different tissues was purchased from Clontech (Palo Alto, Calif.). Both of these blots were prehybridized in 10 ml ExpressHyb solution (Clontech, Palo Alto, Calif.) at 68° C. for 1 hour. For each blot, approximately 25 ng of the 1486 base pair Incyte clone EST (SEQ ID NO: 6) encoding the 3′ end of the coding region and the 3′ untranslated region was radiolabeled with ³²P-α[dCTP] and the High Prime Kit (Roche Biochemicals, Indianapolis, Ind.) and purified on a microspin S-200 column (Amersham/Pharmacia, Piscataway, N.J.). Two labeled probe batches were mixed and added to 20 ml of ExpressHyb. Each blot was hybridized with 10 ml of this probe mix for 2 hours at 68° C. This process ensured that the blots were each hybridized with probes of the same specific activities and would facilitate comparison of the results. The blots were washed for 40 minutes in 2×SSC/0.05% SDS at room temperature followed by a high stringency wash in 0.1X SSC/0.1% SDS at 50° C. for 40 minutes and exposed to Kodak BioMax MR film at −70° C. Following removal of the probe with 0.5% SDS at 100° C., normalization of the blots was performed using a labeled β-actin probe (Clontech, Palo Alto, Calif.; catalog #SO130) and performed as above.

Production of Polyclonal Antisera, Western Blot Analysis, and Cell Lysate Preparation: Two peptides were selected to make polyclonal antisera, KLIHGSGEETSKEAKIR (SEQ ID NO: 7) from the amino-terminal portion of RGS18 and QRPTNLRRRSRSFTCNEFQ (SEQ ID NO: 8), from the carboxy-terminal region. These peptides were synthesized in-house by RPR Core Biotechnologies Department and conjugated to Keyhole Limpet hemacyanin (KLH) for injection into rabbits. Custom polyclonal antisera were produced from the conjugated peptides by Rockland Immunochemicals (Gilbertsville, Pa.) according standard procedures. The specificity of the antisera was characterized using platelet lysates and recombinant RGS18 produced by coupled in vitro transcription/translation (TNT reticulolysate system, Promega, Madison, Wis.). Typically, antisera was used in Western blotting at 1:500 (3NRGS-12) or 1:1,000 (5NRGS-13) dilutions in 5% non-fat dry milk in TBST (20 mM Tris-HCl, pH 7.5, 150 mM NaCl and 0.05% Tween-20). To ensure that the observed immunoreactivity was specific to RGS18, peptide inhibition studies were performed. For the peptide inhibition studies, antisera was incubated in 100 mM Tris, pH 7.5 plus 1×EDTA-free Complete™ mini protease inhibitor cocktail (Roche Biochemicals, catalog # 183-6170) in the absence or presence of 100 mg/ml of the immunizing peptide or an unrelated peptide for 2 hours or overnight at 4° C. This was then diluted to the working concentration (1:500 or 1:1000) in blocking buffer (5% non-fat dry milk in TBST) and incubated with the nitrocellulose strips for 1 hour at room temperature (RT) and developed as described above. Whole cell lysates for western blotting were made from platelets, leukocytes and the three megakaryocyte cell lines by lysing cells in 50 mM HEPES, pH 8.0, 6 MM MgCl₂, 300 mM NaCl, 1 mM DTT, 1% Triton X-100 and 1×EDTA-free Complete™ mini protease inhibitor cocktail (Roche Biochemicals, catalog # 183-6170). Cells were lysed on ice, spun for 10 minutes at 13,000×g at 4° C. to pellet insoluble material, and the supernatants were transferred to fresh tubes. Protein determinations were performed using Bradford assay using BSA to generate the standard curve. Fifty micrograms of each lysate was run on 15% reducing SDS-PAGE, transferred to 0.2 mM nitrocellulose and blotted as above. For detection of RGS10 in cell lysates, SDS-PAGE was carried out as above and the resulting nitrocellulose strips were blocked in 2% BSA/TBST and incubated with 1:500 dilution of anti-RGS10 antibody (Santa Cruz Biotechnology, Santa Cruz, Calif.) in blocking buffer at RT for 2 hours or overnight at 4° C. Bound antibody was detected with mouse anti-goat IgG-coupled to horseradish peroxidase (HRP; Pierce, Rockford, EL) and Supersignal West Pico ECL reagent (Pierce, Rockford, Ill.).

Results of Tissue Distribution of RGS18 Nucleic Acids and Polypeptides in Human Tissues.

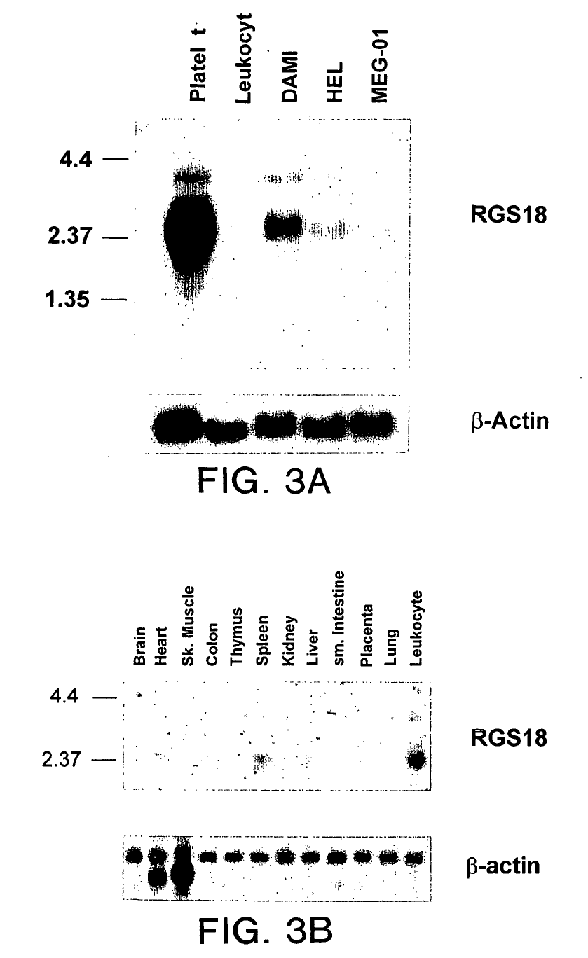

In order to determine the relative tissue distribution of RGS18, two Northern blots were hybridized with a 3′ untranslated region probe (SEQ ID NO: 6) from RGS18. The first blot contained 10 mg/lane of total RNA from human platelets, human leukocytes, DAMI cells, HEL cells and MEG-01 cells. The second blot was a commercially-available Human Multiple Tissue Northern (Clontech; Palo Alto, Calif.) containing 1 ug of poly(A)⁺ mRNA from 12 human tissues, one lane of which contained RNA from polymorphonuclear leukocytes. In order to ensure a fair comparison, the labeled probe was divided in half and used to probe both blots simultaneously. The RGS18 probe hybridizes to a major species at ˜2.75 Kb and minor species at ˜4.2 Kb in platelet RNA and to a lesser extent in DAMI, HEL, and MEG-01 cells (FIG. 3, panel A). Human leukocyte RNA also expresses both of these transcripts at levels equal to or slightly less than that in MEG-01 cells. Since complete separation of platelets and leukocytes is difficult, whenever expression levels of transcripts in platelet RNA are evaluated, one must be concerned with the level of contamination of the platelet RNA by the more abundant leukocyte RNA. The fact that leukocytes have such a low expression level of RGS18 compared to platelet RNA indicates that this is not a concern for this transcript. Therefore, platelets express significantly greater quantities of message for RGS18 in comparison to leukocytes, with the intermediate levels expressed in the megakaryocytic cell lines.

Results from the Human Multiple Tissue Northern Blot are shown in FIG. 3, panel B. Overall the hybridization signal was much lower than that seen with the platelet Northern blot above and required much longer exposures. For example, when both blots were hybridized with the same probe and same specific activity, the platelet Northern blot required only 6 hours of autoradiographic exposure versus the 6 days required for the Human Multiple Tissue Northern. On the Multiple Tissue blot, the most intense band was consistently in the leukocyte lane, followed by spleen, then heart and liver, and very low levels in skeletal muscle, colon, kidney, small intestine, placenta and lung. Hybridization of a Multiple tissue Northern containing RNA from other human tissues demonstrated moderate levels of expression of RGS18 in human bone marrow also, levels comparable to that seen in spleen (data not shown). These Northern blots were repeated at least twice on fresh blots to ensure that results were consistent. Using these data for comparison of the expression levels of RGS18, it appears that the level of expression of this message in platelets greatly exceeds that in leukocytes, which express RGS18 in excess of the other tissues examined. In support of this conclusion, we used leukocyte and bone marrow cDNA in the degenerate RT-PCR strategy for mining RGS transcripts, as was done for platelets, and none of the colonies sequenced contained RGS18. As would be predicted based on previous studies, the most abundant amplification products in leukocytes and bone marrow in these studies were hRGS2 and hRGS16 (data not shown).

Results of Western Blot Analysis of RGS18 Expression in Human Platelets.

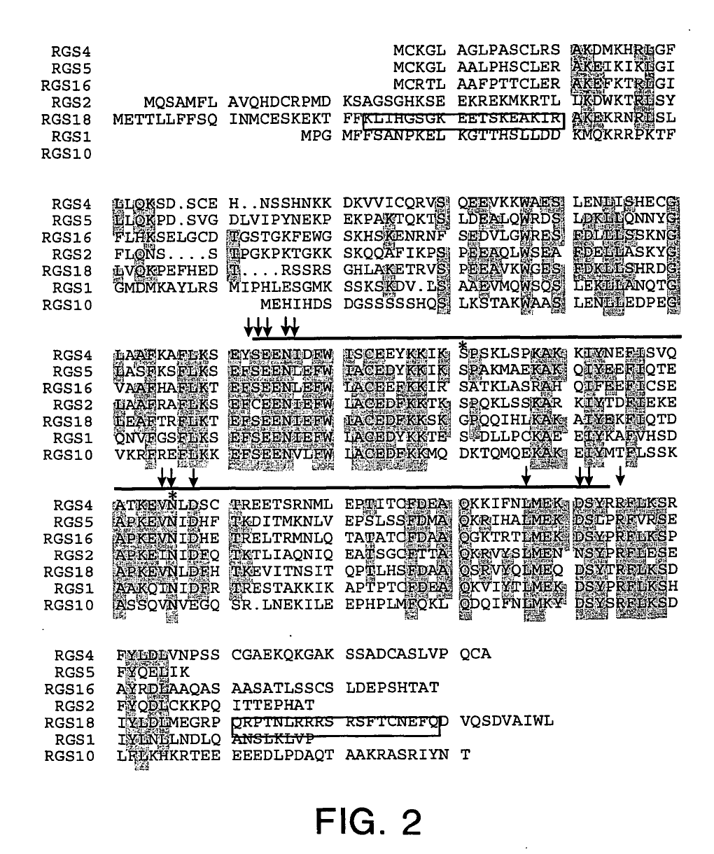

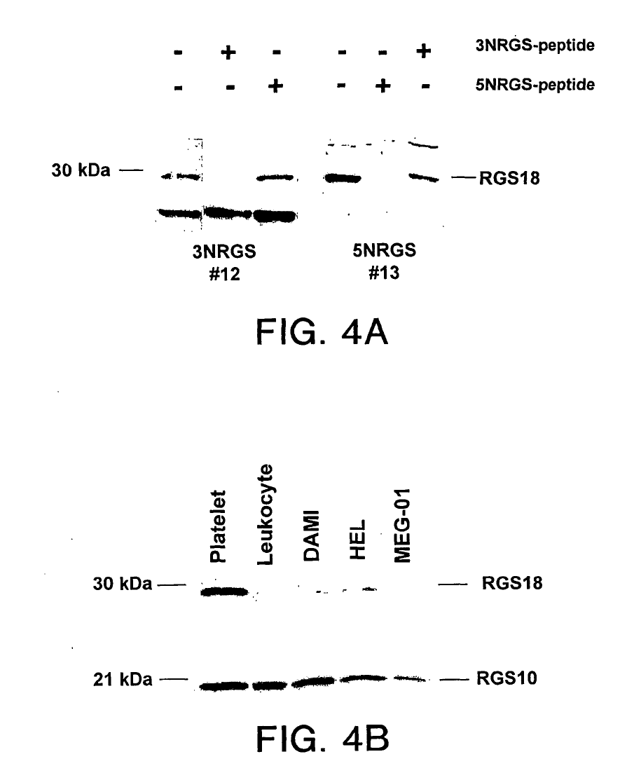

Polyclonal anti-RGS18 antisera were generated against two peptides, one in the amino-terminus (SEQ ID NO: 7) and the second in the carboxy-terminus (SEQ ID NO: 8) of RGS18. The location and sequences of these peptides is shown in the boxed regions of FIG. 2. These regions were selected due to their divergence from similar regions of the other known RGSs. The resulting sera were tested in western blots with platelet lysates for reactivity and specificity. Prior studies show that these polyclonal antibodies also react with recombinant RGS18 synthesized by coupled transcription/translation (data not shown). Antisera directed against both carboxy-terminal and amino-terminal peptides reacted with an ˜30 kDa band in platelet lysates. Preincubation of each antiserum with its corresponding immunizing peptide but not the other peptide ablates antibody reactivity with the 30 kDa protein, indicating that each antibody is indeed specific for RGS18 (FIG. 4, Panel A). Antisera 5NRGS-13 (directed against an amino-terminal peptide comprising SEQ ID NO: 7) has a higher titer than antisera 3NRGS-12 (directed against a carboxy-terminal peptide comprising SEQ ID NO: 8) and was used for further evaluation of protein expression levels. Western analysis was performed on lysates prepared from platelets, leukocytes and DAMI, HEL and MEG-01 cells to compare the relative protein expression levels of RGS18. FIG. 4, Panel B shows the results of a representative experiment using antisera against the amino terminal peptide, 5NRGS-13. Consistent with the northern expression data, RGS18 protein is significantly more abundant in platelets than in leukocytes, DAMI cells, HEL cells, or MEG-01 cells. Although an immunoreactive band in the MEG-1101 lane is not visible in FIG. 4, panel B, longer exposures do indeed demonstrate the expression of RGS18 in these cells. Northern expression data suggested that the levels of expression of RGS18 in MEG-01 cells may be similar to leukocytes, yet this was not observed in the western blot analysis. This may be due to the presence of contaminating platelets in the leukocytes preparation, which would lead to an overestimation of RGS18 expression in leukocytes. Indeed, western blots using an antibody against a platelet specific protein α_{2b} (GPIIb, CD41) demonstrate reactivity with leukocyte lysates, indicating the presence of some contaminating platelets in the leukocyte preparation (data not shown).

Since the presence of hRGS10 was detected by RT-PCR and an hRGS10 antibody was available, hRGS10 expression levels were examined by western blotting. Western blotting of platelet, leukocyte and megakaryocyte cell line lysates was performed as described above, using this anti-hRGS10 antibody, and indicated that hRGS10 is almost equivalently expressed in platelets, leukocytes and DAMI cells, with lower levels of expression in the other two megakaryocytic cell lines (FIG. 4, Panel B). hRGS10 has also been reported to be expressed in brain (163). Taken together, these data indicate that hRGS18 and hRGS10 are both expressed in platelets, and that hRGS18 but not hRGS10 is preferentially expressed in platelets versus leukocytes. Based on northern blotting data, the expression of hRGS18 in other human tissues would be predicted to be equal to or less than that seen in leukocytes, suggesting that hRGS18 likely has a restricted tissue distribution, with preferential expression in platelets.

### Example 4

### Expression and Purification of GST-Fusion Proteins

In order to create an in-frame GST fusion protein using the Bam HI and Xho I sites of the plasmid pGEX-5X-1, oligonucleotides were synthesized from the 5′ most and 3′ most coding regions with an in-frame Bam HI site on the 5′ primer and a Xho I site after the termination codon in the 3′ primer. These oligonucleotides, sense (5′-gttcggatccgagagaagatggaaacaacaftgcttttc-3′; SEQ ID NO: 9) and antisense (5′-gtgctcgagttaacataaacatgcgatatg-3′; SEQ ID NO: 10), were used in RT-PCR to amplify platelet RNA. The resultant PCR amplification product was fully sequenced for fidelity of the PCR reaction, subcloned into pGEX-5×-1 and transformed into competent BL-21 DE3) (Novagen, San Diego, Calif.). Expression and purification of the resultant fusion protein was carried out as described in the manufacturer's directions. Essentially, an overnight culture was diluted 1:100 in Luria Broth containing 100 mg/ml ampicillin (typically 500 ml to 1 liter) and grown, shaking at 30° C. until it reached an OD₆₀₀=0.6. The culture was then induced with 0.5 mM IPTG for 2 hours at 30° C. Cells were pelleted, washed one time in cold PBS and resuspended in PBS containing 100 mg/ml of lysozyme (ICN, Costa Mesa, Calif.), 50 mg/ml of DNAase (Gibco/BRL, Rockville, Md.) and 1×EDTA-free Complete™ mini protease inhibitor cocktail (Roche Biochemicals, catalog # 183-6170). The suspended cells were sonicated on ice and Triton X-100 was added to a final concentration of 1%. The cell lysate was mixed for 30 minutes at 4° C. followed by centrifugation at 12,000×g for 10 minutes at 4° C. The supernatant was transferred to a fresh tube and 2 ml of a 50% slurry of washed glutathione-Sepharose 4B was added and incubated at 4° C. for 1 hour. The matrix was then washed batch-wise 5-8 times in ice-cold PBS and resuspended to a 50% slurry in 50 mM HEPES, pH 7.4, 150 mM NaCl, 5 mM DTT, 10% glycerol and protease inhibitors and aliquot and stored frozen at −70° C. Prior to use the matrix was thawed, washed 2-3 times in PBS or lysis buffer and resuspended to a 50% slurry. When necessary, elution of GST-RGS18 was carried out by loading the Sepharose 4 B bound fusion protein into a column and eluting with 20 mM reduced glutathione in 100 mM Tris pH 8.0, 120 mM NaCl.

### Example 5

### G Protein Alpha Subunit Binding Assays

Determination of G protein alpha subunit binding specificity of RGS18 was carried out as described in Beadling et al. (164), with minor modifications. Washed platelets from Day 2 concentrate were lysed in 50 mM HEPES, pH 8.0, 300 mM NaCl, 1 mM DTT, 6 MM MgCl2, 1% Triton X-100, and 1× EDTA-free Complete™ mini protease inhibitor cocktail (Roche Biochemicals, catalog # 183-6170). Protein determination was done using Bradford Assay (BioRad, Hercules, Calif.) using BSA as a standard and lysates were adjusted to 1 mg/ml in lysis buffer prior to use. Cell lysates (450 ml) were activated with 30 mM GDP or 30 mM GDP plus 30 mM AlCl₃ and 100 mM NaF for 30 minutes at 30° C. Following the incubation, lysates were quickly spun in a microfuge to pellet actin which became insoluble upon activation of the platelet lysates. Lysates were transferred to fresh tubes and incubated with 20 ml of a 50% slurry of GST-RGS18 coupled to glutathione-sepharose 4B beads (typically ˜10 mg RGS18 protein) for 1 hour at 4° C. The beads were washed twice in 1 ml of wash buffer (50 mM HEPES, pH 8.0, 300 mM NaCl, 1 mM DTT, 6 mM MgCl₂, 0.025% C₁₂E₁₀, and 1× EDTA-free Complete™ mini protease inhibitor cocktail (Roche Biochemicals, catalog # 183-6170). Bound protein was eluted in two rounds of boiling in reducing Laemmli buffer (50 mM Tris-HCL pH 6.8, 1% SDS, 0.008% bromophenol blue, 5% glycerol) and subjected to SDS-PAGE on 12% gels, transferred to 0.45 mM nitrocellulose and blotted with antisera against G_{αi1/2} (Calbiochem, San Diego, Calif.), G_{αi3/O} (Calbiochem, San Diego, Calif.), G. (Santa Cruz Biotechnology, Santa Cruz, Calif.), G_{αz} (Santa Cruz Biotechnology, Santa Cruz, Calif.), G_{αq/11} (Santa Cruz Biotechnology, Santa Cruz, Calif.) or G_{αs} (Calbiochem, San Diego, Calif. and Santa Cruz Biotechnology, Santa Cruz, Calif.). Bound antibody was detected using goat anti-rabbit-BRP (BioRad, Hercules, Calif.) and Supersignal West Pico ECL reagent (Pierce, Rockford, Ill.).

Results of Binding Analysis of RGS18 to Endogenous G Protein Alpha Subunits in Human Platelets.

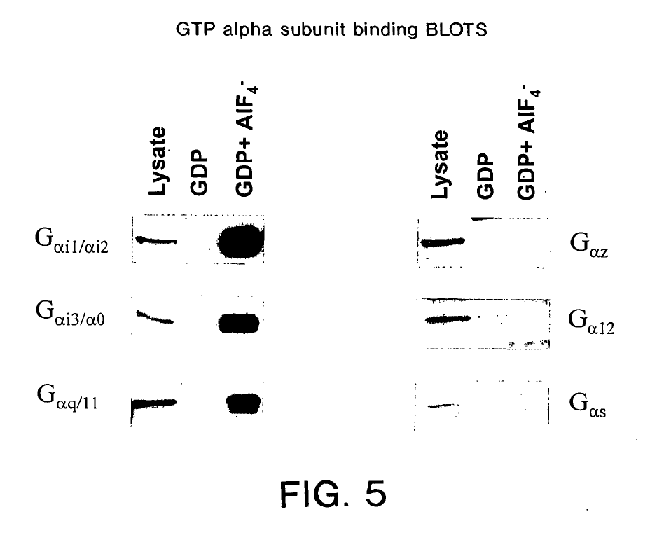

In order to determine the target alpha subunits of RGS18, binding experiments were performed using GST-RGS18 as prepared in Example 4 and lysates of human platelets. A similar method has been used to determine the binding specificity of hRGS16 using lysates from Jurkat cells (164). Previous work has demonstrated that RGS proteins studied to date bind G protein alpha subunits only when the alpha subunit is in its transition state or “activated state” (165). For these experiments, platelets were lysed in a Triton X-100 containing buffer and resuspended to 1 mg/ml. The lysate was then treated with GDP, which holds the alpha subunit in the GDP-bound “in-activated” state, or GDP+AlF₄⁻ that mimics the transition state of the G_{α} subunit. Recombinant GST-RGS18 bound to Sepharose-4 beads was added to the treated lysates, incubated and washed several times. Bound protein was eluted in Laemmli buffer and subjected to SDS-PAGE followed by western blot analysis. A panel of G protein alpha subunit specific antibodies was used to determine which alpha subunits bind to RGS18. FIG. 5 shows the results of a representative experiment. RGS18 binds little if any alpha subunit in the inactive GDP-bound state (middle lane of each panel). In contrast, in lysates that have been treated with GDP+AlF₄⁻, RGS18 binds a significant amount of alpha subunit detected by antibodies directed against G_{αi1/2}, G_{α0/i3} and G_{αq/11}. This interaction appears to be specific since RGS18 did not bind G_{αz}, G_{α12}, or G_{αs}. Neither GST nor Sepharose 4B alone binds to any of these alpha subunits in either GDP or GDP+AlF₄⁻ treated platelet lysates (data not shown). The binding selectivity of RGS18 is consistent with that found for other RGS proteins which selectively bind to members of the G_{αi} family and/or G_{αq} family (14).

### Example 6

### Homology Comparison of RGS18 with Other RGS Proteins

The predicted amino acid sequence of RGS18 (SEQ ID NO: 20) was compared to that of several other of the more closely related RGS proteins. FIG. 2 depicts an alignment of human RGS18 protein (SEQ ID NO: 20) with human (h) RGS4 (SEQ ID NO: 21), hRGS5 (SEQ ID NO: 22), hRGS16 (SEQ ID NO: 23), hRGS2 (SEQ ID NO: 24), hRGS1 (SEQ ID NO: 25), and hRGS10 (SEQ ID NO: 26), which was generated using the Pileup program from GCG. Shaded areas represent amino acids that are conserved between RGS18 and at least two other RGSs. The region of the RGS18 domain that was initially isolated by RT-PCR i.e., amino acids 1-77 and 79-81 of SEQ ID NO: 12, which correspond to amino acids 109-185 and 187-189 of SEQ ID NO: 20 is shown by a line above the amino acids in FIG. 2. Human RGS18 is most homologous to human RGS5 (47% identity), followed by rat RGS8 (SEQ ID NO: 27; 44%), hRGS2 (41%), hRGS4 and hRGS16 (40%), hRGS1 (37%/o), HRGS10 and hRGS3 (SEQ ID NO: 28; 36%) and hRGS13 (SEQ ID NO: 29; 34%). Human RGS18 displays between 20 to 30% identity to other known RGS proteins. Recent phylogenetic analysis of the RGS superfamily indicates the presence of at least six distinct subfamilies (166). RGS18 is most closely related to members of Family B. RGS proteins in Family B contain characteristic short amino and carboxy terminal domains (except RGS3) and two conserved amino acids (locations depicted by asterisks above residues in FIG. 2). The first is an asparagine (ASN) residue at position 128 in hRGS4 (SEQ ID NO: 21) found in families B, C and D and is conserved in hRGS18 (amino acid 152 of SEQ ID NO: 20). Family B is the most divergent of the group and contains only one subfamily-specific residue. A serine (Ser) is conserved between the known RGS family members [Ser 103 in hRGS4 (SEQ ID NO: 21)] however, this residue is not conserved in hRGS18 (glycine at amino acid 127 of SEQ ID NO: 20). All but one the residues in hRGS4 that have been shown to contact the G, subunit (18) are conserved between hRGS18 and hRGS4 (depicted by ↑ in FIG. 2).

### Discussion

Classically, the G protein signal transduction cascade consists of the integral membrane receptor, the heterotrimeric GTP-binding protein and the downstream effector. More recently, the complexity of these signaling pathways has become apparent by the identification of additional signaling molecules that regulate distinct aspects of these pathways. Several families of molecules have been identified which serve to attenuate receptor-mediated signaling at the receptor and/or G protein level (167). Regulators of G protein Signaling (RGS) are a new family of proteins which were identified in genetic studies of yeast and worms (nemaotodes) as negative regulators of G protein signaling, and many mammalian homologues have since been characterized (14). RGSs appear to regulate signaling via interaction with one or more G_{α} subunits, stabilizing the transition state of the G_{α} subunits, thereby accelerating GTP hydrolysis (19). In an effort to better understand GPCR-mediated signaling in human platelets, studies to examine which isoforms of the RGS family exist in platelets were conducted. Applicants describe herein the identification of nucleic acids encoding a novel Regulator of G protein Signaling, RGS18 which is abundantly expressed in human platelets.

Although platelets are enucleate cells, they contain small amounts of residual cytoplasmic RNA presumably carried over from their precursor cell, the megakaryocyte. Several groups have taken advantage of the presence of this residual RNA to do molecular biological identification of platelet proteins (168). Degenerate RT-PCR analysis is well suited to study transcript expression in platelets, due to the minimal requirement of RNA of this technique. Using degenerate primers (SEQ ID NOs: 1-4) that were designed based upon conserved amino acids of the amino and carboxy-terminal regions of the RGS domain, RGS transcripts expressed in platelet RNA were identified. Surprisingly, the majority of amplification products encoded a novel partial RGS domain (amino acids 1-81 of SEQ ID NO: 12 corresponding to amino acids 109-189 of SEQ ID NO: 20, with the exception of amino acid 78 of SEQ ID NO: 12). Platelets also appear to contain transcripts for other previously known RGS proteins including hRGS2, hRGS16 and hRGS10. Additionally, RNA from three megakaryocytic cell lines, Dami, HEL, and MEG-01 cells were analyzed using the same method, and in contrast to what was found in platelet RNA, the most abundant amplification product in each of these cells was hRGS16. One colony out of 50 analyzed in DAMI cells contained a PCR product comprising the novel partial RGS domain, indicating that these cells also express the novel RGS. These cells likely differ from platelets in their expression of RGSs due to the fact that they are undifferentiated, and leukemic in nature. This is not unexpected since several studies have demonstrated modulation of RGS expression in several different cell types. The expression levels of hRGS1 and hRGS2 can both be regulated by mitogens in lymphocytes (11, 12), and hRGS16 can regulated by IL-1 in lymphocytes (164) and the tumor suppressor, p53 in a colon carcinoma cell line (161). Although these cell lines have many features of the megakaryocytic lineage (i.e., expression of GPIIb/IIIa and other platelet specific genes) (169-171) and have proved useful for identification of platelet-specific genes, they are not perfect models of platelet signal transduction. Most importantly these cells do not appear to have intact signaling pathways which stimulate binding of soluble fibrinogen to GPIIb/IIIa, indicating that their signal transduction pathways or complement of signaling proteins differs from platelets (172). The most abundant RGS isoform in human bone marrow and peripheral blood leukocyte RNA detected by degenerate RT-PCR was hRGS2 (data not shown) that further points out the distinction between platelets and other hematopoetic cells. The fact that each tissue displays a different transcript profile is a good indication that the primers are not biased for one RGS isoform over others, and that the proportional expression we see with this method likely reflects relative RNA expression levels.

Although RGS18 was not detected in human peripheral blood leukocyte RNA by this degenerate RT-PCR method, it may be that this transcript was not in fact amplified from platelet RNA but from contaminating white blood cell RNA. Since platelets contain so little RNA, a small amount of white blood cell contamination could lead to a disproportionate contamination of the platelet RNA by leukocyte RNA. Northern blot analysis of human platelet and leukocyte RNA indicates that this is not the case, since platelet RNA expresses significantly higher levels of RGS18 than leukocytes. The megakaryocytic cell lines express levels of RGS18 intermediate to those of platelets and leukocytes. Examination of the expression of RGS18 by northern blotting in a wide variety of human tissues indicates that RGS18 appears to be most abundantly expressed in platelets, followed by leukocytes and then other tissues of the hematopoetic system, namely spleen and bone marrow, as well as heart and liver. Very low level expression can be detected in other tissues as well, including skeletal muscle, colon, kidney, small intestine, placenta and lung, but whether these levels translate to significant expression of the protein in these tissues remains to be determined. Two transcripts for RGS18, a major species of 2.75 Kb and a minor species of 4.2 Kb, are expressed in platelet RNA, as well as all other tissues examined. The presence of two mRNA species on the northern blot indicates that this transcript, like many others, is subject to alternative splicing and/or differential polyadenylation.

Full-length cloning of RGS18 was achieved by combining the sequence information from the initial PCR product (SEQ ID NO: 11) of the RGS domain, an overlapping Incyte EST cDNA (SEQ ID NO: 6) comprising the 3′ untranslated region and 5′ RACE cDNA (SEQ ID NO: 18) to obtain the entire coding region and some 5′ untranslated region of RGS18 (SEQ ID NO: 19). The open reading frame of the RGS18 cDNA (SEQ ID NO: 19) encodes a 235 amino acid protein (SEQ ID NO: 20) with a putative RGS domain (amino acids 86-202 of SEQ ID NO: 20). RGS18 has very short carboxy- and amino-terminal domains flanking the internal RGS domain and does not appear to contain functional domains for scaffolding (i.e., PH, Db1, GGL or DEP). It does however have one putative CAAX motif that might serve as a site of acylation, and permit membrane anchoring. RGS18 also contains several consensus sites for phosphorylation by the enzymes cAMP/cGMP dependent protein kinase, protein kinase C and casein kinase II. This indicates the potential for regulation of RGS18 by other signaling cascades.

Recently a phylogenetic analysis of this family has been performed and demonstrates that the RGS superfamily can be divided into at least 6 subfamilies (A through F) (166). RGS18 would most probably be a member of subfamily B, since it is most closely related to these RGSs, and like RGS18, subfamily B members characteristically contain short amino and carboxy-terminal domains. RGS18 does contain a highly conserved asparagine residue at position 152 of SEQ ID NO: 20 (relative position 128 in hRGS4; SEQ ID NO: 21) that is conserved in three of the six families. Structural studies of RGS4 indicate that this residue is critical for GAP activity and stabilization of the transition state of G_{α} (18, 173). Unlike some of the other subfamilies, subfamily B is a diverse group, and only one amino acid, a Ser residue (position 103 in hRGS4; SEQ ID NO: 21), is conserved between all the members of Family B. However, the corresponding residue in RGS18 (position 127 of SEQ ID NO: 20) is a glycine, which calls into question whether RGS18 is in fact a member of subfamily B. Interestingly, hRGS10 cannot be placed into one of the subfamilies due to its divergence from the other known RGSs. Recently, the sequence of a novel RGS that has been termed RGS17, isolated from a chicken dorsal root ganglion cDNA library was reported and is distinct from the platelet hRGS18 (31% amino acid identity), and in fact appears to be a member of subfamily A (174). Whether RGS18 belongs to Family B is as yet uncertain, and waits further functional and structural characterization. Seven of eight human RGSs of Family B appear to be clustered on chromosome 1, perhaps due to gene duplication events (166). It will be interesting to determine if RGS18 is also localized on this chromosome. As additional members of the RGS superfamily are identified, and as more information is gained about the functionality of each RGS, the defining characteristics of the various RGS subfamilies will become more distinct.

Since RNA expression levels do not always reflect protein expression levels, we thought it important to look at the expression of RGS18 in western blots using specific antisera. Two peptide-directed antisera were generated against peptides, one located in the amino terminus and a second in the carboxy-terminus of RGS18. RGS18 migrates on SDS-PAGE with an apparent molecular weight of 30 kDa and is abundantly expressed in platelets and to a significantly lesser extent in leukocytes and the three megakaryocytic cell lines. This reactivity can be neutralized by pre-incubating the antisera with the immunizing peptide, demonstrating that the reactivity of this 30 kDa band is specific for RGS18. The presence of RGS18 in commercially prepared lysates (Clontech, Palo Alto, Calif.) was not detected from human brain, liver or lung (data not shown). If RGS18 is expressed at all in these tissues, its level of expression is probably too low to detect with the currently available tools.

Platelets are known to express a variety of G_{α} subunits. Previous work has shown that platelets contain members of the G_{αi} family, G_{αi1}, G_{αi2}, G_{αi3}, G_{αz}, G_{α12/13}, G_{α16}, G_{αs} (5) and G_{αq} but not G_{α11} (175, 176). Since a source of recombinant G protein alpha subunits was unavailable, the G protein alpha subunit selectivity of RGS18 was analyzed using endogenous platelet G proteins. This strategy was used by Beadling et al., using Jurkat cell lysates and recombinant hRGS16, to determine the binding specificity of hRGS16 (164). A GST-tagged fusion protein of RGS18 binds G_{α} subunits detected by antibodies to G_{αi1/2}, G_{αi3/o} and G_{αq/11} in platelet lysates that are activated by treatment with GDP+AlF₄⁻. In these same experiments, RGS18 failed to interact with G_{α2}, G_{α12} or G_{αs}. Although the antisera have somewhat overlapping specificity for the G_{αi} subunits, platelets contain immunologically undetectable levels of G_{αi1} (177), so that the majority of G_{α} subunit detected by the first antisera is likely G_{αi2}. G_{αo} has not been reported in platelets, and therefore this antibody is most likely detecting interaction with G_{αi3}. G_{α11} is not expressed in platelets (175, 176), so the G_{αq/11} antibody is detecting the presence of G_{αq}. Taken together, these data indicate that RGS18 interacts with G_{αq}, G_{αi2} and/or G_{αi3} and likely regulates one or more pathways mediated by these G_{α} subunits in platelets.

This G_{α} subunit specificity is in line with what has been demonstrated for other RGSs which typically interact with members of the G_{αi} and/or G_{αq} family. No RGS has been identified which interacts with G_{αs}. The RGS protein, p115 RhoGEF, from the subfamily F appear to be the only members identified which interact with G_{α12/13} subunits (178, 179). Despite the fact that G_{αz} is abundantly expressed in platelets (180), RGS18 does not appear to interact with this alpha subunit. Interestingly, hRGS10 is expressed in platelets at both the RNA and protein levels. hRGS10 has been reported to interact with G_{αz} as well as G_{αi3} (181). It is likely that due to their somewhat distinct G_{α} preference, that RGS10 and RGS18 might serve to regulate different signaling pathways in platelets.

The exact function of RGS18 in platelet signal transduction is not clear. In vitro G_{α} subunit binding specificity indicates that RGS18 likely regulates pathways mediated via activation of both G_{αi}- and G_{αq}-linked pathways. In platelets, aggregation appears to be dependent upon concomitant activation of both G_{αi}- and G_{αq}-coupled receptors (37). The platelet agonists, thrombin, thromboxane A₂ and ADP are all linked to signaling pathways via these G_{α} subunits. Blockade of signaling through G_{αq} in mice, leads to a phenotype in which platelets are not responsive to a variety of agonists and as a result are susceptible to hemorrhage (39). Due to the ubiquitous expression of G_{αq}, these mice also display other deleterious phenotypes, including ataxia, making G_{αq} a poor target for anti-platelet therapy. Because of its potential to regulate the G protein-mediated pathways in platelets that are critical for platelet activation, and the fact that it is enriched in platelets over tissues, RGS18 or an as yet unknown protein which regulates RGS18, might make a good target for therapies aimed at regulating platelet activation. Future studies addressing the regulation of individual GPCRs and their signal transduction pathways by RGS18, and other RGSs which are present in platelets, will give us a better understanding of the role that RGSs play in platelet activation events.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

### REFERENCES CITED

1. Bourne (1997) Curr. Opin. Cell Biol., 9:134-142.

2. Wess (1997) FASEB. J., 11: 346-354.

3. Bockaert and Pin (1999) Embo J., 18:1723-1729.

4. Hamm (1998) J. Biol. Chem., 273: 669-672.

5. Brass, L. F., Manning, D. R., Cichowski, K., and Abrams, C. S. (1997) Thromb. Haemost. 78, 581-9

6. Dohlman H. G., Song J., Ma D., Courchesne W. E., Thorner J. (1996) Mol. Cell. Biol. 16, 5194-209

7. Yu J. H., Wieser J., Adams T. H. (1996) EMBO. J. 15:5184-90

8. Koelle M. R. and Horvitz H. R.(1996) Cell 84:115-25

9. De Vries L, Mousli M., Wurmser A., Farquhar M. G. (1995) Proc. Natl. Acad. Sc.i U.S.A. 92: 11916-20

10. Druey K. M., Blumer K. J., Kang V. H., Kehrl J. H. (1996) Nature 379: 742-6

11. Newton J. S., Deed R. W., Mitchell E. L., Murphy J. J., Norton J. D. (1993 Biochim. Biophys. Acta. 1216: 314-6.

12. Wu H. K., Heng H. H., Shi X. M., Forsdyke D. R., Tsui L. C., Mak T. W., Minden M. D., Siderovski D. P. (1995) Leukemia 9: 1291-8

13. Hong J. X., Wilson G. L., Fox C. H., Kehrl J. H. (1993) J. Immunol. 150: 3895-904

14. Hepler J. R. (1999) Pharmacol. Sci. 20: 376-82

15. Gilman A. G. (1987) Annu. Rev. Biochem. 56:615-49

16. Berman D. M., Wilkie T. M., Gilman A. G. (1996) Cell 86: 445-52

17. Watson N., Linder M. E., Druey K. M., Kehrl J. H., Blumer K. J. (1996) Nature 383:172-5

18. Tesmer J J, Berman D M, Gilman A G, Sprang S R (1997) Cell 89:251-61

19 Berman D. M., Kozasa T., Gilman A. G. (1996) J. Biol. Chem. 271: 27209-12.

20. Hepler J. R., Berman D. M., Gilman A. G., Kozasa T. (1997 Proc. Natl. Acad. Sci. U.S.A. 94: 428-32)

21. Yan Y., Chi P. P., Bourne H. R. (1997) J. Biol. Chem. 272:11924-7

22. Huang C., Hepler J. R., Gilman A. G., Mumby S. M. (1997) Proc. Natl. Acad. Sci. U.S.A. 94: 6159-63

23. Neill J. D., Duck L. W., Sellers J. C., Musgrove L. C., Scheschonka A, Druey K. M., Kehrl J. H. (1997) Endocrinology 138: 843-6

24. Saugstad J. A., Marino M. J., Folk J. A., Hepler J. R., Conn P. J. (1998) J. Neurosci. 18: 905-13

25. Makino E. R., Handy J. W., Li T., Arshavsky V. Y. (1999) Proc. Natl. Acad. Sci. U.S. A. 96:1947-52

26. Nekrasova E. R., Berman D. M., Rustandi R. R., Hamm H. E., Gilman A. G., Arshavsky V. Y. (1997) Biochemistry 36: 7638-43

27. Daniel J. L., Dangelmaier C., Jin J., Ashby B., Smith J. B., Kunapuli S. P. (1998) Molecular basis for ADP-induced platelet activation. J. Biol. Chem. 273: 2024-9

28. Jin J., Daniel J. L., Kunapuli S. P. (1998 J. Biol. Chem. 273: 2030-4

29. Benka M. L., Lee M., Wang G. R., Buckman S., Burlacu A., Cole L., DePina A., Dias P., Granger A., Grant B., et al (1995) FEBS Lett. 363: 49-52

30. Brass L. F., Laposata M., Banga H. S., Rittenhouse S. E. (1986) J. Biol. Chem. 261: 16838-47

31. Offermanns S., Laugwitz K. L., Spicher K., Schultz G. (1994) Proc. Natl. Acad. Sci. U.S. A. 91: 504-8

32. Kinsella B. T., O'Mahony D. J., Fitzgerald G. A. (1997) J. Pharmacol. Exp. Ther. 281: 957-64

33. Ushikubi F., Nakamura K., Narumiya S. (1994) Functional reconstitution of platelet thromboxane A2 receptors with Gq and Gi2 in phospholipid vesicles. Mol. Pharmacol. 46: 808-16

34. Baldassare J. J., Tarver A. P., Henderson P. A., Mackin W. M., Sahagan B., Fisher G. J. (1993) Biochem. J. 291: 235-40

35. Knezevic I., Borg C., Le Breton G. C. (1993) J. Biol. Chem. 268: 26011-7

36. Djellas Y., Manganello J. M., Antonakis K., Le Breton G. C. (1999). J. Biol. Chem. 274: 14325-30

37. Jin J., Kunapuli S. P. (1998) Proc. Natl. Acad. Sci. U.S. A. 95: 8070-4

38. Jantzen H. M., Gousset L., Bhaskar V., Vincent D., Tai A., Reynolds E. E., Conley P. B. (1999) Thromb. Haemost. 81: 111-7

39. Offermanns S, Toombs C F, Hu Y H, Simon M I (1997) Nature 389:183-6.

40. Sambrook, J. Fritsch, E. F., and T. Maniatis, 1989. Molecular cloning: a laboratory manual. 2ed. Cold Spring Harbor Laboratory, Cold spring Harbor, N.Y.

41. Glover (ed.), 1985. DNA Cloning: A Practical Approach, Volumes I and II Oligonucleotide Synthesis, MRL Press, Ltd., Oxford, U.K.

42. Gait (ed.), 1984. Nucleic Acid Hybridization.

43. Hames B D and Higgins S J, 1985. Nucleic acid hybridization: a practical approach, Hames and Higgins Ed., IRL Press, Oxford.

44. Hames and Higgins (eds.), 1984. Animal Cell Culture.

45. Freshney (ed.), 1986. Immobilized Cells And Enzymes, IRL Press.

46. Perbal, 1984. A Practical Guide To Molecular Cloning.

47. Ausubel et al., 1989. Current Protocols in Molecular Biology, Green Publishing Associates and Wiley Interscience, N.Y.

48. Reeck et al., 1987, Cell 50:667.

49. Maniatis et al., 1982. Molecular cloning: A Laboratory Manual, Cold Spring Harbor Press, Cold Spring, N.Y.

50. Altschul et al, 1990. J. Mol. Biol., 215:403-410.

51. Altschul et al, 1997. Nucleic Acids Res., 25:3389-3402.

52. Benton and Davis, 1977, Science 196:180.

53. Grunstein and Hogness, 1975, Proc. Natl. Acad. Sci. U.S.A. 72:3961.

54. Hutchinson, C., et al., 1978, J. Biol. Chem. 253:6551.

55. Zoller and Smith, 1984, DNA 3:479-488.

56. Oliphant et al., 1986, Gene 44:177.

57. Hutchinson et al., 1986, Proc. Natl. Acad. Sci. U.S.A. 83:710.

58. Huygen et al., 1996. Nature Medicine, 2(8):893-898.

59. Higuchi, 1989, “Using PCR to Engineer DNA”, in PCR Technology: Principles and Applications for DNA Amplification, H. Erlich, ed., Stockton Press, Chapter 6, pp. 61-70.

60. Benoist and Chambon, 1981, Nature 290:304-310.

61. Yamamoto, et al., 1980, Cell 22:787-797.

62. Wagner et al., 1981, Proc. Natl. Acad. Sci. U.S.A. 78:1441-1445.

63. Brinster et al., 1982, Nature 296:39-42.

64. Villa-Kamaroff, et al., 1978, Proc. Natl. Acad. Sci. U.S.A. 75:3727-3731.

65. DeBoer, et al., 1983, Proc. Natl. Acad. Sci. U.S.A. 80:21-25.

66. Swift et al., 1984, Cell 38:639-646.

67. Ornitz et al., 1986, Cold Spring Harbor Symp. Quant. Biol. 50:399-409.

68. MacDonald, 1987, Hepatology 7:425-515.

69. Hanahan, 1985, Nature 315:115-122.

70. Grosschedl et al., 1984, Cell 38:647-658.

71. Adames et al., 1985, Nature 318:533-538.

72. Alexander et al., 1987, Mol. Cell. Biol. 7:1436-1444.

73. Leder et al., 1986, Cell 45:485-495.

74. Pinkert et al., 1987, Genes and Devel. 1:268-276.

75. Krumlauf et al., 1985, Mol. Cell. Biol. 5:1639-1648.

76. Hammer et al., 1987, Science 235:53-58.

77. Kelsey et al., 1987, Genes and Devel. 1:161-171.

78. Mogram et al., 1985, Nature 315:338-340.

79. Kollias et al., 1986, Cell 46:89-94.

80. Readhead et al., 1987, Cell 48:703-712.

81. Sani, 1985, Nature 314:283-286.

82. Mason et al., 1986, Science 234:1372-1378.

83. Smith et al., 1988, Gene 67:31-40.

84. Kaufman, 1991. Current Protocols in Molecular Biology, 16.12.

85. Wu et al., 1992, J. Biol. Chem. 267:963-967.

86. Wu and Wu, 1988, J. Biol. Chem. 263:14621-14624.

87. Hartmut et al., Canadian Patent Application No. 2,012,311, filed Mar. 15, 1990.

88. Felgner et al., 1987. PNAS 84:7413.

89. Mackey, et al., 1988. Proc. Natl. Acad. Sci. U.S.A. 85:8027-8031.

90. Ulmer et al., 1993. Science 259:1745-1748.

91. Felgner and Ringold, 1989. Science 337:387-388.

92. Williams et al., 1991. Proc. Natl. Acad. Sci. USA 88:2726-2730.

93. Curiel et al., 1992. Hum. Gene Ther. 3:147-154.

94. Wu and Wu, 1987. J. Biol. Chem. 262:4429-4432.

95. Narang S A, Hsiung H M, Brousseau R, 1979. Methods Enzymol, 68:90-98.

96. Brown E L, Belagaje R, Ryan M J, Khorana H G, 1979. Methods Enzymol, 68:109-151.

97. Beaucage et al., 1981. Tetrahedron Lett, 22: 1859-1862.

98. Urdea M. S., 1988. Nucleic Acids Research, 11:49374957.

99. Sanchez-Pescador R., 1988. J. Clin. Microbiol., 26(10):1934-1938.

100. Urdea M S et al., 1991. Nucleic Acids Symp Ser., 24:197-200.

101. Fuller S. A. et al., 1996. Immunology, In: Current Protocols in Molecular Biology, Ausubel et al. (eds.).

102. Sternberg N. L., 1992. Trends Genet., 8:1-16.

103. Sternberg N. L., 1994. Mamm. Genome, 5:397404.

104. Flotte et al., 1992. Am. J. Respir. Cell Mol. Biol., 7:349-356.

105. Samulski et al., 1989. J. Virol., 63:3822-3828.

106. McLaughlin B A et al., 1996. Am. J. Hum. Genet., 59:561-569.

107. Graham et al., 1973. Virology, 52:456457.

108. Chen et al., 1987. Mol. Cell. Biol., 7: 2745-2752.

109. Gopal, 1985. Mol. Cell. Biol., 5:1188-1190.

110. Tur-Kaspa et al, 1986. Mol. Cell. Biol., 6:716-718.

111. Potter et al., 1984. Proc Natl Acad Sci USA., 81(22):7161-5.

112. Harland et al., 1985. J. Cell. Biol., 101:1094-1095.

113. Nicolau C. et al., 1987. Methods Enzymol., 149:157-76.

114. Fraley et al., 1979. Proc. Natl. Acad. Sci. USA, 76:3348-3352.

115. Tacson et al., 1996. Nature Medicine, 2(8):888-892.

116. Pagano et al., 1967. J. Virol., 1:891.

117. Kaneda et al., 1989. Science 243:375.

118. Fraley et al., 1980. J. Biol. Chem., 255:10431.

119. Beard et al., 1990. Virology, 75:81.

120. Levrero et al., 1991. Gene 101.

121. Graham, 1984. EMBO J., 3:2917.

122. Graham et al., 1977. J. Gen. Virol., 36:59.

123. Breakfield et al., 1991. New Biologist, 3:203.

124. Bernstein et al., 1985. Genet. Eng., 7:235.

125. McCormick, 1985. BioTechnology, 3:689.

126. Bender et al., 1987. J. Virol., 61:1639.

127. Houben Weyl, 1974. In: Meuthode der Organischen Chemie, E. Wunsch Ed., 15-I:15-II.

128. Merrifield R B, 1965a. Nature, 207(996):522-523.

129. Merrifield R B., 1965b. Science, 150(693):178-185.

130. Koch Y., 1977. Biochem. Biophys. Res. Commun., 74:488-491.

131. Kozbor et al., 1983. Hybridoma, 2(1):7-16.

132. Kohler G. and Milstein C., 1975. Nature, 256:495-497.

133. Kozbor et al., 1983. Immunology Today, 4:72.

134. Cote et al., 1983. Proc. Natl. Acad. Sci. U.S.A. 80:2026-2030.

135. Cole et al., 1985. In: Monoclonal Antibodies and Cancer Therapy, Alan R. Liss, Inc., pp. 77-96.

136. Morrison et al., EP 173494.

137. Neuberger et al., 1984. Nature, 312:604-608.

138. Takeda et al., 1985. Nature 314:452-454.

139. Huse et al., 1989. Science 246:1275-1281.

140. Martineau P, Jones P, Winter G, 1998. J Mol Biol, 280(1):117-127.

141. Ridder R, Schmitz R, Legay F, Gram H, 1995. Biotechnology (NY), 13(3):255-260.

142. Reimann K A, et al., 1997. AIDS Res Hum Retroviruses, 13(11):933-943.

143. Leger O J, et al., 1997. Hum Antibodies, 8(1):3-16.

144. Langer, 1990. Science, 249:1527-1533.

145. Treat et al., 1989. In: Liposomes in the Therapy of Infectious Disease and Cancer, Lopez-Berestein and Fidler (eds.), Liss: New York pp. 353-365.

146. Lopez-Berestein, 1989. In: Liposomes in the Therapy of Infectious Disease and Cancer, Lopez-Berestein and Fidler (eds.), Liss: New York, pp. 317-327.

147. Sefton, 1987. CRC Crit. Ref. Biomed. Eng. 14:201.

148. Buchwald et al., 1980. Surgery 88:507.

149. Saudek et al., 1989 N. Engl. J. Med. 321:574.

150. Langer and Wise (eds.), 1974. In: Medical Applications of Controlled Release, CRC Press: Boca Raton, Fla.

151. Smolen and Ball (eds.), 1984. Controlled Drug Bioavailability, Drug Product Design and Performance, Wiley: New York.

152. Ranger and Peppas, 1983. J. Macromol. Sci. Rev. Macromol. Chem. 23:61.

153. Levy et al., 1985. Science 228:190.

154. During et al., 1989. Ann. Neurol. 25:351.

155. Howard et al., 1989. J. Neurosurg. 71:105.

156. Goodson, 1984. In: Controlled Drug Bioavailability, Drug Product Design and Performance, Smolen and Ball (eds.), Wiley: New York, vol. 2, pp. 115-138.

157. Berman, et al., 1996. Cell 86:445-452.

158. Denecke et al., 1999. J. Biol. Chem. 274:26860-26868.

159. Huang et al., 1997. Proc. Natl. Acad. Sci. 94:6159-6163.

160. Chen C., Zheng B., Han J., Lin S. C. (1997) J. Biol. Chem. 272: 8679-85.

161. Buckbinder L, Velasco-Miguel S., Chen Y., Xu N., Talbott R., Gelbert L., Gao J., Seizinger B. R., Gutkind J. S., Kley N. (1997) Proc Natl. Acad. Sci. U.S.A. 94: 7868-72.

162. Snow B. E., Antonio L., Suggs S., Siderovski D. P. (1998) Gene 206: 247-53.

163. Gold S. J., Ni Y. G., Dohlman H. G., Nestler E. J. (1997) J. Neurosci. 17: 8024-37.

164. Beadling, C., Druey, K. M., Richter, G., Kerh; J. H., and Smith, K. A. (1999) J. Immunol. 162, 2677-2682.

165. Dohlman H. G., Thorner J. (1997) RGS proteins and signaling by heterotrimeric G proteins. J. Biol. Chem. 272: 3871-4.

166. Zheng B., De Vries L., Farquhar M. G. (1999) Trends. Biochem. Sci. 24: 411-4.

167. Bunemann M, Lee K B, Pals-Rylaarsdam R, Roseberry A G, Hosey M M (1999) Annu. Rev. Physiol. 61:169-92.

168. Newman P. J., Gorski J., White G. C. 2d, Gidwitz S., Cretney C. J., Aster R. H. (1988) J. Clin Invest. 82: 739-43.

169. Tabilio A., Rosa J. P., Testa U., Kieffer N., Nurden A. T., Del Canizo M. C., Breton-Gorius J, Vainchenker W. (1984) EMBO J. 3: 453-9.

170. Ogura M., Morishima Y., Ohno R., Kato Y, Hirabayashi N., Nagura H., Saito H. (1995) Blood 66: 1384-92.

171. Greenberg S. M., Rosenthal D. S., Greeley T. A., Tantravahi R., Handin R. I. (1988) Blood 72: 1968-77.

172. Boudignon-Proudhon C., Patel P. M., Parise L. V. (1996) Blood 87: 968-76.

173. Srinivasa S. P., Watson N., Overton M. C., Blumer, K. J. (1998) J. Biol. Chem. 273: 1529-1533.

174. Jordan J. D., Carey K. D., Stork P. J., Iyengar R. (1999) J. Biol. Chem 274: 21507-21510.

175. Milligan G., Mullaney I., McCallum J. F. (1993) Biochim. Biophys. Acta.; 1179: 208-212.

176. Johnson G. J., Leis L. A., Dunlop P. C. (1996) Biochem. J. 318:1023-31.

177. Williams A. G., Woolkalis M. J., Poncz M., Manning D. R., Gewirtz A. M., Brass L. F. (1990) Blood 76: 721-30.

178. Kozasa T., Jiang X., Hart M. J., Sternweis P. M., Singer W. D., Gilman A. G., Bollag G., Sternweis P. C. (1998) Science 280: 2109-11.

179. Hart M. J., Jiang X., Kozasa T., Roscoe W., Singer W. D., Gilman A. G., Sternweis P. C., Bollag G. (1998) Science 280: 21124.

180. Gagnon A. W., Manning D. R., Catani L., Gewirtz A., Poncz M., Brass L. F. (1991) Blood 78: 1247-53.

181. Hunt T. W., Fields T. A., Casey P. J., Peralta (1996) Nature, 383: 175-7.

1. An isolated nucleic acid comprising a polynucleotide sequence of a) any one of SEQ ID NOs: 11, 18, or 19, or of a complementary polynucleotide sequence, b) nucleotides 1-169 of SEQ ID NO: 11, or of a complementary polynucleotide sequence, c) nucleotides 1-658 of SEQ ID NO: 19, or of a complementary polynucleotide sequence, d) nucleotides 163-870 of SEQ ID NO: 19, or of a complementary polynucleotide sequence, e) nucleotides 163-658 of SEQ ID NO: 19, or of a complementary polynucleotide sequence, f) nucleotides 418-768 of SEQ ID NO: 19, or of a complementary polynucleotide sequence, or g) nucleotides 418-658 of SEQ ID NO: 19, or of a complementary polynucleotide sequence.

2. An isolated nucleic acid comprising at least eight consecutive nucleotides of a polynucleotide sequence of a) nucleotides 1-169 of SEQ ID NO: 11, or of a complementary polynucleotide sequence, b) nucleotides 1-658 of SEQ ID NO: 19, or of a complementary polynucleotide sequence, c) nucleotides 163-658 of SEQ ID NO: 19, or of a complementary polynucleotide sequence, or d) nucleotides 418-658 of SEQ ID NO: 19, or of a complementary polynucleotide sequence.

3. An isolated nucleic acid comprising at least 80% nucleotide identity with a nucleic acid comprising a) any one of SEQ ID NOs: 11, 18, or 19, or a complementary polynucleotide sequence, b) nucleotides 1-169 of SEQ ID NO: 11, or a complementary polynucleotide sequence, c) nucleotides 1-658 of SEQ ID NO: 19, or a complementary polynucleotide sequence, d) nucleotides 163-870 of SEQ ID NO: 19, or a complementary polynucleotide sequence, e) nucleotides 163-658 of SEQ ID NO: 19, or a complementary polynucleotide sequence, f) nucleotides 418-768 of SEQ ID NO: 19, or a complementary polynucleotide sequence, or g) nucleotides 418-658 of SEQ ID NO: 19, or a complementary polynucleotide sequence.

4. The isolated nucleic acid according to claim 3, wherein the nucleic acid comprises an 85%, 90%, 95%, or 98% nucleotide identity with the nucleic acid comprising a) any one of SEQ ID NOs: 11, 18, or 19, or a complementary polynucleotide sequence, b) nucleotides 1-169 of SEQ ID NO: 11, or a complementary polynucleotide sequence, c) nucleotides 1-658 of SEQ ID NO: 19, or a complementary polynucleotide sequence, d) nucleotides 163-870 of SEQ ID NO: 19, or a complementary polynucleotide sequence, e) nucleotides 163-658 of SEQ ID NO: 19, or a complementary polynucleotide sequence, f) nucleotides 418-768 of SEQ ID NO: 19, or a complementary polynucleotide sequence, or g) nucleotides 418-658 of SEQ ID NO: 19, or a complementary polynucleotide sequence.

5. An isolated nucleic acid that hybridizes under high stringency conditions with a nucleic acid comprising a) any one of SEQ ID NOs: 11, 18, or 19, or a complementary polynucleotide sequence, b) nucleotides 1-169 of SEQ ID NO: 11, or a complementary polynucleotide sequence, c) nucleotides 1-658 of SEQ ID NO: 19, or a complementary polynucleotide sequence, d) nucleotides 163-870 of SEQ ID NO: 19, or a complementary polynucleotide sequence, e) nucleotides 163-658 of SEQ ID NO: 19, or a complementary polynucleotide sequence, f) nucleotides 418-768 of SEQ ID NO: 19, or a complementary polynucleotide sequence, or g) nucleotides 418-658 of SEQ ID NO: 19, or a complementary polynucleotide sequence.

6. An isolated nucleic acid comprising a polynucleotide sequence as depicted in either one of SEQ ID NOs: 18 or 19, or of a complementary polynucleotide sequence.

7. A nucleotide probe or primer specific for an RGS18 nucleic acid, wherein the nucleotide probe or primer comprises at least 15 consecutive nucleotides of a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence.

8. The nucleotide probe or primer according to claim 7, wherein the nucleotide probe or primer comprises a marker compound.

9. A nucleotide probe or primer specific for an RGS18 nucleic acid, wherein the nucleotide probe or primer comprises a) any one of SEQ ID NOs: 9, 10, 14, 15, 16, 17, 30, 31, 32, 33, 34, 35, or 36, or of a complementary polynucleotide sequence, b) 1-169 of SEQ ID NO: 11, or of a complementary polynucleotide sequence, c) 1-658 of SEQ ID NO: 19, or of a complementary polynucleotide sequence, d) 163-658 of SEQ ID NO: 19, or of a complementary polynucleotide sequence, or e) 418-658 of SEQ ID NO: 19, or of a complementary polynucleotide sequence.

10. The nucleotide probe or primer according to claim 9, wherein the nucleotide probe or primer comprises a marker compound.

11. A method of amplifying a region of the nucleic acid according to claim 1, wherein the method comprises:
a) contacting the nucleic acid with two nucleotide primers, wherein the first nucleotide primer hybridizes at a position 5′ of the region of the nucleic acid, and the second nucleotide primer hybridizes at a position 3′ of the region of the nucleic acid, in the presence of reagents necessary for an amplification reaction; and
b) detecting the amplified nucleic acid region.

12. The method according to claim 11, wherein the two nucleotide primers are selected from the group consisting of
A) a nucleotide primer comprising at least 15 consecutive nucleotides of a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence, and
B) a nucleotide primer comprising a polynucleotide sequence of a) any one of SEQ ID NOs: 9, 10, 14, 15, 16, 17, 30, 31, 32, 33, 34, 35, or 36, or of a complementary polynucleotide sequence, b) 1-169 of SEQ ID NO: 11, or of a complementary polynucleotide sequence, c) 1-658 of SEQ ID NO: 19, or of a complementary polynucleotide sequence, d) 163-658 of SEQ ID NO: 19, or of a complementary polynucleotide sequence, or e) 418-658 of SEQ ID NO: 19, or of a complementary polynucleotide sequence.

13. A kit for amplifying the nucleic acid according to claim 1, wherein the kit comprises:
a) two nucleotide primers whose hybridization position is located respectively 5′ and 3′ of the region of the nucleic acid; and optionally,
b) reagents necessary for an amplification reaction.

14. The kit according to claim 13, wherein the two nucleotide primers are selected from the group consisting of
A) a nucleotide primer comprising at least 15 consecutive nucleotides of a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence, and
B) a nucleotide primer comprising a polynucleotide sequence of a) any one of SEQ ID NOs: 9, 10, 14, 15, 16, 17, 30, 31, 32, 33, 34, 35, or 36, or of a complementary polynucleotide sequence, b) 1-169 of SEQ ID NO: 11, or of a complementary polynucleotide sequence, c) 1-658 of SEQ ID NO: 19, or of a complementary polynucleotide sequence, d) 163-658 of SEQ ID NO: 19, or of a complementary polynucleotide sequence, or e) 418-658 of SEQ ID NO: 19, or of a complementary polynucleotide sequence.

15. A method of detecting a nucleic acid according to claim 1, wherein the method comprises:
A) contacting the nucleic acid with a nucleotide probe selected from the group consisting of
1) a nucleotide probe comprising at least 15 consecutive nucleotides of a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence, and
2) a nucleotide probe comprising a polynucleotide sequence of a) any one of SEQ ID NOs: 9, 10, 14, 15, 16, 17, 30, 31, 32, 33, 34, 35, or 36, or of a complementary polynucleotide sequence, b) 1-169 of SEQ ID NO: 11, or of a complementary polynucleotide sequence, c) 1-658 of SEQ ID NO: 19, or of a complementary polynucleotide sequence, d) 163-658 of SEQ ID NO: 19, or of a complementary polynucleotide sequence, or e) 418-658 of SEQ ID NO: 19, or of a complementary polynucleotide sequence, and
B) detecting a complex formed between the nucleic acid and the probe.

16. The method of detection according to claim 15, wherein the probe is immobilized on a support.

17. A kit for detecting the nucleic acid according to claim 1, wherein the kit comprises
A) a nucleotide probe selected from the group consisting of
1) a nucleotide probe comprising at least 15 consecutive nucleotides of a polynucleotide sequence of nucleotides a) 1-169 of SEQ ID NO: 11, or of a complementary polynucleotide sequence, b) 1-658 of SEQ ID NO: 19, or of a complementary polynucleotide sequence, c) 163-658 of SEQ ID NO: 19, or of a complementary polynucleotide sequence, or d) 418-658 of SEQ ID NO: 19, or of a complementary polynucleotide sequence, and
2) a nucleotide primer comprising a polynucleotide sequence of a) any one of SEQ ID NOs: 9, 10, 14, 15, 16, 17, 30, 31, 32, 33, 34, 35, or 36, or of a complementary polynucleotide sequence, b) 1-169 of SEQ ID NO: 11, or of a complementary polynucleotide sequence, c) 1-658 of SEQ ID NO: 19, or of a complementary polynucleotide sequence, d) 163-658 of SEQ ID NO: 19, or of a complementary polynucleotide-sequence, or e) 418-658 of SEQ ID NO: 19, or of a complementary polynucleotide sequence, and optionally,
B) a reagent necessary for a hybridization reaction.

18. The kit according to claim 17, wherein the probe is immobilized on a support.

19. A recombinant vector comprising the nucleic acid according to claim 1.

20. The recombinant vector according to claim 19, wherein the recombinant vector is an adenovirus.

21. A recombinant vector comprising the nucleic acid according to claim 6.

22. The recombinant vector according to claim 21, wherein the recombinant vector is an adenovirus.

23. A recombinant host cell comprising the nucleic acid according to claim 1.

24. A recombinant host cell comprising the nucleic acid according to claim 6.

25. A recombinant host cell comprising the recombinant vector according to claim 19.

26. A recombinant host cell comprising the recombinant vector according to claim 21.

27. An isolated nucleic acid encoding a polypeptide comprising an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

28. A recombinant vector comprising the nucleic acid according to claim 27.

29. A recombinant host cell comprising the recombinant vector according to claim 28.

30. A recombinant host cell comprising the nucleic acid according to claim 27.

31. An isolated polypeptide comprising an amino acid sequence of a) either one of SEQ ID NOs: 12 or 20, b) amino acids 1-58 of SEQ ID NO: 12, c) amino acids 1-166 of SEQ ID NO: 20, d) amino acids 86-202 of SEQ ID NO: 20, or e) amino acids 86-166 of SEQ ID NO: 20.

32. An antibody directed against the isolated polypeptide according to claim 31.

33. The antibody according to claim 32, wherein the antibody comprises a detectable compound.

34. An isolated polypeptide comprising an amino acid sequence as depicted in SEQ ID NO: 20.

35. An antibody directed against the isolated polypeptide according to claim 34.

36. The antibody according to claim 35, wherein the antibody comprises a detectable compound.

37. A method of detecting a polypeptide, wherein the method comprises
a) contacting the polypeptide with an antibody according to claim 32; and
b) detecting an antigen/antibody complex formed between the polypeptide and the antibody.

38. A diagnostic kit for detecting a polypeptide, wherein the kit comprises
a) the antibody according to claim 32; and
b) a reagent allowing detection of an antigen/antibody complex formed between the polypeptide and the antibody.

39. A pharmaceutical composition comprising the nucleic acid according to claim 1 and a physiologically compatible excipient.

40. A pharmaceutical composition comprising the nucleic acid according to claim 6 and a physiologically compatible excipient.

41. A pharmaceutical composition comprising the recombinant vector according to claim 19 and a physiologically compatible excipient.

42. A pharmaceutical composition comprising the recombinant vector according to claim 21 and a physiologically compatible excipient.

43. A pharmaceutical composition comprising the nucleic acid according to claim 27 and a physiologically compatible excipient.

44. A pharmaceutical composition comprising the recombinant vector according to claim 28 and a physiologically compatible excipient.

45. A pharmaceutical composition comprising the recombinant host cell according to claim 29 and a physiologically compatible excipient.

46. A pharmaceutical composition comprising the recombinant host cell according to claim 30 and a physiologically compatible excipient.

47. A pharmaceutical composition comprising the polypeptide according to claim 31 and a physiologically compatible excipient.

48. A pharmaceutical composition comprising the polypeptide according to claim 34 and a physiologically compatible excipient.

49. Use of the nucleic acid according to claim 1 for the manufacture of a medicament intended for the prevention or treatment of a platelet activation dysfunction.

50. Use of the nucleic acid according to claim 6 for the manufacture of a medicament for the prevention or treatment of a platelet activation dysfunction.

51. Use of the recombinant vector according to claim 19 for the manufacture of a medicament for the prevention or treatment of a platelet activation dysfunction.

52. Use of the recombinant vector according to claim 21 for the manufacture of a medicament intended for the prevention or treatment of a platelet activation dysfunction.

53. Use of the nucleic acid according to claim 27 for the manufacture of a medicament for the prevention or treatment of a platelet activation dysfunction.

54. Use of the recombinant vector according to claim 28 for the manufacture of a medicament for the prevention or treatment of a platelet activation dysfunction.

55. Use of the recombinant host cell according to claim 29 for the manufacture of a medicament for the prevention or treatment of a platelet activation dysfunction.

56. Use of the recombinant host cell according to claim 30 for the manufacture of a medicament for the prevention or treatment of a platelet activation dysfunction.

57. Use of the polypeptide according to claim 31 for the manufacture of a medicament intended for the prevention or treatment of a platelet activation dysfunction.

58. Use of the polypeptide according to claim 31 for screening an active ingredient for the prevention or treatment of a platelet activation dysfunction.

59. Use of a recombinant host cell expressing the polypeptide according to claim 31 for screening an active ingredient for the prevention or treatment of a platelet activation dysfunction.

60. An implant comprising the recombinant host cell according to claim 23.

61. An implant comprising the recombinant host cell according to claim 25.

62. An implant comprising the recombinant host cell according to claim 29.

63. A method of identifying a modulator, agonist, or antagonist of an RGS18 polypeptide in a sample comprising
a) incubating a labeled GTP-loaded G protein polypeptide with an RGS18 polypeptide with the sample;
b) measuring the rate or extent of GTP hydrolysis; and
c) comparing the rate or extent of GTP hydrolysis determined in step b) with a rate or extent of GTP hydrolysis measured with a reconstituted labeled GTP-loaded G protein polypeptide/RGS18 poylpeptide mixture that has not been previously incubated in the presence of the sample.

64. The method according to claim 63, wherein the labeled GTP-loaded G protein polypeptide of step a) is loaded with γ-³²P-GTP and the rate or extent of GTP hydrolysis of step b) is measured by determining the amount of free ³²Pᵢ released.

65. The method according to claim 63, wherein the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.

66. A method of identifying a modulator, agonist, or antagonist of an RGS18 polypeptide in a sample comprising
a) incubating a cell membrane fraction expressing an RGS18 polypeptide with a labeled GTP and the sample;
b) measuring the rate or extent of GTP hydrolysis; and
c) comparing the rate or extent of GTP hydrolysis determined in step b) with a rate or extent of, GTP hydrolysis measured with a cell membrane fraction expressing an RGS18 polypeptide that has not been previously incubated in the presence of the sample.

67. The method according to claim 66, wherein the cell membrane fraction is obtained from a cell that, either naturally or after transfecting the cell with an RGS18 encoding nucleic acid, expresses an RGS18 polypeptide, and isolating the cell's membrane.

68. The method according to claim 66, wherein the labeled GTP of step a) is labeled with γ-³²P and the rate or extent of GTP hydrolysis of step b) is measured by determining the amount of free ³²Pᵢ released.

69. The method according to claim 66, wherein the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.

70. A method of identifying a modulator, agonist, or antagonist of an RGS18 polypeptide in a sample comprising
a) incubating a cell expressing an RGS18 polypeptide with a labeled adenine and the sample;
b) measuring the amount of labeled cyclic AMP (cAMP) produced; and
c) comparing the amount of labeled cAMP measured in step b) with an amount of labeled cAMP measured with a cell expressing an RGS18 polypeptide that has not been previously incubated in the presence of the sample.

71. The method according to claim 70, wherein the cell expressing the RGS18 polypeptide is transfected with an RGS18 encoding nucleic acid.

72. The method according to claim 70, wherein the labeled adenine of step a) is ³H-adenine.

73. The method according to claim 70, wherein the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.

74. A method of identifying a modulator, agonist, or antagonist of an RGS18 polypeptide in a sample comprising
a) incubating a cell expressing an RGS18 polypeptide with a labeled inositol and the sample;
b) measuring the amount of labeled inositol triphosphate produced; and
c) comparing the amount of labeled inositol triphosphate measured in step b) with an amount of labeled inositol triphosphate measured with a cell expressing an RGS18 polypeptide that has not been previously incubated in the presence of the sample.

75. The method according to claim 74, wherein the cell expressing the RGS18 polypeptide is transfected with an RGS18 encoding nucleic acid.

76. The method according to claim 74, wherein the labeled inositol of step a) is ³H-inositol.

77. The method according to claim 74, wherein the RGS18 polypeptide comprises an amino acid sequence selected from the group consisting of SEQ ID NO: 12, SEQ ID NO: 20, amino acids 1-58 of SEQ ID NO: 12, amino acids 1-166 of SEQ ID NO: 20, amino acids 86-202 of SEQ ID NO: 20, and amino acids 86-166 of SEQ ID NO: 20.


SEQUENCE LISTING (38 sequences)

SEQ ID NO: 1 - DNA, 21 bp, Artificial
  Synthetic PCR primer
grngaraayhtngarttytgg

SEQ ID NO: 2 - DNA, 21 bp, Artificial
  Synthetic PCR primer
grngaraayhtnmgnttytgg

SEQ ID NO: 3 - DNA, 19 bp, Artificial
  Synthetic PCR primer
grtangartynttytycat

SEQ ID NO: 4 - DNA, 19 bp, Artificial
  Synthetic PCR primer
grtarctrtynttytycat

SEQ ID NO: 5 - DNA, 30 bp, Artificial
  Synthetic PCR primer
cgctagggccttagactccttgcttcttcc

SEQ ID NO: 6 - DNA, 1486 bp, Homo sapiens
ctcaacctaccctccacagttttgatgctgcacaaagcagagtgtatcagctcatggaacaagacagttatacacgttttctgaaatctgacatctatttagacttgatggaaggaagacctcagagaccaacaaatcttaggagacgatcacgctcatttacctgcaatgaattccaagatgtacaatcagatgttgccatttggttataaagaaaattgattttgctcatttttatgacaaacttatacatctgcttctaacatatcgcatgtttatgttaagatttggtcccatcctttaaactgaaatatgtcatgtgaaattattttaaaaatgtaaaaacaaaactttctgctaacaaaatacatacagtatctgccagtatattctgtaaaaccttctatttgatgtcattccatttataatcagaaaaaaaacttatttcttaatcaaaaggcagtacaaaaaaagtaataatgttttataagattgtagagttaagtaaaagttaagcttttgcaaagttgtcaaaagttcaaacaaaagtctagttgggattttttaccaaagcagcataatatgtgttatataaacataataatactcagatatccaaatgttcagatagcatttttcataatgaatgttctcttttttttggtaatagtgtagaagtgatctggttcttacaatgggagatgaagaacatttattattgggttactactaaccctgtcccaagaatagtaatatcacctctagttataagccagcaacaggaacttttgtgaagacacattcatctctacagaacttcagattaaatataatctagattaatgactgagaataagatccacatttgaactcattcctaagtgaacatggacgtacccagttatacaaagtacttctgttggtcacagaaacatgaccagattttgcatatctccaggtagggaactaagtagactaccttatcaccggctaagaaaacttgctactaaactattaggccatcaatgggctggaataaaaaccgagaagtttttcccaggacgtctcatgtttggccctttagaattggggtagaaatcagaaatgagatgaggggaagaagcaaggagtctaaggccctagcgatttgggcatctgccacattggttcatattcagaaagtgttatctcattgattatattcttgttaagcaaatctccttaagtaattattattcaaataagattatactcatacatctatatgtcactgttttaaagagatatttaatttttaatgtgtgttacatggtctgtaaatatttgtatttaaaaatgccatgcattaggctttggaaatttaatgttagttgaaatgtaaaatgtgaaaactttagatcatttgtagtaataaatatttttaacttcattcatacaattaagtttatctgacaataaaagctctgactgaaaaaaaaaaaaaaaa

SEQ ID NO: 7 - protein, 17 aa, Homo sapiens
KLIHGSGEETSKEAKIR

SEQ ID NO: 8 - protein, 19 aa, Homo sapiens
QRPTNLRRRSRSFTCNEFQ

SEQ ID NO: 9 - DNA, 39 bp, Artificial
  Synthetic PCR Primer
gttcggatccgagagaagatggaaacaacattgcttttc

SEQ ID NO: 10 - DNA, 30 bp, Artificial
  Synthetic PCR Primer
gtgctcgagttaacataaacatgcgatatg

SEQ ID NO: 11 - DNA, 241 bp, Homo sapiens
gaggaaaatctggagttctggatagcctgtgaagatttcaagaaaagcaagggacctcaacaaattcaccttaaagcaaaagcaatatatgagaaatttatacagactgatgccccaaaagaggttaaccttgattttcacacaaaagaagtcattacaaacagcatcactcaacctaccctccacagttttgatgctgcacaaagcagagtgtatcagctcatggaaaacgacagctatc

SEQ ID NO: 12 - protein, 81 aa, Homo sapiens
LEENLEFWIACEDFKKSKGPQQIHLKAKAIYEKFIQTDAPKEVNLDFHTKEVITNSITQPTLHSFDAAQSRVYQLMENDSY

SEQ ID NO: 13 - protein, 69 aa, Homo sapiens
QPTLHSFDAAQSRVYQLMEQDSYTRFLKSDIYLDLMEGRPQRPTNLRRRSRSFTCNEFQDVQSDVAIWL

SEQ ID NO: 14 - DNA, 21 bp, Artificial
  Synthetic PCR Primer
atagcctgtgaagatttcaag

SEQ ID NO: 15 - DNA, 21 bp, Artificial
  Synthetic PCR Primer
tggcaacatctgattgtacat

SEQ ID NO: 16 - DNA, 21 bp, Artificial
  Synthetic Primer
aagtttgtcataaaaatgagc

SEQ ID NO: 17 - DNA, 21 bp, Artificial
  Synthetic PCR Primer
ttaacataaacatgcgatatg

SEQ ID NO: 18 - DNA, 1840 bp, Homo sapiens
gcagagacagaaagaaacgcagctcttgactgcttttttgtaaacattactgtaagagttgtgataactttttattctactatgtatatgtatggaatagtattaataaatgaactagggaaggatgtaataaattagacatctcttcattttagagagaagatggaaacaacattgcttttcttttctcaaataaatatgtgtgaatcaaaagaaaaaacttttttcaagttaatacatggttcaggaaaagaagaaacaagcaaagaagccaaaatcagagctaaggaaaaaagaaatagactaagtcttcttgtgcagaaacctgagtttcatgaagacacccgctccagtagatctgggcacttggccaaagaaacaagagtctcccctgaagaggcagtgaaatggggtgaatcatttgacaaactgctttcccatagagatggactagaggcttttaccagatttcttaaaactgaattcagtgaagaaaatattgaattttggatagcctgtgaagatttcaagaaaagcaagggacctcaacaaattcaccttaaagcaaaagcaatatatgagaaatttatacagactgatgccccaaaagaggttaacctcgattttcacacaaaagaagtcattacaaacagcatcactcaacctaccctccacagttttgatgctgcacaaagcagagtgtatcagctcatggaacaagacagttatacacgttttctgaaatctgacatctatttagacttgatggaaggaagacctcagagaccaacaaatcttaggagacgatcacgctcatttacctgcaatgaattccaagatgtacaatcagatgttgccatttggttataaagaaaattgattttgctcatttttatgacaaacttatacatctgcttctaacatatcgcatgtttatgttaagatttggtcccatcctttaaactgaaatatgtcatgtgaaattattttaaaaatgtaaaaacaaaactttctgctaacaaaatacatacagtatctgccagtatattctgtaaaaccttctatttgatgtcattccatttataatcagaaaaaaaacttatttcttaatcaaaaggcagtacaaaaaaagtaataatgttttataagattgtagagttaagtaaaagttaagcttttgcaaagttgtcaaaagttcaaacaaaagtctagttgggattttttaccaaagcagcataatatgtgttatataaacataataatactcagatatccaaatgttcagatagcatttttcataatgaatgttctcttttttttggtaatagtgtagaagtgatctggttcttacaatgggagatgaagaacatttattattgggttactactaaccctgtcccaagaatagtaatatcacctctagttataagccagcaacaggaacttttgtgaagacacattcatctctacagaacttcagattaaatataatctagattaatgactgagaataagatccacatttgaactcattcctaagtgaacatggacgtacccagttatacaaagtacttctgttggtcacagaaacatgaccagattttgcatatctccaggtagggaactaagtagactaccttatcaccggctaagaaaacttgctactaaactattaggccatcaatgggctggaataaaaaccgagaagtttttcccaggacgtctcatgtttggccctttagaattggggtagaaatcagaaatgagatgaggggaagaagcaaggagtctaaggccctagcgatttgggcatctgccacattggttcatattcagaaag

SEQ ID NO: 19 - DNA, 2144 bp, Homo sapiens
gcagagacagaaagaaacgcagctcttgactgcttttttgtaaacattactgtaagagttgtgataactttttattctactatgtatatgtatggaatagtattaataaatgaactagggaaggatgtaataaattagacatctcttcattttagagagaagatggaaacaacattgcttttcttttctcaaataaatatgtgtgaatcaaaagaaaaaacttttttcaagttaatacatggttcaggaaaagaagaaacaagcaaagaagccaaaatcagagctaaggaaaaaagaaatagactaagtcttcttgtgcagaaacctgagtttcatgaagacacccgctccagtagatctgggcacttggccaaagaaacaagagtctcccctgaagaggcagtgaaatggggtgaatcatttgacaaactgctttcccatagagatggactagaggcttttaccagatttcttaaaactgaattcagtgaagaaaatattgaattttggatagcctgtgaagatttcaagaaaagcaagggacctcaacaaattcaccttaaagcaaaagcaatatatgagaaatttatacagactgatgccccaaaagaggttaacctcgattttcacacaaaagaagtcattacaaacagcatcactcaacctaccctccacagttttgatgctgcacaaagcagagtgtatcagctcatggaacaagacagttatacacgttttctgaaatctgacatctatttagacttgatggaaggaagacctcagagaccaacaaatcttaggagacgatcacgctcatttacctgcaatgaattccaagatgtacaatcagatgttgccatttggttataaagaaaattgattttgctcatttttatgacaaacttatacatctgcttctaacatatcgcatgtttatgttaagatttggtcccatcctttaaactgaaatatgtcatgtgaaattattttaaaaatgtaaaaacaaaactttctgctaacaaaatacatacagtatctgccagtatattctgtaaaaccttctatttgatgtcattccatttataatcagaaaaaaaacttatttcttaatcaaaaggcagtacaaaaaaagtaataatgttttataagattgtagagttaagtaaaagttaagcttttgcaaagttgtcaaaagttcaaacaaaagtctagttgggattttttaccaaagcagcataatatgtgttatataaacataataatactcagatatccaaatgttcagatagcatttttcataatgaatgttctcttttttttggtaatagtgtagaagtgatctggttcttacaatgggagatgaagaacatttattattgggttactactaaccctgtcccaagaatagtaatatcacctctagttataagccagcaacaggaacttttgtgaagacacattcatctctacagaacttcagattaaatataatctagattaatgactgagaataagatccacatttgaactcattcctaagtgaacatggacgtacccagttatacaaagtacttctgttggtcacagaaacatgaccagattttgcatatctccaggtagggaactaagtagactaccttatcaccggctaagaaaacttgctactaaactattaggccatcaatgggctggaataaaaaccgagaagtttttcccaggacgtctcatgtttggccctttagaattggggtagaaatcagaaatgagatgaggggaagaagcaaggagtctaaggccctagcgatttgggcatctgccacattggttcatattcagaaagtgttatctcattgattatattcttgttaagcaaatctccttaagtaattattattcaaataagattatactcatacatctatatgtcactgttttaaagagatatttaatttttaatgtgtgttacatggtctgtaaatatttgtatttaaaaatgccatgcattaggctttggaaatttaatgttagttgaaatgtaaaatgtgaaaactttagatcatttgtagtaataaatatttttaacttcattcatacaattaagtttatctgacaataaaagctctgactgaaaaaaaaaaaaaaaa

SEQ ID NO: 20 - protein, 235 aa, Homo sapiens
METTLLFFSQINMCESKEKTFFKLIHGSGKEETSKEAKIRAKEKRNRLSLLVQKPEFHEDTRSSRSGHLAKETRVSPEEAVKWGESFDKLLSHRDGLEAFTRFLKTEFSEENIEFWIACEDFKKSKGPQQIHLKAKAIYEKFIQTDAPKEVNLDFHTKEVITNSITQPTLHSFDAAQSRVYQLMEQDSYTRFLKSDIYLDLMEGRPQRPTNLRRRSRSFTCNEFQDVQSDVAIWL

SEQ ID NO: 21 - protein, 205 aa, Homo sapiens
MCKGLAGLPASCLRSAKDMKHRLGFLLQKSDSCEHNSSHNKKDKVVICQRVSQEEVKKWAESLENLISHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLVNPSSCGAEKQKGAKSSADCASLVPQCA

SEQ ID NO: 22 - protein, 181 aa, Homo sapiens
MCKGLAALPHSCLERAKEIKIKLGILLQKPDSVGDLVIPYNEKPEKPAKTQKTSLDEALQWRDSLDKLLQNNYGLASFKSFLKSEFSEENLEFWIACEDYKKIKSPAKMAEKAKQIYEEFIQTEAPKEVNIDHFTKDITMKNLVEPSLSSFDMAQKRIHALMEKDSLPRFVRSEFYQELIK

SEQ ID NO: 23 - protein, 204 aa, Homo sapiens
MCRTLAAFPTTCLERAKEFKTRLGIFLHKSELGCDTGSTGKFEWGSKHSKENRNFSEDVLGWRESFDLLLSSKNGVAAFHAFLKTEFSEENLEFWLACEEFKKIRSATKLASRAHQIFEEFICSEAPKEVNIDHETRELTRMNLQTATATCFDAAQGKTRTLMEKDSYPRFLKSPAYRDLAAQASAASATLSSCSLDEPSHTAT

SEQ ID NO: 24 - protein, 211 aa, Homo sapiens
MQSAMFLAVQHDCRPMDKSAGSGHKSEEKREKMKRTLLKDWKTRLSYFLQNSSTPGKPKTGKKSKQQAFIKPSPEEAQLWSEAFDELLASKYGLAAFRAFLKSEFCEENIEFWLACEDFKKTKSPQKLSSKARKIYTDFIEKEAPKEINIDFQTKTLIAQNIQEATSGCFTTAQKRVYSLMENNSYPRFLESEFYQDLCKKPQITTEPHAT

SEQ ID NO: 25 - protein, 199 aa, Homo sapiens
MPGMFFSANPKELKGTTHSLLDDKMQKRRPKTFGMDMKAYLRSMIPHLESGMKSSKSKDVLSAAEVMQWSQSLEKLLANQTGQNVFGSFLKSEFSEENIEFWLACEDYKKTESDLLPCKAEEIYKAFVHSDAAKQINIDFRTRESTAKKIKAPTPTCFDEAQKVIYTLMEKDSYPRFLKSHIYLNLLNDLQANSLKLVP

SEQ ID NO: 26 - protein, 167 aa, Homo sapiens
MEHIHDSDGSSSSSHQSLKSTAKWAASLENLLEDPEGVKRFREFLKKEFSEENVLFWLACEDFKKMQDKTQMQEKAKEIYMTFLSSKASSQVNVEGQSRLNEKILEEPHPLMFQKLQDQIFNLMKYDSYSRFLKSDLFLKHKRTEEEEEDLPDAQTAAKRASRIYNT

SEQ ID NO: 27 - protein, 180 aa, Rattus norvegicus
MAALLMPRRNKGMRTRLGCLSHKSDSCSDFTAILPDKPNRALKRLSTEEATRWADSFDVLLSHKYGVAAFRAFLKTEFSEENLEFWLACEEFKKTRSTAKLVTKAHRIFEEFVDVQAPREVNIDFQTREATRKNMQEPSLTCFDQAQGKVHSLMEKDSYPRFLRSKMYLDLLSQSQRRLS

SEQ ID NO: 28 - protein, 519 aa, Homo sapiens
MFETEADEKREMALEEGKGPGAEDSPPSKEPSPGQELPPGQDLPPNKDSPSGQEPAPSQEPLSSKDSATSEGSPPGPDAPPSKDVPPCQEPPPAQDLSPCQDLPAGQEPLPHQDPLLTKDLPAIQESPTRDLPPCQDLPPSQVSLPAKALTEDTMSSGDLLAATGDPPAAPRPAFVIPEVRLDSTYSQKAGAEQGCSGDEEDAEEAEEVEEGEEGEEDEDEDTSDDNYGERSEAKRSSMIETGQGAEGGLSLRVQNSLRRRTHSEGSLLQEPRGPCFASDTTLHCSDGEGAASTWGMPSPSTLKKELGRNGGSMHHLSLFFTGHRKMSGADTVGDDDEASRKRKSKNLAKDMKNKLGIFRRRNESPGAPPAGKADKMMKSFKPTSEEALKWGESLEKLLVHKYGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPPL

SEQ ID NO: 29 - protein, 159 aa, Homo sapiens
MSRRNCWICKMCRDESKRPPSNLTLEEVLQWAQSFENLMATKYGPVVYAAYLKMEHSDENIQFWMACETYKKIASRWSRISRAKKLYKIYIQPQSPREINIDSSTRETIIRNIQEPTETCFEEAQKIVYMHMERDSYPRFLKSEMYQKLLKTMQSNNSF

SEQ ID NO: 30 - DNA, 24 bp, Artificial
  Synthetic Sequencing Primer
ctactatgtatatgtatggaatag

SEQ ID NO: 31 - DNA, 22 bp, Artificial
  Synthetic Sequencing Primer
gaattttggatagcctgtgaag

SEQ ID NO: 32 - DNA, 24 bp, Artificial
  Synthetic Sequencing Primer
cgatcacgctcatttacctgcaat

SEQ ID NO: 33 - DNA, 24 bp, Artificial
  Synthetic Sequencing Primer
ctgaaatatgtcatgtgaaattat

SEQ ID NO: 34 - DNA, 21 bp, Artificial
  Synthetic Sequencing Primer
cataaacatgcgatatgttag

SEQ ID NO: 35 - DNA, 21 bp, Artificial
  Synthetic Sequencing Primer
tggggcatcagtctgtataaa

SEQ ID NO: 36 - DNA, 21 bp, Artificial
  Synthetic Sequencing Primer
cataaacatgcgatatgttag

SEQ ID NO: 37 - DNA, 21 bp, Artificial
  Synthetic Sequencing Primer
tggggcatcagtctgtataaa

SEQ ID NO: 38 - DNA, 21 bp, Artificial
  Synthetic Sequencing Primer
cctgaaccatgtattaacttg